SEQUENCE LISTING (53 sequences)

SEQ ID NO: 1 - DNA, 20 bp, synthetic construct
  source 1..20: mol_type = other DNA
ccctcgttttgccatgaacc

SEQ ID NO: 2 - DNA, 20 bp, synthetic construct
  source 1..20: mol_type = other DNA
tgccgaagcagttcttgctg

SEQ ID NO: 3 - DNA, 20 bp, synthetic construct
  source 1..20: mol_type = other DNA
agcagaatttttccaagcag

SEQ ID NO: 4 - DNA, 22 bp, synthetic construct
  source 1..22: mol_type = other DNA
ctcctttctctcttctttttcc

SEQ ID NO: 5 - DNA, 20 bp, synthetic construct
  source 1..20: mol_type = other DNA
gggatcccaggatctcacaa

SEQ ID NO: 6 - DNA, 20 bp, synthetic construct
  source 1..20: mol_type = other DNA
tgtgcagagcgataaccagg

SEQ ID NO: 7 - DNA, 20 bp, synthetic construct
  source 1..20: mol_type = other DNA
gcagcggagagtccttccag

SEQ ID NO: 8 - DNA, 20 bp, synthetic construct
  source 1..20: mol_type = other DNA
gggccaaacgtgtcttccag

SEQ ID NO: 9 - DNA, 21 bp, synthetic construct
  source 1..21: mol_type = other DNA
gagtgggtctgtgacgaggat

SEQ ID NO: 10 - DNA, 21 bp, synthetic construct
  source 1..21: mol_type = other DNA
ggttgtataggatgcgaggct

SEQ ID NO: 11 - DNA, 19 bp, synthetic construct
  source 1..19: mol_type = other DNA
gagggcgacttttggctac

SEQ ID NO: 12 - DNA, 20 bp, synthetic construct
  source 1..20: mol_type = other DNA
gtacccgtccagctcctctt

SEQ ID NO: 13 - DNA, 20 bp, synthetic construct
  source 1..20: mol_type = other DNA
ccttggatccaggagaacgg

SEQ ID NO: 14 - DNA, 20 bp, synthetic construct
  source 1..20: mol_type = other DNA
aagagtgagcccagcagaac

SEQ ID NO: 15 - DNA, 20 bp, synthetic construct
  source 1..20: mol_type = other DNA
gccttctttgagttcggtgg

SEQ ID NO: 16 - DNA, 20 bp, synthetic construct
  source 1..20: mol_type = other DNA
gaaatcaaacagaggccgca

SEQ ID NO: 17 - DNA, 22 bp, synthetic construct
  source 1..22: mol_type = other DNA
caatacggaatatcaatcccag

SEQ ID NO: 18 - DNA, 20 bp, synthetic construct
  source 1..20: mol_type = other DNA
gatcggaactattggttgtc

SEQ ID NO: 19 - DNA, 22 bp, synthetic construct
  source 1..22: mol_type = other DNA
cagaagggaaagtaaacatgag

SEQ ID NO: 20 - DNA, 20 bp, synthetic construct
  source 1..20: mol_type = other DNA
atcattcattccaacatgcc

SEQ ID NO: 21 - DNA, 21 bp, synthetic construct
  source 1..21: mol_type = other DNA
cattaactatgggaccttcac

SEQ ID NO: 22 - DNA, 20 bp, synthetic construct
  source 1..20: mol_type = other DNA
tccaggttctgaatgacttc

SEQ ID NO: 23 - DNA, 20 bp, synthetic construct
  source 1..20: mol_type = other DNA
taaggacgaaacgggactgg

SEQ ID NO: 24 - DNA, 21 bp, synthetic construct
  source 1..21: mol_type = other DNA
agtttggtggctggagcttta

SEQ ID NO: 25 - DNA, 19 bp, synthetic construct
  source 1..19: mol_type = other DNA
gggcctcttctgcgatttc

SEQ ID NO: 26 - DNA, 21 bp, synthetic construct
  source 1..21: mol_type = other DNA
atccaggcaagtgcattggta

SEQ ID NO: 27 - DNA, 20 bp, synthetic construct
  source 1..20: mol_type = other DNA
gaaccgcgagttcttttcaa

SEQ ID NO: 28 - DNA, 20 bp, synthetic construct
  source 1..20: mol_type = other DNA
aggacgcacttcacagatcc

SEQ ID NO: 29 - DNA, 20 bp, synthetic construct
  source 1..20: mol_type = other DNA
cgcttccttacctggttgat

SEQ ID NO: 30 - DNA, 20 bp, synthetic construct
  source 1..20: mol_type = other DNA
gagcgaccaaaggaaccata

SEQ ID NO: 31 - DNA, 80 bp, synthetic construct
  source 1..80: mol_type = other DNA
agtctgttctcctgtctcagtagtggaacgttgcatacctattccataaaagctgctccgtacggaggatcgacgttttg

SEQ ID NO: 32 - DNA, 80 bp, synthetic construct
  source 1..80: mol_type = other DNA
agtctgttctcctgtctcaggtactgtgacgcgttatttcgggcagaataaggctgtgcatacggaggatcgacgttttg

SEQ ID NO: 33 - DNA, 80 bp, synthetic construct
  source 1..80: mol_type = other DNA
agtctgttctcctgtctcagatctggctagagcgcatagagacgtgcaaagcactgtgaatacggaggatcgacgttttg

SEQ ID NO: 34 - DNA, 80 bp, synthetic construct
  source 1..80: mol_type = other DNA
agtctgttctcctgtctcagctgagacgcgcacgaatgatttacagcagtaagccgtgaatacggaggatcgacgttttg

SEQ ID NO: 35 - DNA, 80 bp, synthetic construct
  source 1..80: mol_type = other DNA
agtctgttctcctgtctcaggacgtggggctacgcctagcaacactgtaaacggtaaggttacggaggatcgacgttttg

SEQ ID NO: 36 - DNA, 80 bp, synthetic construct
  source 1..80: mol_type = other DNA
agtctgttctcctgtctcagattggaacgatccatctctagattcagccaggtccgcgcatacggaggatcgacgttttg

SEQ ID NO: 37 - DNA, 80 bp, synthetic construct
  source 1..80: mol_type = other DNA
agtctgttctcctgtctcagccgagacgcgcatttgcgctgtaaaataccacataccttttacggaggatcgacgttttg

SEQ ID NO: 38 - DNA, 80 bp, synthetic construct
  source 1..80: mol_type = other DNA
agtctgttctcctgtctcagatgagacgtccgtaggactgtaactcagagccctcgagtgtacggaggatcgacgttttg

SEQ ID NO: 39 - DNA, 80 bp, synthetic construct
  source 1..80: mol_type = other DNA
agtctgttctcctgtctcagaaactggaacgttctatttcacagcggctcgcctggtccatacggaggatcgacgttttg

SEQ ID NO: 40 - DNA, 80 bp, synthetic construct
  source 1..80: mol_type = other DNA
agtctgttctcctgtctcagtcagagatagagttcaagttcagtaccccgttttcgcaactacggaggatcgacgttttg

SEQ ID NO: 41 - DNA, 80 bp, synthetic construct
  source 1..80: mol_type = other DNA
agtctgttctcctgtctcagctgaaagggccgactggattatgcatctgtcgaatgtggttacggaggatcgacgttttg

SEQ ID NO: 42 - DNA, 80 bp, synthetic construct
  source 1..80: mol_type = other DNA
agtctgttctcctgtctcagcgccgtgtaaccctgcgaacatatttcctgatgagaagcctacggaggatcgacgttttg

SEQ ID NO: 43 - DNA, 80 bp, synthetic construct
  source 1..80: mol_type = other DNA
agtctgttctcctgtctcagattacgaacgcactctgtcgtccatcccttggaggaagtctacggaggatcgacgttttg

SEQ ID NO: 44 - DNA, 60 bp, synthetic construct
  source 1..60: mol_type = other DNA
tagtggaacgttgcatacctattccataaaagctgctccgtacggaggatcgacgttttg

SEQ ID NO: 45 - DNA, 67 bp, synthetic construct
  source 1..67: mol_type = other DNA
agtctgttctcctgtctcagtagtggaacgttgcatacctattccataaaagctgctccgtacggag

SEQ ID NO: 46 - DNA, 47 bp, synthetic construct
  source 1..47: mol_type = other DNA
tagtggaacgttgcatacctattccataaaagctgctccgtacggag

SEQ ID NO: 47 - DNA, 47 bp, synthetic construct
  source 1..47: mol_type = other DNA
cacacttctaagagggaacagacgtcgatctttgcggaactcctgcg

SEQ ID NO: 48 - DNA, 29 bp, synthetic construct
  source 1..29: mol_type = other DNA
gacccacggaggttgcgtccgtgggggac

SEQ ID NO: 49 - RNA, 21 nt, synthetic construct
  source 1..21: mol_type = other RNA
atagtcgcgactggtcgacta

SEQ ID NO: 50 - RNA, 19 nt, synthetic construct
  source 1..19: mol_type = other RNA
gcgactggttacccggtcg

SEQ ID NO: 51 - DNA, 47 bp, synthetic construct; identical to SEQ ID NO: 47
  source 1..47: mol_type = other DNA

SEQ ID NO: 52 - DNA, 29 bp, synthetic construct
  source 1..29: mol_type = other DNA
gacccacggaggttgcgtccgtgggggac

SEQ ID NO: 53 - DNA, 80 bp, synthetic construct
  source 1..80: mol_type = other DNA
agtctgttctcctgtctcagtagtggaacgttgcatacctcttccataaaagctgctccgtacggaggatcgacgttttg

(19) United States
(12) Patent Application Publication
(10) Pub. No.: US 2026/0306684 A1
(43) Pub. Date: Oct. 8, 2026

(54) METHODS AND MATERIALS FOR TREATING CANCER

(71) Applicant: Mayo Foundation for Medical Education and Research, Rochester, MN (US)

(72) Inventors: Rory L. Smoot, Rochester, MN (US); Tushar C. Patel, Ponte Vedra Beach, FL (US); Sumera I. Ilyas, Rochester, MN (US)

(21) Appl. No.: 19/108,341

(22) PCT Filed: Sep. 14, 2023
(86) PCT No.: PCT/US2023/032696

§ 371 Date: Mar. 3, 2025

Related U.S. Application Data
(60) Provisional application No. 63/406,527, filed on Sep. 14, 2022.

(51) Int. Cl. A61K 51/12; A61K 47/69; A61K 49/00; A61P 35/00

(52) U.S. Cl. CPC A61K 51/1224; A61K 47/6917; A61K 49/008; A61P 35/00

(57) ABSTRACT

This document relates to methods and materials for treating cancer. For example, this document provides vesicles (e.g., nanovesicles) designed to deliver one or more (e.g., one, two, three, or more) inhibitors to a cancer cell within a mammal (e.g., a human) having cancer to treat the mammal's cancer.

### CROSS-REFERENCE TO RELATED APPLICATIONS

This application claims the benefit of U.S. Patent Application Ser. No. 63/406,527, filed on Sep. 14, 2022. The disclosure of the prior application is considered part of, and is incorporated by reference in, the disclosure of this application.

### STATEMENT REGARDING FEDERAL FUNDING

This invention was made with government support under W81XWH-21-1-0798 awarded by the Department of Defense. The government has certain rights in the invention.

### SEQUENCE LISTING

This application contains a Sequence Listing that has been submitted electronically as an XML file named “07039-2160WO1.xml.” The XML file, created on Sep. 7, 2023, is 1256000 bytes in size. The material in the XML file is hereby incorporated by reference in its entirety.

### TECHNICAL FIELD

This document relates to methods and materials involved in treating cancer. For example, this document provides vesicles (e.g., nanovesicles) designed to deliver one or more (e.g., one, two, three, or more) inhibitors to a cancer cell within a mammal (e.g., a human) having cancer to treat the mammal.

### BACKGROUND INFORMATION

Although immunotherapy has transformed the care of cancer patients across a variety of cancers, it has not been an effective option for all cancers. For instance, immunotherapy has had limited success in poorly immunogenic cancers such as cholangiocarcinoma. Cholangiocarcinoma is a heterogeneous and aggressive malignancy, accounting for approximately 15% of all primary liver cancers with an overall 5-year survival rate of less than 10% (Banales et al., Nat. Rev. Gastroenterol. Hepatol., 17:557-588 (2020)). The treatment options for cholangiocarcinoma and other immune ‘cold tumors’ are limited. For example, surgical resection is possible in a minority of cholangiocarcinoma patients, however recurrence rates are nearly 70%, suggesting a high-rate of metastases even in those patients with clinically localized disease.

### SUMMARY

This document provides methods and materials for treating cancer. For example, this document provides vesicles (e.g., nanovesicles) designed to deliver one or more (e.g., one, two, three, or more) inhibitors to a cancer cell within a mammal (e.g., a human) having cancer to treat the mammal's cancer. In some cases, a vesicle provided herein can have (a) a targeting moiety located on a surface of the vesicle and (b) one or more (e.g., one, two, three, or more) inhibitors located within the vesicle. In some cases, a mammal (e.g., a human) having cancer (e.g., cholangiocarcinoma) can be administered one or more vesicles provided herein (e.g., one or more vesicles that include (a) a targeting moiety located on a surface of the vesicle(s) and (b) one or more (e.g., one, two, three, or more) inhibitors located within the vesicle) to treat the mammal's cancer.

As described herein, a population of one or more vesicles (e.g., nanovesicles) provided herein (e.g., one or more vesicles that include (a) a targeting moiety located on a surface of the vesicle(s) and (b) one or more (e.g., one, two, three, or more) inhibitors located within the vesicle) can be administered to a mammal (e.g., a human) having cancer (e.g., cholangiocarcinoma). For example, vesicles can be designed to include (a) a targeting moiety located on a surface of the vesicle and (b1) one or more inhibitors of a LCK proto-oncogene, an inhibitor of a Src family tyrosine kinase (LCK) polypeptide alone or in combination with one or more other inhibitors, (b2) one or more inhibitors of a TNF-related apoptosis-inducing ligand (TRAIL; also referred to as cluster of differentiation 253 (CD253) or tumor necrosis factor (ligand) superfamily, member 10 (TNFSF10)) polypeptide alone or in combination with one or more other inhibitors, or (b3) a combination of at least three inhibitors such that the combination inhibits at least three different polypeptides selected from the group consisting of (i) a LCK polypeptide, (ii) a TRAIL polypeptide, (iii) a Yes1 associated transcriptional regulator (YAP1) polypeptide, and (iv) a WW Domain Containing Transcription Regulator 1 (TAZ; encoded by WWTR1) polypeptide. Such designed vesicles can be administered to a mammal (e.g., a human) having cancer (e.g., cholangiocarcinoma) to treat the mammal's cancer. The vesicles (e.g., nanovesicles) provided herein (e.g., one or more vesicles that include (a) a targeting moiety located on a surface of the vesicle(s) and (b) one or more (e.g., one, two, three, or more) inhibitors located within the vesicle) can target and enter cells (e.g., cancer cells). For example, a targeting moiety located on a surface of a vesicle provided herein can bind to a molecule present on a surface of a cancer cell such that the vesicle enters the cancer cell via endocytic mechanisms (e.g., pinocytosis). When a vesicle provided herein (e.g., a vesicle having (a) a targeting moiety located on a surface of the vesicle and (b) one or more (e.g., one, two, three, or more) inhibitors located within the vesicle) enters a cell (e.g., a cancer cell), the one or more (e.g., one, two, three, or more) inhibitors located within the vesicle can inhibit the targeted one or more polypeptide(s) (e.g., can inhibit expression or activity of the one or more targeted polypeptide(s)) located within the cell. Having the ability to target cancer cells and inhibit particular polypeptides within the cancer cells (e.g., polypeptides involved in immune checkpoint pathways such as the TRAIL pathway and the PD-1/L1 pathway) provides a unique and unrealized opportunity to treat cancers (e.g., immunoresistant cancers).

In general, one aspect of this document features vesicles including a first inhibitor located within the vesicle, a second inhibitor located within the vesicle, a third inhibitor located within the vesicle, and a targeting moiety located on a surface of the vesicle, where the first inhibitor inhibits expression or activity of a first polypeptide, where the second inhibitor inhibits expression or activity of a second polypeptide, where the third inhibitor inhibits expression or activity of a third polypeptide, where the first polypeptide, the second polypeptide, and the third polypeptide are selected from the group consisting of a YAP polypeptide, a TAZ polypeptide, a LCK polypeptide, and a TRAIL polypeptide, where the targeting moiety can bind to a molecule present on the surface of a cancer cell, and where the vesicle has a longest diameter of 5 nm to 500 nm. In some embodiments, the first polypeptide can be a YAP polypeptide, the second polypeptide can be a TAZ polypeptide, and the third polypeptide can be LCK polypeptide. In some embodiments, the first polypeptide can be YAP polypeptide, the second polypeptide can be a TAZ polypeptide, and the third polypeptide can be a TRAIL polypeptide. In some embodiments, the first polypeptide can be a YAP polypeptide, the second polypeptide can be a LCK polypeptide, and the third polypeptide can be a TRAIL polypeptide. In some embodiments, the first polypeptide can be a TAZ polypeptide, the second polypeptide can be a LCK polypeptide, and the third polypeptide can be a TRAIL polypeptide. One of the first polypeptide, the second polypeptide, and the third polypeptide can be a YAP polypeptide, and one of the first inhibitor, the second inhibitor, and the third inhibitor can inhibit expression or activity of the YAP polypeptide. One of the first inhibitor, the second inhibitor, and the third inhibitor can be an siRNA molecule that can inhibit expression of the YAP polypeptide. One of the first inhibitor, the second inhibitor, and the third inhibitor can inhibit activity of the YAP polypeptide. One of the first inhibitor, the second inhibitor, and the third inhibitor can be CA3 or verteporfin. One of the first polypeptide, the second polypeptide, and the third polypeptide can be a TAZ polypeptide, and one of the first inhibitor, the second inhibitor, and third inhibitor can inhibit expression or activity of the TAZ polypeptide. One of the first inhibitor, the second inhibitor, and the third inhibitor can be an siRNA molecule that can inhibit expression of the TAZ polypeptide. One of the first inhibitor, the second inhibitor, and the third inhibitor can inhibit activity of the TAZ polypeptide. One of the first polypeptide, the second polypeptide, and the third polypeptide can be a LCK polypeptide, and one of the first inhibitor, the second inhibitor, and the third inhibitor can inhibit expression or activity of the LCK polypeptide. One of the first inhibitor, the second inhibitor, and the third inhibitor can be an siRNA molecule that can inhibit expression of the LCK polypeptide. One of the first inhibitor, the second inhibitor, and the third inhibitor can inhibit activity of the LCK polypeptide. One of the first inhibitor, the second inhibitor, and the third inhibitor can be dasatinib or saracatinib. One of the first polypeptide, the second polypeptide, and the third polypeptide can be a TRAIL polypeptide, and one of the first inhibitor, the second inhibitor, and the third inhibitor can inhibit expression or activity of the TRAIL polypeptide. One of the first inhibitor, the second inhibitor, and the third inhibitor can be an siRNA molecule that can inhibit expression of the TRAIL polypeptide. One of the first inhibitor, the second inhibitor, and the third inhibitor can inhibit activity of the TRAIL polypeptide. The vesicle can include a fourth inhibitor located within the vesicle, where the fourth inhibitor can inhibit expression or activity of a fourth polypeptide. The first inhibitor can inhibit expression or activity of said YAP polypeptide, the second inhibitor can inhibit expression or activity of said TAZ polypeptide, the third inhibitor can inhibit expression or activity of said LCK polypeptide, and the fourth can inhibit expression or activity of said TRAIL polypeptide. The vesicle also can include an imaging agent. The imaging agent can be a fluorophore, a fluorescent dye, or a radionucleotide. The cancer cell can be located within a liver, gall bladder, small intestine, or bile duct of a mammal. The mammal can be a human. The cancer cell can be a liver cancer cell, a gall bladder cancer cell, a small intestine cancer cell, or a bile duct cancer cell. The cancer cell can be a cholangiocarcinoma cancer cell. The molecule can be an epithelial cell adhesion molecule (EpCAM) polypeptide, an EPH receptor A2 (EPHA2) polypeptide, an AXL polypeptide, a G Protein-coupled receptor class C group 5 member A (GPRC5a) m polypeptide, a GPRC5c polypeptide, an epidermal growth factor receptor (EGFR) polypeptide, a cytokeratin-19 (CK19) polypeptide, an osteopontin (SPP1) polypeptide, or a Sox9 polypeptide. The targeting moiety can be an antibody. The targeting moiety can be a nucleic acid aptamer. The nucleic acid aptamer can be an EpCAM aptamer, an AXL aptamer, an EGFR aptamer, a CK19 aptamer, a SPP1 aptamer, or a Sox9 aptamer. The nucleic acid aptamer can be an RNA aptamer. The nucleic acid aptamer can be a DNA aptamer. The longest diameter of the vesicle can be 100 nm to 500 nm. The longest diameter of the vesicle can be 200 nm to 400 nm. The nucleic acid aptamer can be an EpCAM aptamer having the nucleic acid sequences set forth in any one of SEQ ID NOs: 31-39 and 42-52.

In another aspect, this document features vesicles including an inhibitor located within the vesicle and a targeting moiety located on a surface of the vesicle, where the inhibitor can inhibit expression or activity of a LCK polypeptide or a TRAIL polypeptide, where the targeting moiety can bind to a molecule present on the surface of a cancer cell, and where the vesicle has a longest diameter of 5 nm to 500 nm. The inhibitor can inhibit expression or activity of the LCK polypeptide. The inhibitor can be an siRNA molecule that can inhibit expression of the LCK polypeptide. The inhibitor can inhibit activity of the LCK polypeptide. The inhibitor can be dasatinib or saracatinib. The vesicle can include a second inhibitor located within the vesicle. The second inhibitor can inhibit expression or activity of a YAP polypeptide, a TAZ polypeptide, or said TRAIL polypeptide. The second inhibitor can inhibit expression or activity of a YAP polypeptide. The second inhibitor can inhibit expression or activity of a TAZ polypeptide. The second inhibitor can inhibit expression or activity of a TRAIL polypeptide. The vesicle can include a second inhibitor located within the vesicle and a third inhibitor located within the vesicle. The second inhibitor and the third inhibitor can inhibit expression or activity of a YAP polypeptide, a TAZ polypeptide, or a TRAIL polypeptide. The vesicle can include a second inhibitor located within the vesicle, a third inhibitor located within the vesicle, and a fourth inhibitor located within the vesicle. The second inhibitor can inhibit expression or activity of a YAP polypeptide, the third inhibitor can inhibit expression or activity of a TAZ polypeptide, and the fourth inhibitor can inhibit expression or activity of a TRAIL polypeptide. The inhibitor can inhibit expression or activity of a TRAIL polypeptide. The inhibitor can be an siRNA molecule that can inhibit expression of a TRAIL polypeptide. The inhibitor can inhibit activity of a TRAIL polypeptide. The vesicle can include a second inhibitor located within the vesicle. The second inhibitor can inhibit expression or activity of a YAP polypeptide, a TAZ polypeptide, or a LCK polypeptide. The second inhibitor can inhibit expression or activity of a YAP polypeptide. The second inhibitor can inhibit expression or activity of a TAZ polypeptide. The second inhibitor can inhibit expression or activity of a LCK polypeptide. The vesicle can include a second inhibitor located within the vesicle and a third inhibitor located within the vesicle. The second inhibitor and the third inhibitor can inhibit expression or activity of a YAP polypeptide, a TAZ polypeptide, or a LCK polypeptide. The vesicle can include a second inhibitor located within the vesicle, a third inhibitor located within the vesicle, and a fourth inhibitor located within the vesicle. The second inhibitor can inhibit expression or activity of a YAP polypeptide, the third inhibitor can inhibit expression or activity of a TAZ polypeptide, and the fourth inhibitor can inhibit expression or activity of a LCK polypeptide. The vesicle can include an imaging agent. The imaging agent can be a fluorophore, a fluorescent dye, or a radionucleotide. The cancer cell can be located within a liver, gall bladder, small intestine, or bile duct of a mammal. The mammal can be a human. The cancer cell can be a liver cancer cell, a gall bladder cancer cell, a small intestine cancer cell, or a bile duct cancer cell. The cancer cell can be a cholangiocarcinoma cancer cell. The molecule can be an EpCAM polypeptide, an EPHA2 polypeptide, an AXL polypeptide, a GPRC5a polypeptide, a GPRC5c polypeptide, an EGFR polypeptide, a CK19 polypeptide, an SPP1 polypeptide, or a Sox9 polypeptide. The targeting moiety can be an antibody. The targeting moiety can be a nucleic acid aptamer. The nucleic acid aptamer can be an EpCAM aptamer, an AXL aptamer, an EGFR aptamer, a CK19 aptamer, a SPP1 aptamer, or a Sox9 aptamer. The nucleic acid aptamer can be an RNA aptamer. The nucleic acid aptamer can be a DNA aptamer. The longest diameter of the vesicle can be 100 nm to 500 nm. The longest diameter of the vesicle can be 200 nm to 400 nm. The nucleic acid aptamer can be an EpCAM aptamer having the nucleic acid sequences set forth in any one of SEQ ID NOs: 31-39 and 42-52.

In another aspect, this document features methods for cancer. The methods can include, or consist essentially of, administering a composition comprising a vesicle described herein to a mammal having cancer, where the number of cancer cells within the mammal is reduced following the administering of the composition. The mammal can be a human. The cancer cells can be liver cancer cells, gall bladder cancer cells, small intestine cancer cells, or bile duct cancer cells. The cancer can be a cholangiocarcinoma.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Human cholangiocarcinoma (CCA) cells express TRAIL and myeloid-derived suppressor cells (MDSCs) express TRAIL-R 1-4. UMAP plots from single cell RNA (scRNA sequencing of resected human CCAs (n=10). FIG. 1A) CCA cells express TRAIL (TNFSF10). FIG. 1B) MDSCs express TRAIL-R1 (TNFRSF10A), TRAIL-R2 (TNFRSF10B), TRAIL-R3 (TNFRSF10C), and TRAIL-R4 (TNFRSF10D).

FIGS. 2A-2B. Trail-r^{-/-} mice have significant reduction in tumor burden. Average tumor weights in milligrams (mg) and representative photomicrographs of wild type (WT) and Trail-r^{-/-} mice 4 weeks following implantation of murine SB cells (FIG. 2A) or KrasG12Dp53L/L into mouse livers (FIG. 2B). *, P<. 05; ***, P<001.

FIG. 3. Trail-r^{-/-} mouse tumors have enhanced cytotoxic T lymphocyte (CTL) infiltration and function but no change in CTL proliferation or apoptosis. Flow cytometry analysis of mouse tumors in WT and Trail-r^{-/-} mice 4 weeks following SB implantation. Lymphocytes were stained for CD3, CD8, CD11a, granzyme B, Annexin V, 7AAD, or Ki67. ***, P<. 001.

FIGS. 4A-4C. Trail-r^{-/-} mouse tumors have a significant reduction in granulocyte-like MDSCs (G-MDSCs) and monocytic MDSCs (M-MDSCs) as well as decreased MDSC proliferation. Flow cytometry analysis of mouse tumors in WT and Trail-r^{-/-} mice 4 weeks following SB implantation. Cells were stained for CD11b, F480, Ly6C, Ly6G, and Ki67. *, P<0.05; **, P<0.01.

FIGS. 5A-5C. Cancer cell restricted deletion of Trail results in a significant reduction in tumors in WT mice whereas deletion of Trail-r augments tumor growth. FIG. 5A) Murine CCA cells with CRISPR-cas9-mediated knockdown of Trail (SB-Trail^{-/-}), Trail-r (SB-Trail-r^{-/-}) or non-target (SB-NT) were generated. FIGS. 5B and 5C) SB-NT, SB-SB-Trail^{-/-}, and SB-Trail-r^{-/-} were implanted into livers of WT mice. After 4 weeks of tumor growth, mice were sacrificed and tumor were characterized. *, P<0.05.

FIG. 6. Genetic deletion of Trail-r results in a significant reduction in MDSC proliferation and PD-L1 expression. MDSCs differentiated from bone marrow (BM) of WT or Trail-r^{-/-} mice were cocultured with murine tumor cells for 24 hours. Following coculture, MDSCs were harvested, stained with fluorescent antibodies, and analyzed by flow cytometry to assess their proliferation (Ki67 expression; FIG. 6A) and immunosuppressive function (PD-L1 expression; FIG. 6B). **, P<0.01; ****, p<0.0001.

FIG. 7. TRAIL-TRAIL-R activates NF-κB signaling in MDSCs. Representative immunofluorescence for the NF-κB subunit p65 on wild-type (WT) or Trail-r^{-/-} MDSCs incubated with murine CCA cells.

FIG. 8. ROCK inhibition decreases PD-L1 expression on MDSCs. MDSCs differentiated from BM of WT were cocultured with murine tumor cells and incubated with a ROCK inhibitor (Y-27632). PD-L1 expression was assessed in MDSCs via flow cytometry **, P<0.01.

FIGS. 9A-9C. Therapeutic effect of EPCAM targeting (ET) nanovesicles in vitro and in vivo. FIG. 9A) Murine CCA cells with incubated with WT nanovesicles loaded with a fluorescent dye. FIG. 9B) Immunoblot for TRAIL in murine CCA cells following incubation with ET-nanovesicles loaded with siRNA for TRAIL or control (siCtrl). FIG. 9C) Representative immunofluorescence images of Alexa647-3wj decorated siRNA loaded PKH67 labeled ET nanovesicles in murine CCA tumor (Hoechst).

FIGS. 10A-10B. High-dimensional imaging of human CCA. FIG. 10A) Multiplexed immunofluorescence (MxIF) image of a human CCA tumor specimen depicting tumor cells and immune cells. FIG. 10B) MxIF overlay image depicting TRAIL⁻ tumor cells.

FIGS. 11A-11I. Characterization of YAP, TAZ, and LCK expression in CCA. FIG. 11A) Expression of YAP1, TAZ, and LCK mRNA in TCGA database. FIG. 11B) Expression of YAP1, TAZ, and LCK mRNA in 102 CCA samples and 59 surrounding liver tissues in GEO database (GSE26566). FIG. 11C) The correlation between YAP1 mRNA and downstream targets of YAP, including CYR61 and CTGF in 102 CCA samples (upper panel) derived from GEO database. FIG. 11D) The correlation between YAP1 mRNA and downstream targets of YAP, including CYR61 and CTGF in 59 surrounding liver tissues derived from GEO database. FIG. 11E) Representative images showing human CCA tumors (Liv31) were implanted in the liver of male NOD/SCID mice. FIG. 11F) Immunoblot demonstrating YAP, pYAP, TAZ, LCK, and Actin levels in normal human cholangiocyte (NHC) cells, HuCCT1 cells, and Liv31 PDX tumor tissue. FIGS. 11G-11I) Immunoblot analysis of SB1 cells showed elevated YAP, TAZ, and LCK expression in total lysate and nucleus comparing to normal murine cholangiocytes (603B) cells.

FIGS. 12A-12L. Characterization of EPCAM expression in CCA. FIG. 12A) Immunohistochemical staining of EpCAM in patients with cholangiocarcinoma. FIG. 12B) Comparing EPCAM mRNA expression between CCA and normal liver tissues in TCGA database analysis. FIGS. 12C-12D) Immunoblot analysis of whole lysates derived from HuCCT-1 cells and NHC cells showed elevated EpCAM expression in human cholangiocarcinoma cells (FIG. 12C). Relative protein expression of EpCAM in HuCCT-1 cells and NHC cells was quantified using ImageJ (FIG. 12D). FIGS. 12E-12F) Immunoblot analysis of whole lysates derived from NHC cells and Liv31 bulk tumor showed elevated EpCAM expression in human cholangiocarcinoma (FIG. 12E). Relative protein expression of EpCAM was quantified using ImageJ (FIG. 12F). FIG. 12G) EpCAM expression was assessed by immunofluorescence in HuCCT-1 cells. Scale bars=50 μm. FIG. 12H) EpCAM expression was assessed by immunofluorescence in NHC cells. Scale bars=50 μm. FIG. 12I) EpCAM expression was assessed by immunofluorescence in Liv31 orthotopic tumor section. Scale bars=100 μm. FIG. 12J) EpCAM expression was assessed by immunofluorescence in SB1 cells. Scale bars=50 μm. FIG. 12K) EpCAM expression was assessed by immunofluorescence in 603B cells. Scale bars=50 μm. FIG. 12L) Immunoblot analysis of whole lysates derived from SB1 cells and 603B cells showed elevated EpCAM expression in murine cholangiocarcinoma cells (left panel). And relative protein expression of EpCAM were quantified in SB1 cells and 603B cells using ImageJ (right panel).

FIGS. 13A-13J. Cellular uptake of nanovesicles and bio-distribution of nanovesicles in vivo. FIG. 13A) Schematic illustration of therapeutic milk-derived nanovesicles decorated with aptamer (EpDT3) which targets EpCAM protein. FIGS. 13B-13C) Fresh SB1 tumor and normal adjacent liver were cut into 3×3×3 mm cubes and placed in 96 well plates. Samples were incubated for 24 hours with 2E+12 nanovesicles with Alexa647 labeled aptamer and stained for flow cytometry. FIG. 13D) Nanovesicle injection timing for C57BL/6 mice bearing subcutaneous and orthotopic SB1 tumors for biodistribution analysis. FIG. 13E) Representative images of subcutaneous and orthotopic SB1 tumors. Immunochemistry staining of CK19 for SB1 tumor and adjacent normal liver tissues. FIGS. 13F-13G) 6 hours after the tail vein injection of 2E+12 nanovesicles, fresh tissue samples were taken out immediately and embedded in OCT, and then frozen sectioning were performed. Alexa647 positive cells were quantified in 10 high powered fields. Representative images of gross tissues, H&E staining and frozen section were shown in the left. ** p<0.01, **** p<0.0001. FIG. 13H) Representative images of frozen sectioning of CCA tumors and adjacent normal liver tissues showing the delivery of nanovesicles in C57BL/6 mice bearing orthotopic SB1 tumors. FIG. 13I) Representative images showing human CCA tumors (Liv31) were implanted in the liver of male NOD/SCID mice. H&E, CK19, and SOX9 staining of Liv31 tumors. FIG. 13J) Representative images of frozen sectioning of CCA tumors and adjacent normal liver tissues showing the delivery of nanovesicles in NOD/SCID mice bearing orthotopic Liv31 tumors.

FIGS. 14A-14I. Effects mediated by nanovesicles loaded with siRNAs in vitro. FIG. 14A) Immunoblot analysis of whole lysates derived from SB1 cells showed knockdown of YAP protein mediated by nanovesicles loaded with siYAP. FIG. 14B) Immunoblot analysis of whole lysates derived from SB1 cells showed knockdown of TAZ protein mediated by nanovesicles loaded with siTAZ. FIG. 14C) Immunoblot analysis of whole lysates derived from SB1 cells showed knockdown of LCK protein mediated by nanovesicles loaded with siLCK. FIG. 14D) qRT-PCR analysis showing the knockdown of YAP1, TAZ and LCK mRNA level mediated by nanovesicles loaded with siYAP, siTAZ or siLCK separately, by and nanovesicles loaded with siTriple (siYAP, siTAZ and siLCK combined) in SB1 cells. FIG. 14E) Immunofluorescent staining comparing YAP, TAZ and LCK expression in SB1 cells treated with nanovesicles loaded with siNC and the combination of siYAP, siTAZ and siLCK. FIG. 14F) Immunofluorescent staining showing YAP expression in SB1 cells treated with nanovesicles loaded with siNC, siYAP and the combination of siYAP, siTAZ and siLCK (left panel). YAP mean fluorescence intensity (MFI) was plotted as a violin plot (right panel). The YAP expression in nucleus is lower in treatment with the combination of siYAP, siTAZ and siLCK compared to the siYAP alone. FIG. 14G) Representative PI and Hoechst 33342 staining in SB1 cells treated with nanovesicles loaded with siNC, siYAP and siTriple (siYAP, siTAZ and siLCK combined) in SB1 cells. FIG. 14H) Clonal formation assays were performed to analyze the proliferation of SB1 cells treated with nanovesicles loaded with siNC, siTriple (siYAP, siTAZ and siLCK combined), Gem/Cis or siTriple combined with Gem/Cis. FIG. 14I) Relative caspase 3/7 activity assays were performed to detect the apoptosis of SB1 cells treated with nanovesicles loaded with siNC, siTriple (siYAP, siTAZ and siLCK combined), Gem/Cis or siTriple combined with Gem/Cis.

FIGS. 15A-15F. Nanovesicle-mediated knockdown effects in vivo. FIG. 15A) Schedule of nanovesicles injection in C57BL/6 mice bearing both subcutaneous and orthotopic SB1 tumors. FIGS. 15B-15C) qRT-PCR analysis showing the knockdown of YAP1, TAZ and LCK mRNA level mediated by nanovesicles loaded with siYAP, siTAZ and siLCK in orthotopic SB1 tumors. The relative quantification (FIG. 15B) and fold changes (FIG. 15C) of mRNA levels were calculated. FIG. 15D) Representative YAP immunofluorescence staining and representative images of H&E in SB1 tumor as well as corresponding adjacent normal liver tissue sections derived from mice treated with nanovesicles loaded with siNT or siTriple (150 μm scale bar). Dotted line indicates the borderlines of cholangiocarcinoma and adjacent normal liver tissue sections. FIG. 15E) Representative YAP immunofluorescence staining and representative images of H&E in Liv31 tumor as well as adjacent normal liver tissue sections derived from mice treated with nanovesicles loaded with siNT or siTriple (150 μm scale bar). FIG. 15F) The knockdown effects on nuclear YAP and TAZ expression in Liv31 tumors harvested from representative mice treated with nanovesicles loaded with siNT or siTriple, were evaluated by immunoblot assays.

FIGS. 16A-16J. Nanovesicles loaded with siYAP, siTAZ and siLCK enhanced the efficacy of chemotherapy in CCA models. FIG. 16A) Schedule of SB1 tumor implantation and treatments of Gem/Cis, nanovesicles loaded with siRNAs and normal saline in C57BL/6 mice. Treatments were initiated three weeks after SB1 cell orthotopic implantation. C57BL/6 mice were treated with Gem (15 mg/kg)/Cis (1 mg/kg) every three days, 2E+12 nanovesicles loaded with 5 μM siYAP, siTAZ and siLCK every two days, or normal saline. FIG. 16B) Representative images from orthotopic SB1 tumor harvested from the indicated treatments. FIG. 16C) Tumor weights of orthotopic SB1 tumor harvested from the indicated treatments. FIG. 16D) Representative images from orthotopic SB1 tumor harvested from the indicated treatments. FIG. 16E) Tumor weights of orthotopic SB1 tumor harvested from the indicated treatments. FIG. 16F) Tumor weights of orthotopic SB1 tumor harvested from the indicated treatments in combined analysis. FIG. 16G) Schematic representation of the therapy schedule for the indicated treatments in NOD/SCID mice bearing Liv31 tumors. Treatments were initiated four weeks after PDX tumor (Liv31) orthotopic implantation. NOD/SCID mice were treated with Gem (4 mg/kg)/Cis (1 mg/kg) two times a week, 2E+12 nanovesicles loaded with 5 M siYAP, siTAZ and siLCK, or siNC every two days. FIG. 16H) Representative images from orthotopic Liv31 tumors harvested from the indicated treatments. FIG. 16I) Tumor weights of orthotopic Liv31 tumor harvested from the indicated treatments. FIG. 16J) Representative images following cleaved caspase-3 and ki67 immunochemistry staining in tumors from C57BL/6 mice bearing orthotopic SB1 tumor with different treatment, with quantification of Ki67, cleaved caspase-3-positive cells (right panel).

FIGS. 17A-17L. Nanovesicles loaded with siYAP, siTAZ and siLCK transformed the lymphocyte landscape of the tumor microenvironment (TME) in CCA models. FIG. 17A) Representative images from orthotopic SB1 tumor harvested from the indicated treatments. FIG. 17B) Percentage of CD8 T cells (CD45⁺CD3⁺CD8⁺) in total leukocytes (CD45⁺) in SB1 tumors treated with nanovesicles loaded with siNT and siTriple (n=7). FIG. 17C) Percentage of reactive T cells (CD45⁺CD3⁺CD8⁺CD11a⁺) in total leukocytes (CD45⁺) in SB1 tumors treated with nanovesicles loaded with siNT and siTriple (n=7). FIG. 17D) Percentage of reactive T cells (CD45⁺CD3⁺CD8⁺CD11a⁺) in total lymphocytes (CD45⁺CD3⁺) in SB1 tumors treated with nanovesicles loaded with siNT and siTriple (n=7). FIG. 17E) Representative flow plots show expression of CD11a in CD8 T cells. FIG. 17F) Percentage of reactive T cells (CD45⁺CD3⁺CD8⁺CD11a⁺) in CD8 T cells in SB1 tumors treated with nanovesicles loaded with siNT and siTriple (n=7). FIG. 17G) Representative flow plots show expression of PD-1 in reactive CD8 T cells (CD45⁺CD3⁺CD8⁺CD11a⁺). FIG. 17H) Percentage of PD-1⁺reactive T cells (CD45⁺CD3⁺CD8⁺CD11a′PD-1⁺) in CD8 T cells in SB1 tumors treated with nanovesicles loaded with siNT and siTriple (n=7). FIG. 17I) Representative flow plots show expression of GranzymeB in reactive CD8 T cells (CD45⁺CD3⁺CD8⁺CD11a⁺). FIG. 17J) Percentage of GranzymeB reactive T cells (CD45⁺CD3⁺CD8⁺CD11a GranzymeB⁺) in CD8 T cells in SB1 tumors treated with nanovesicles loaded with siNT and siTriple (n=7). FIGS. 17K-17L) Percentage of NK cells of total leukocytes (CD45⁺) in SB1 tumors treated with nanovesicles loaded with siNT and siTriple (n=7). Fluorescence Minus One (FMO) controls were used for each independent experiment to establish gates.

FIG. 18. Characterization of EPCAM expression in CCA. EpCAM expression was assessed by immunofluorescence in SB1 orthotopic tumor section. Scale bars=100 μm. FIGS. 19A-19I. Effects mediated by nanovesicles loaded with siRNAs in vitro.

FIG. 19A) Immunoblot analysis of whole lysates derived from HuCCT1 cells showed knockdown of YAP protein mediated by nanovesicles loaded with siYAP. FIG. 19B) Immunoblot analysis of whole lysates derived from HuCCT1 cells showed knockdown of TAZ protein mediated by nanovesicles loaded with siTAZ FIG. 19C) Immunoblot analysis of whole lysates derived from HuCCT1 cells showed knockdown of LCK protein mediated by nanovesicles loaded with siLCK. FIG. 19D) qRT-PCR analysis comparing the mRNA expression of YAP1 downstream targets, including CTGF and CYR61, anti-apoptotic targets, including MCL1, Bcl-xL and Bcl-2 in SB1 cells treated with nanovesicles loaded with siNC or siYAP in HuCCT1 cells. FIG. 19E) qRT-PCR analysis comparing the mRNA expression of YAP1 downstream targets, including CTGF and CYR67 treated with nanovesicles loaded with siNC or siLCK in HuCCT1 cells. FIG. 19F) Immunoblot analysis of nuclear lysates derived from HuCCT1 cells showed knockdown of nuclear YAP and TAZ protein mediated by nanovesicles loaded with siTriple (siYAP, siTAZ and siLCK combined). FIGS. 19G-19H) Flowcytometry analysis was performed to analyze the apoptosis of HuCCT1 cells treated with nanovesicles loaded with siYAP or siLCK. FIG. 211) Relative caspase 3/7 activity assays were performed to detect the apoptosis of HuCCT1 cells treated with nanovesicles loaded with siYAP or siLCK.

FIGS. 20A-20D. Nanovesicles-mediated knockdown effects in vivo. FIGS. 20A-20B) qRT-PCR analysis showing the knockdown of YAP1, TAZ and LCK mRNA level mediated by nanovesicles loaded with siYAP, siTAZ and siLCK in subcutaneous SB1 tumors. The relative quantification (FIG. 20A) and fold changes (FIG. 20B) of mRNA levels were calculated. FIGS. 20C-20D) qRT-PCR analysis showing the knockdown of YAP1, TAZ and LCK mRNA level mediated by nanovesicles loaded with siYAP, siTAZ and siLCK in corresponding normal liver tissues. The relative quantification (FIG. 20C) and fold changes (FIG. 20D) of mRNA levels were calculated.

FIGS. 21A-21C. Nanovesicles loaded with siYAP, siTAZ and siLCK enhanced the efficacy of chemotherapy in CCA models. FIGS. 21A-21B) Tumor weights of orthotopic SB1 tumor harvested from the indicated treatments. FIG. 21C) Tumor weights of orthotopic Liv31 tumor harvested from the indicated treatments.

FIGS. 22A-22F. Docking and stability of EpCAM targeting aptamers. FIGS. 22A-22C) Protein-ligand molecular docking evaluation of aptamers to the 3D structure of the EpCAM extracellular domain (EpEX) (PDB: 6107) for DNA aptamers (FIG. 22A) PL-d01, (FIG. 22B) PL-d02 and RNA aptamer (FIG. 22C) PL-95. Docking was performed with 70,000 iterations and 30 poses, ranked by their PIPER pose energy score were generated. The pose with the lowest PIPER score for each docking experiment is shown. FIGS. 22D-22F) Molecular dynamics simulations of (FIG. 22D) PL-d01, (FIG. 22E) PL-d02 and (FIG. 22F) PL-95 interactions with the extracellular domain of EpCAM monomer for 200 ns at normal pressure and temperature. The resulting root mean square deviations (RMSD) plots were generated to assess aptamer-protein complex stability over time.

FIG. 23. Binding sites of DNA (top panel) and RNA (bottom panel) aptamers on EpCAM. The EpCAM residues that were in contact with the aptamers PL-d01, PL-d02 and PL-95 or with other DNA aptamers (Asic, Epp166, Tepp) and RNA aptamers (EpDT3) were identified from molecular dynamics simulations of aptamer binding with the EpCAM extracellular residues performed using Schrodinger Maestro software. The regions on EpCAM where the aptamers interacted during the molecular dynamics simulations are indicated for each aptamer.

FIGS. 24A-24E. Decoration efficiency of milk derived nanovesicles (MNVs) with DNA aptamers. 2.00×10¹¹ DiO stained MNVs were decorated with 0.01, 0.1, or 1 μM of CY5-PLd01-TEGCholesterol or CY5-PLd02-TEGCholesterol to generate targeted MNVs (T-MNVs). FIG. 24A) ImageStream flow cytometer to detect aptamer labels on MNVs. FIGS. 24B-24E) Cy5 fluorescence intensity was used to quantify the decoration efficiency of PL-d01 and PL-d02 decorated MNVs.

FIGS. 25A-25B. Cell binding affinity of aptamers. FIG. 25A) HuCCT1 cholangiocarcinoma cells or HL-60 leukemia cells were incubated with 250 nM aptamer, then washed and incubated with AlexaFluor647 conjugated streptavidin and analyzed using a Novocyte flow cytometer. 30,000 events were acquired. FIG. 25B) The background corrected median fluorescence intensity of AF647 was used for comparison of aptamer binding across cells

FIG. 26. Quantification of recovered molecules in each round. Quantification of bound aptamers in each selection round was performed by qPCR. To enrich the aptamers that specifically target human cholangiocarcinoma HuCCT1 cells, normal cholangiocytes (NHC cells) and hepatocytes (Hu1545 cells) were alternatively employed as negative selection cells. The amount of recovered molecules was increased along with the number of rounds.

FIG. 27. Enrichment of aptamers throughout selection. 11 rounds of selection were performed with an 80 nt DNA oligonucleotide library (random region n=40 nt) targeting HuCCT1 cells. The negative selection step was performed with normal cholangiocyte cell line, NHC (for 0, 3, 5, 7, 9 and 11^{th} round) and normal hepatocyte cell line, Hu1545 (for 2, 4, 6, 8, 10^{th} round).

FIGS. 28A-28B. Aptamer binding activity in cell culture. FIG. 28A) Quantification of aptamer binding detected by imaging. Aptamer candidates (LJM-7122 through LJM-7130) and negative controls (LJM-7131 and LJM-7132) are synthesized with 3′ biotin modification and detected by staining with AlexaFluor647-labeled streptavidin. Aptamer is incubated on cells at 50 nM in selection buffer before fixation and streptavidin post-staining. Data points represent individual fields taken across multiple wells. FIG. 28B) Representative images of aptamer binding.

FIG. 29. Aptamer binding activity in cell culture by quantitative PCR. Aptamer candidates (LJM-7122 through LJM-7130) and negative controls (LJM-7131, LJM-7132 and #1library) were incubated on human cholangiocarcinoma HuCCT1 cells at 100 nM in selection buffer for 30 minutes followed by washing out unbound aptamers. Cells were boiled and the collected supernatant were subjected to qPCR. Data points replicated wells. *** p<0.001, **** p<0.001.> #7122, #7125, #7127, #7128, #7130 aptamers have significantly higher binding capacity to HuCCT1 cells among the nine candidates.

FIGS. 30A-30C. Aptamer binding to multiple human cell lines in cell culture. Aptamer candidates and matched negative controls were synthesized with 3′ biotin modification and detected by staining with AlexaFluor647-labeled streptavidin. Aptamer was incubated on cells at 50 nM in selection buffer before fixation and streptavidin post-staining. Data points represent individual fields taken across multiple wells. FIG. 30A) Binding of LJM-7122 and scrambled negative control LJM-7131. FIG. 30B) Binding of LJM-7127 and scrambled negative control LJM-7162. FIG. 30C) Binding of LJM-7130 and scrambled negative control LJM-7163. Hu1545 cells display elevated background streptavidin binding due to high levels of endogenous biotin.

FIG. 31. Aptamer #7122 binding to multiple human cell lines in cell culture. Aptamer #7122 and control aptamer (#7131) were incubated on multiple human cell lines at 100 nM in selection buffer for 30 minutes followed by washing out unbound aptamers. HuCCT1 and RBE cells are human cholangiocarcinoma cell line, and NHC and Hu1545 cells are normal human cholangiocyte, normal human hepatocyte, respectively. Cells were boiled and the collected supernatant were subjected to qPCR. Data points replicated wells. *** p<0.001, **** p<0.001, ns, non-significant.>#7122 aptamer selectively binds to cholangiocyte (both CCA cells and normal cells).

FIGS. 32A-32B. Aptamer binding to mouse cell lines in cell culture. Aptamer candidates and matched negative controls were synthesized with 3′ biotin modification and detected by staining with AlexaFluor647-labeled streptavidin. Aptamer was incubated on cells at 50 nM in selection buffer before fixation and streptavidin post-staining. Data points represent individual fields taken across multiple wells. FIG. 32A) Binding of top aptamers and matched negative controls on FAC cells. FIG. 32B) Binding of top aptamers and matched negative controls on SB1 cells.

FIG. 33. Aptamer binding to mouse cell lines in cell culture. Aptamers (#7122, #7127 and #7130) and respective control aptamers (#7131, #7162 and #7163) were incubated on mouse cholangiocarcinoma cells at 100 nM in selection buffer for 30 minutes followed by washing out unbound aptamers. FAK and SB1 cells are both mouse cholangiocarcinoma cell line. Cells were boiled and the collected supernatant were subjected to qPCR. Data points replicated wells. *** p<0.001, **** p<0.001. These aptamer candidates bound both human CCA cells and mouse CCA cells.

FIGS. 34A-34B. Truncations of aptamer LJM-7122 reduced binding activity. FIG. 34A) Secondary structure of LJM-7122 (SEQ ID NO:53) in selection buffer conditions predicted by mfold. Regions removed in truncated aptamer versions are indicated by arrows. FIG. 34B) Variable 5′ and 3′ terminal sequences were removed from full-length aptamer LJM-7122 and cell binding activity was detected by qPCR. qPCR primers were adapted according to terminal truncations.

FIGS. 35A-35B. SB1 cholangiocarcinoma cells were treated in vivo with EpCAM-aptamer targeted milk derived nanovesicles for 48 hours. Cell death was evaluated by propidium iodide (PI) staining with a Hoechst counterstain to facilitate counting. FIG. 35A) Single targets included siRNA against WWTR1 or LCK. Triple targets included siRNA against the WWTR1, LCK, and YAP. FIG. 35B) Single targets included siRNA against YAP, LCK, and TAZ (tafazzin). Triple targets included siRNA against TAZ, LCK, and YAP. Control vesicles loaded with a non-targeting siRNA. * p<0.05 *** p<0.001 **** P<0.0001. ns=non-significant.

FIGS. 36A-36D. Nanovesicles loaded with siRNA to PD-L1 enhanced the efficacy of chemotherapy in CCA models. FIG. 36A) Schedule of SB 1 tumor implantation and treatments of gemcitabine, nanovesicles loaded with siRNA to PD-L1 and normal saline in C57BL/6 mice. Treatments were initiated fifteen days after SB1 cell orthotopic implantation and were administered every three days for a total of five doses. C57BL/6 mice were treated with 2E+12 nanovesicles loaded with 5 μM siRNA to PD-L1 (MNV-PD-L1) or a non-targeting control inhibitor (MNV-NC), with or without 30 mg/kg gemcitabine (Gem) every three days for a total of five doses, or normal saline (control). FIG. 36B) Representative images from orthotopic SB1 tumor harvested from the indicated treatments. FIG. 36C) Weights of tumor-bearing whole livers from mice receiving the indicated treatments. FIG. 36D) Tumor weights of orthotopic SB1 tumor harvested from the indicated treatments. * p<0.01 between groups.

FIGS. 37A-37B. Silencing effects mediated by siRNAs to PD-L1 in vitro after cell transfection or delivery using nanovesicles. FIG. 37A) Immunoblot analysis of whole lysates derived from HuCCT1 cells showed knockdown of PD-L1 protein mediated by two different siRNA inhibitors to PD-L1, siRNA #1 and siRNA #2 following transfection of HuCCT1 cells using lipofectamine 2000. The right panel shows quantitative data (mean and SD) from three separate determinations, * p<0.0001. FIG. 37B) Immunoblot analysis of whole lysates derived from HuCCT1 cells showed knockdown of PD-L1 protein mediated by nanovesicles loaded with siRNA #1 and siRNA #2 to PD-L1. The right panel shows quantitative data (mean and SD) from three separate determinations, * p<0.0001.

### DETAILED DESCRIPTION

This document provides methods and materials for treating cancer. For example, this document provides vesicles (e.g., nanovesicles) designed to deliver one or more (e.g., one, two, three, or more) inhibitors to a cancer cell within a mammal (e.g., a human) having cancer to treat the mammal. In some cases, a vesicle provided herein can be designed to include (a) a targeting moiety located on a surface of the vesicle and (b) one or more (e.g., one, two, three, or more) inhibitors located within the vesicle. In some cases, a mammal (e.g., a human) having cancer (e.g., cholangiocarcinoma) can be administered one or more vesicles provided herein (e.g., one or more vesicles that include (a) a targeting moiety located on a surface of the vesicle(s) and (b) one or more (e.g., one, two, three, or more) inhibitors located within the vesicle(s)) to treat the mammal's cancer.

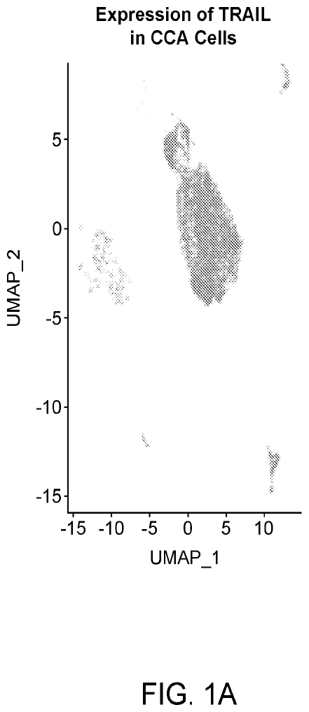

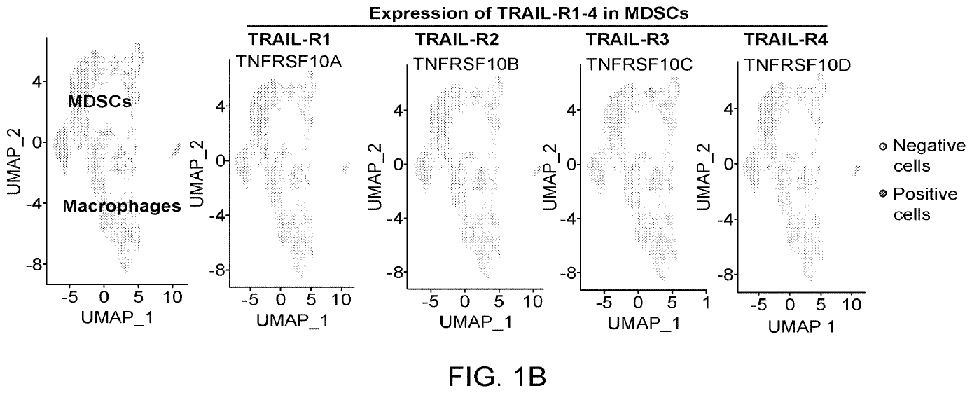

A vesicle provided herein (e.g., a vesicle having (a) a targeting moiety located on a surface of the vesicle and (b) one or more (e.g., one, two, three, or more) inhibitors located within the vesicle) can be any appropriate type of vesicle. In some cases, a vesicle provided herein can be designed from a synthetic vesicle. In some cases, a vesicle provided herein can designed from a naturally occurring vesicle. Examples of vesicles that can be designed to have (a) a targeting moiety located on a surface of the vesicle and (b) one or more (e.g., one, two, three, or more) inhibitors located within the vesicle, and can be used as described herein include, without limitation, milk-derived nanovesicles (e.g., milk exosomes), extracellular vesicles, liposomes, lipid nanoparticles, and protein-based nanoparticles. In some cases, a vesicle described elsewhere (see, e.g., Elkhoury et al., Pharmaceutics, 12:849 (2020) at, for example, FIG. 1 and Table 1) can be designed to include (a) a targeting moiety located on a surface of the vesicle and (b) one or more (e.g., one, two, three, or more) inhibitors located within the vesicle.

A vesicle provided herein (e.g., a vesicle having (a) a targeting moiety located on a surface of the vesicle and (b) one or more (e.g., one, two, three, or more) inhibitors located within the vesicle) can be any appropriate size. In some cases, a vesicle provided herein can have a longest dimension (e.g., a longest diameter) of from about 1 nm to about 1000 nm (e.g., from about 5 nm to about 500 nm, from about 10 nm to about 500 nm, from about 25 nm to about 500 nm, from about 50 nm to about 500 nm, from about 100 nm to about 500 nm, from about 150 nm to about 500 nm, from about 200 nm to about 500 nm, from about 250 nm to about 500 nm, from about 300 nm to about 500 nm, from about 400 nm to about 500 nm, from about 5 nm to about 450 nm, from about 5 nm to about 400 nm, from about 5 nm to about 350 nm, from about 5 nm to about 300 nm, from about 5 nm to about 250 nm, from about 5 nm to about 200 nm, from about 5 nm to about 150 nm, from about 5 nm to about 100 nm, from about 5 nm to about 50 nm, from about 5 nm to about 25 nm, from about 10 nm to about 450 nm, from about 10 nm to about 400 nm, from about 20 nm to about 400 nm, from about 50 nm to about 400 nm, from about 100 nm to about 400 nm, from about 10 nm to about 300 nm, from about 20 nm to about 300 nm, from about 50 nm to about 300 nm, from about 100 nm to about 300 nm, from about 10 nm to about 200 nm, from about 20 nm to about 200 nm, from about 50 nm to about 200 nm, from about 100 nm to about 200 nm, from about 200 nm to about 400 nm, from about 200 nm to about 300 nm, from about 300 nm to about 400 nm, from about 250 nm to about 350 nm, or from about 275 nm to about 325 nm). In some cases, a vesicle provided herein can have a shortest dimension (e.g., a shortest diameter) of from about from about 1 nm to about 1000 nm (e.g., from about 5 nm to about 500 nm, from about 10 nm to about 500 nm, from about 25 nm to about 500 nm, from about 50 nm to about 500 nm, from about 100 nm to about 500 nm, from about 150 nm to about 500 nm, from about 200 nm to about 500 nm, from about 250 nm to about 500 nm, from about 300 nm to about 500 nm, from about 400 nm to about 500 nm, from about 5 nm to about 450 nm, from about 5 nm to about 400 nm, from about 5 nm to about 350 nm, from about 5 nm to about 300 nm, from about 5 nm to about 250 nm, from about 5 nm to about 200 nm, from about 5 nm to about 150 nm, from about 5 nm to about 100 nm, from about 5 nm to about 50 nm, from about 5 nm to about 25 nm, from about 10 nm to about 450 nm, from about 10 nm to about 400 nm, from about 20 nm to about 400 nm, from about 50 nm to about 400 nm, from about 100 nm to about 400 nm, from about 10 nm to about 300 nm, from about 20 nm to about 300 nm, from about 50 nm to about 300 nm, from about 100 nm to about 300 nm, from about 10 nm to about 200 nm, from about 20 nm to about 200 nm, from about 50 nm to about 200 nm, from about 100 nm to about 200 nm, from about 200 nm to about 400 nm, from about 200 nm to about 300 nm, from about 300 nm to about 400 nm, from about 250 nm to about 350 nm, or from about 275 nm to about 325 nm). In some cases, a vesicle provided herein can have a longest dimension (e.g., a longest diameter) of from about 1 nm to about 1000 nm (e.g., from about 5 nm to about 500 nm, from about 10 nm to about 500 nm, from about 25 nm to about 500 nm, from about 50 nm to about 500 nm, from about 100 nm to about 500 nm, from about 150 nm to about 500 nm, from about 200 nm to about 500 nm, from about 250 nm to about 500 nm, from about 300 nm to about 500 nm, from about 400 nm to about 500 nm, from about 5 nm to about 450 nm, from about 5 nm to about 400 nm, from about 5 nm to about 350 nm, from about 5 nm to about 300 nm, from about 5 nm to about 250 nm, from about 5 nm to about 200 nm, from about 5 nm to about 150 nm, from about 5 nm to about 100 nm, from about 5 nm to about 50 nm, from about 5 nm to about 25 nm, from about 10 nm to about 450 nm, from about 10 nm to about 400 nm, from about 20 nm to about 400 nm, from about 50 nm to about 400 nm, from about 100 nm to about 400 nm, from about 10 nm to about 300 nm, from about 20 nm to about 300 nm, from about 50 nm to about 300 nm, from about 100 nm to about 300 nm, from about 10 nm to about 200 nm, from about 20 nm to about 200 nm, from about 50 nm to about 200 nm, from about 100 nm to about 200 nm, from about 200 nm to about 400 nm, from about 200 nm to about 300 nm, from about 300 nm to about 400 nm, from about 250 nm to about 350 nm, or from about 275 nm to about 325 nm) and can have a shortest dimension (e.g., a shortest diameter) of from about 1 nm to about 1000 nm (e.g., from about 5 nm to about 500 nm, from about 10 nm to about 500 nm, from about 25 nm to about 500 nm, from about 50 nm to about 500 nm, from about 100 nm to about 500 nm, from about 150 nm to about 500 nm, from about 200 nm to about 500 nm, from about 250 nm to about 500 nm, from about 300 nm to about 500 nm, from about 400 nm to about 500 nm, from about 5 nm to about 450 nm, from about 5 nm to about 400 nm, from about 5 nm to about 350 nm, from about 5 nm to about 300 nm, from about 5 nm to about 250 nm, from about 5 nm to about 200 nm, from about 5 nm to about 150 nm, from about 5 nm to about 100 nm, from about 5 nm to about 50 nm, from about 5 nm to about 25 nm, from about 10 nm to about 450 nm, from about 10 nm to about 400 nm, from about 20 nm to about 400 nm, from about 50 nm to about 400 nm, from about 100 nm to about 400 nm, from about 10 nm to about 300 nm, from about 20 nm to about 300 nm, from about 50 nm to about 300 nm, from about 100 nm to about 300 nm, from about 10 nm to about 200 nm, from about 20 nm to about 200 nm, from about 50 nm to about 200 nm, from about 100 nm to about 200 nm, from about 200 nm to about 400 nm, from about 200 nm to about 300 nm, from about 300 nm to about 400 nm, from about 250 nm to about 350 nm, or from about 275 nm to about 325 nm).

A targeting moiety located on a surface of a vesicle provided herein (e.g., a vesicle having (a) a targeting moiety located on a surface of the vesicle and (b) one or more (e.g., one, two, three, or more) inhibitors located within the vesicle) can be any appropriate type of molecule (e.g., small molecules, nucleic acid molecules, polypeptides, and combinations thereof). In some cases, a targeting moiety located on a surface of a vesicle provided herein can target (e.g., target and bind) to a molecule present on a surface of a target cell (e.g., a cancer cell). Examples of compounds that can be used as a targeting moiety located on a surface of a vesicle provided herein include, without limitation, polypeptides (e.g., antibodies) and nucleic acids (e.g., nucleic acid aptamers such as DNA aptamers and RNA aptamers).

A targeting moiety located on a surface of a vesicle provided herein can target (e.g., target and bind) any appropriate target molecule. In some cases, a target molecule can be a molecule present on a surface of a target cell (e.g., a cancer cell). For example, a target molecule can be a polypeptide present on a surface of a target cell (e.g., a cancer cell). For example, a target molecule can be a glycoprotein present on a surface of a target cell (e.g., a cancer cell). Examples of polypeptides that can be present on a surface of a target cell (e.g., a cancer cell) and can be targeted by a targeting moiety located on a surface of a vesicle provided herein include, without limitation, epithelial cellular adhesion molecule (EpCAM) molecules, EPH receptor A2 (EPHA2) polypeptides, AXL polypeptides, G Protein-coupled receptor class C group 5 member A (GPRC5a) polypeptides, GPRC5c polypeptides, epidermal growth factor receptor (EGFR) polypeptides, cytokeratin-19 (CK19) polypeptides, osteopontin (SPP1) polypeptides, and Sox9 polypeptides.

A targeting moiety located on a surface of a vesicle provided herein can target (e.g., target and bind) a molecule present on a surface of any appropriate type of cell. In some cases, a targeting moiety located on a surface of a vesicle provided herein can target (e.g., target and bind) to a molecule present on a surface of a cancer cell. Examples of cancer cells that can be targeted by a targeting moiety located on a surface of a vesicle provided herein include, without limitation, liver cancer cells, gall bladder cancer cells, small intestine cancer cells, and bile duct cancer cells (e.g., cholangiocarcinoma cancer cells).

A targeting moiety located on a surface of a vesicle provided herein can target (e.g., target and bind) a molecule present on a surface of a cell at any appropriate location within a mammal's body. Example of locations within a mammal's body where a cell can be targeted by a targeting moiety located on a surface of a vesicle provided herein include, without limitation, the liver, the gall bladder, the small intestine, and the bile duct.

Examples of targeting moieties that can be located on a surface of a vesicle provided herein include, without limitation, those set forth in Table 1.

TABLE 1
Exemplary targeting moieties.
Moiety

EpCAM aptamers
EGFR aptamers
EpCAM antibodies
EGFR antibodies
AXL aptamers
CK19 aptamers
SPP1 aptamers
Sox9 aptamers

When a targeting moiety that can be located on a surface of a vesicle provided herein is an aptamer, the aptamer can have any appropriate sequence. In some cases, an aptamer can comprise, consist essentially of, or consist of one of the nucleic acid sequences set forth in any one of SEQ ID NOs: 31-39 and 42-52. As used herein, an aptamer that “consists essentially of” the nucleic acid sequence set forth in an articulated SEQ ID NO: is an aptamer that has zero, one, or two amino acid substitutions within the articulated SEQ ID NO, that has zero, one, two, three, four, or five amino acid residues directly preceding the articulated SEQ ID NO, and/or that has zero, one, two, three, four, or five amino acid residues directly following the articulated SEQ ID NO, provided that the aptamer maintains its basic ability to target (e.g., target and bind) a molecule present on a surface of a cancer cell. In some cases, an aptamer that can be located on a surface of a vesicle provided herein can comprise, consist essentially of, or consist of one of the nucleic acid sequences set forth in Table 7 or Table 8.

In some cases, a targeting moiety that can be located on a surface of a vesicle provided herein can be as described elsewhere (see, e.g., Elkhoury et al., Pharmaceutics, 12:849 (2020) at, for example, pages 4-5).

As described herein, a vesicle provided herein can be designed to include (a) a targeting moiety located on a surface of the vesicle and (b) one or more (e.g., one, two, three, or more) inhibitors located within the vesicle. An inhibitor included in a vesicle provided herein can be any appropriate type of molecule (e.g., small molecules, nucleic acid molecules, polypeptides, and combinations thereof). An inhibitor designed to be within a vesicle provided herein can inhibit polypeptide expression or can inhibit polypeptide activity. Examples of compounds that can inhibit polypeptide expression include, without limitation, nucleic acid molecules designed to induce RNA interference of polypeptide expression (e.g., a siRNA molecule or a shRNA molecule), antisense molecules, miRNAs, and nucleic acid molecules designed to induce CRISPR interference (CRISPRi) of polypeptide expression. Examples of compounds that can inhibit polypeptide activity include, without limitation, antibodies (e.g., neutralizing antibodies) that target (e.g., target and bind) to a polypeptide, and small molecules that target (e.g., target and bind) to a polypeptide. When a compound that can inhibit polypeptide activity is a small molecule that targets (e.g., targets and binds) to a polypeptide, the small molecule can be in the form of a salt (e.g., a pharmaceutically acceptable salt).

An inhibitor included in a vesicle provided herein (e.g., a vesicle having (a) a targeting moiety located on a surface of the vesicle and (b) one or more (e.g., one, two, three, or more) inhibitors located within the vesicle) can inhibit any appropriate polypeptide. In some cases, an inhibitor included in a vesicle provided herein can inhibit a polypeptide involved in a TRAIL pathway. In some cases, an inhibitor included in a vesicle provided herein can inhibit a polypeptide involved in a PD-1/L1 pathway. In some cases, an inhibitor included in a vesicle provided herein can inhibit a polypeptide involved in cellular survival. In some cases, an inhibitor included in a vesicle provided herein can inhibit a polypeptide involved in pro-cancer processes. Examples of polypeptides that can be inhibited by an inhibitor included in a vesicle provided herein include, without limitation, Src family kinases (e.g., LCK polypeptides), TRAIL polypeptides, YAP polypeptides, TAZ polypeptides, EGFR polypeptides, and AXL polypeptides.

In some cases, a vesicle provided herein can be designed to include (a) a targeting moiety located on a surface of the vesicle and (b) one or more (e.g., one, two, three, or more) inhibitors of a LCK polypeptide (e.g., one or more inhibitors of LCK polypeptide expression, one or more inhibitors of LCK polypeptide activity, or a combination thereof). An inhibitor of a LCK polypeptide can inhibit any appropriate LCK polypeptide. Examples of LCK polypeptides that can be inhibited by an inhibitor included in a vesicle provided herein include, without limitation, those set forth in the NCBI databases at, for example, accession no. P06239, accession no. D3DPP8, accession no. P07100, accession no. Q12850, accession no. Q13152, and accession no. Q5TDH8. In some cases, nucleic acid molecules designed to induce RNA interference of LCK polypeptide expression can be designed based on the nucleotide sequence of nucleic acid that encodes a LCK polypeptide (e.g., a LCK gene sequence). Examples of nucleotide sequences that encode a LCK polypeptide and can be used to design a nucleic acid molecule designed to induce RNA interference of LCK polypeptide expression include, without limitation, those set forth in the NCBI databases at, for example, accession no. NG_023387 (version NG_023387.1), accession no. NM_001042771, accession no. NM_001330468, accession no. NM_005356, accession no. XM_011541453, accession no. NM_001162432, accession no. NM_001162433, accession no. NM_010693, accession no. and accession no. XM_006502818. Examples of inhibitors of a LCK polypeptide that can be included in a vesicle provided herein include, without limitation, those set forth in Table 2.

TABLE 2
Exemplary LCK inhibitors.

| LCK inhibitor | |
|---|---|
| siLCK | horizon ™ cat# L-003151-00-0005 |
| siLCK | horizon ™ cat# L-043878-00-0005 |
| Dasatinib | Selleckchem cat#S5254 |
| Saracatinib | Selleckchem cat#S1006 |

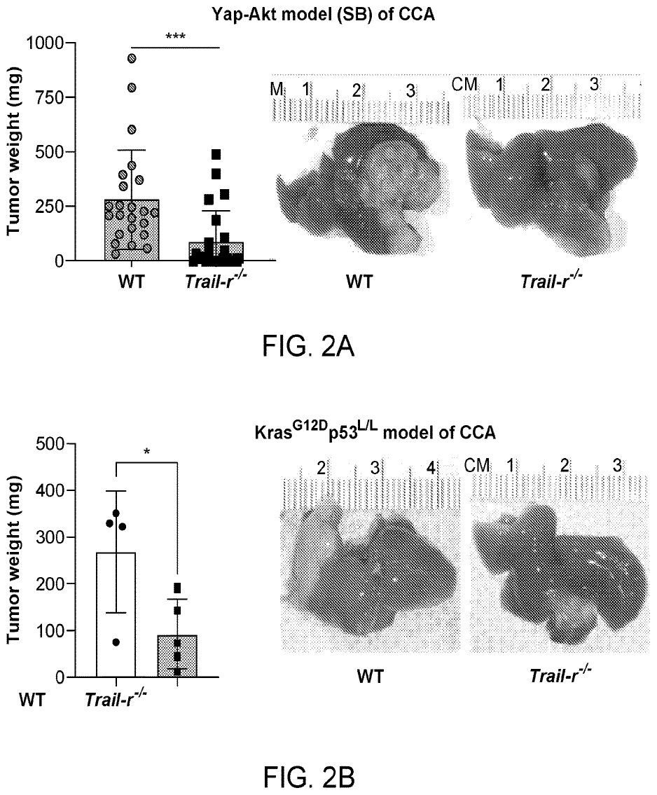

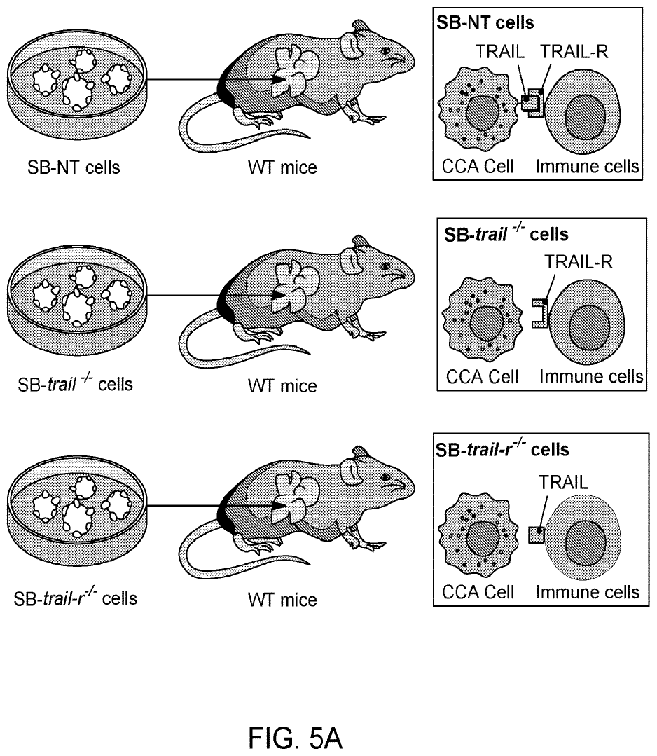

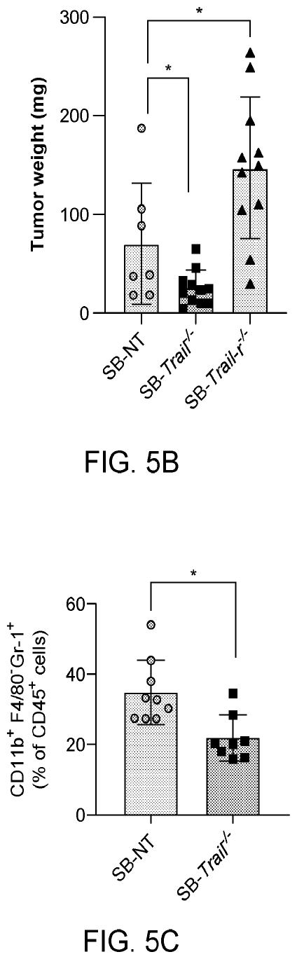

In some cases, an inhibitor of a LCK polypeptide that can be included in a vesicle provided herein can be as described elsewhere (see, e.g., Sugihara et al., Mol. Cancer Res., 16 (10): 1556-1567 (2018) at, for example, FIG. 2 and FIG. 5).

In some cases, a vesicle provided herein can be designed to include (a) a targeting moiety located on a surface of the vesicle and (b) one or more (e.g., one, two, three, or more) inhibitors of a LCK polypeptide (e.g., one or more inhibitors of LCK polypeptide expression, one or more inhibitors of LCK polypeptide activity, or a combination thereof) where the only inhibitors of a polypeptide's expression or activity within the vesicle are those that inhibit a LCK polypeptide. In some cases, a vesicle provided herein can be designed to include (a) a targeting moiety located on a surface of the vesicle and (b) one or more (e.g., one, two, three, or more) inhibitors of a LCK polypeptide (e.g., one or more inhibitors of LCK polypeptide expression, one or more inhibitors of LCK polypeptide activity, or a combination thereof) in combination with one or more other inhibitors (e.g., inhibitors of a TRAIL polypeptide, inhibitors of a YAP polypeptide, inhibitors of a TAZ polypeptide, or combinations thereof).

In some cases, a vesicle provided herein can be designed to include (a) a targeting moiety located on a surface of the vesicle and (b) one or more (e.g., one, two, three, or more) inhibitors of a TRAIL polypeptide (e.g., one or more inhibitors of TRAIL polypeptide expression, one or more inhibitors of TRAIL polypeptide activity, or a combination thereof). An inhibitor of a TRAIL polypeptide can inhibit any appropriate TRAIL polypeptide. Examples of TRAIL polypeptides that can be inhibited by an inhibitor included in a vesicle provided herein include, without limitation, those set forth in the NCBI databases at, for example, accession no. P50591 (version P50591.1). In some cases, nucleic acid molecules designed to induce RNA interference of TRAIL polypeptide expression can be designed based on the nucleotide sequence of nucleic acid that encodes a TRAIL polypeptide (e.g., a TNFSF10 gene sequence). Examples of nucleotide sequences that encode a TRAIL polypeptide and can be used to design nucleic acid molecules designed to induce RNA interference of TRAIL polypeptide expression include, without limitation, those set forth in the NCBI databases at, for example, accession no. NM_001190942 (version NM 001190942.2, accession no. NM_003810 (version NM_003810.4), accession no. NM_001190943 (version NM_001190943.2), and accession no. NM_009425. Examples of inhibitors of a TRAIL polypeptide that can be included in a vesicle provided herein include, without limitation, those set forth in Table 3.

TABLE 3
Exemplary TRAIL inhibitors.

| TRAIL inhibitor | |
|---|---|
| siTRAIL | horizon ™ cat# L-047370-00-0005 |

In some cases, a vesicle provided herein can be designed to include (a) a targeting moiety located on a surface of the vesicle and (b) one or more (e.g., one, two, three, or more) inhibitors of a TRAIL polypeptide (e.g., one or more inhibitors of TRAIL polypeptide expression, one or more inhibitors of TRAIL polypeptide activity, or a combination thereof) where the only inhibitors of a polypeptide's expression or activity within the vesicle are those that inhibit a TRAIL polypeptide. In some cases, a vesicle provided herein can be designed to include (a) a targeting moiety located on a surface of the vesicle and (b) one or more (e.g., one, two, three, or more) inhibitors of a TRAIL polypeptide (e.g., one or more inhibitors of TRAIL polypeptide expression, one or more inhibitors of TRAIL polypeptide activity, or a combination thereof) in combination with one or more other inhibitors (e.g., inhibitors of a LCK polypeptide, inhibitors of a YAP polypeptide, inhibitors of a TAZ polypeptide, or combinations thereof).

In some cases, a vesicle provided herein can be designed to include (a) a targeting moiety located on a surface of the vesicle and (b) one or more (e.g., one, two, three, or more) inhibitors of a YAP polypeptide (e.g., one or more inhibitors of YAP polypeptide expression, one or more inhibitors of YAP polypeptide activity, or a combination thereof). An inhibitor of a YAP polypeptide can inhibit any appropriate YAP polypeptide. Examples of YAP polypeptides that can be inhibited by an inhibitor included in a vesicle provided herein include, without limitation, those set forth in the National Center for Biotechnology Information (NCBI) databases at, for example, accession no. XP_005271435 (version XP_005271435.1), accession no. XP_005271438 (version XP_005271438.1), accession no. XP 005271437 (version XP_005271437.1), accession no. XP_005271440 (version XP_005271440.1), accession no. NP_001269030 (version NP_001269030.1), accession no. NP_001269029 (version NP_001269029.1), accession no. NP_001123617 (version NP_001123617.1), accession no. NP_001181973 (version NP_001181973.1), accession no. NP_001269028 (version NP_001269028.1), accession no. NP_001269027 (version NP_001269027.1), accession no. NP_001269026 (version NP_001269026.1), accession no. NP_006097 (version NP_006097.2), accession no. XP_011540857 (version XP_011540857.1), and accession no. NP_001181974 (version NP_001181974.1). In some cases, nucleic acid molecules designed to induce RNA interference of YAP polypeptide expression can be designed based on the nucleotide sequence of nucleic acid that encodes a YAP polypeptide (e.g., a YAP1 gene sequence). Examples of nucleotide sequences that encode a YAP polypeptide and can be used to design nucleic acid molecules designed to induce RNA interference of YAP polypeptide expression include, without limitation, those set forth in the NCBI databases at, for example, accession no. NG_029530 (version NG_029530.2), accession no. XM_005271378 (version XM_005271378.4), accession no. XM_005271381 (version XM_005271381.4), accession no. XM_005271380 (version XM_005271380.4), accession no. XM_005271383 (version XM_005271383.4), accession no. NM_001282101 (version NM_001282101.2), accession no. NM_001282100 (version NM_001282100.2), accession no. NM_001130145 (version NM_001130145.3), accession no. NM_001195044 (version NM_001195044.2), accession no. NM_001282099 (version NM_001282099.2), accession no. NM_001282098 (version NM_001282098.2), accession no. NM_001282097 (version NM_001282097.2), accession no. NM_006106 (version NM_006106.5), accession no. XM_011542555 (version XM_011542555.3), and accession no. NM_001195045 (version NM_001195045.2). Examples of inhibitors of a YAP polypeptide that can be included in a vesicle provided herein include, without limitation, those set forth in Table 4.

TABLE 4
Exemplary YAP inhibitors.

| YAP inhibitor | |
|---|---|
| siYAP | horizon ™ cat# L-012200-00-0005 |
| siYAP | horizon ™ cat# L-046247-01-0005 |
| CA3 | Selleckchem cat#S8661 |
| Verteporfin | Selleckchem cat#S1786 |

In some cases, an inhibitor of a YAP polypeptide that can be included in a vesicle provided herein can be as described elsewhere (see, e.g., Han et al., Cancers (Basel), 14 (11): 2733 (2022) at, for example, FIG. 8 and Table 2; and Won et al., Nat. Commun., 13 (1): 3117 (2022) at, for example, Figure S9).

In some cases, a vesicle provided herein can be designed to include (a) a targeting moiety located on a surface of the vesicle and (b) one or more (e.g., one, two, three, or more) inhibitors of a YAP polypeptide (e.g., one or more inhibitors of YAP polypeptide expression, one or more inhibitors of YAP polypeptide activity, or a combination thereof) where the only inhibitors of a polypeptide's expression or activity within the vesicle are those that inhibit a YAP polypeptide. In some cases, a vesicle provided herein can be designed to include (a) a targeting moiety located on a surface of the vesicle and (b) one or more (e.g., one, two, three, or more) inhibitors of a YAP polypeptide (e.g., one or more inhibitors of YAP polypeptide expression, one or more inhibitors of YAP polypeptide activity, or a combination thereof) in combination with one or more other inhibitors (e.g., inhibitors of a TRAIL polypeptide, inhibitors of a LCK polypeptide, inhibitors of a TAZ polypeptide, or combinations thereof).

In some cases, a vesicle provided herein can be designed to include (a) a targeting moiety located on a surface of the vesicle and (b) one or more (e.g., one, two, three, or more) inhibitors of a TAZ polypeptide (e.g., one or more inhibitors of TAZ polypeptide expression, one or more inhibitors of TAZ polypeptide activity, or a combination thereof). An inhibitor of a TAZ polypeptide can inhibit any appropriate TAZ polypeptide. Examples of TAZ polypeptides that can be inhibited by an inhibitor included in a vesicle provided herein include, without limitation, those set forth in the NCBI databases at, for example, accession no. NP_001335291 (version NP_001335291.1), accession no. XP_047303889 (version XP_047303889.1), accession no. XP_047303887 (version XP_047303887.1), accession no. XP_047303888 (version XP_047303888.1), accession no. NP_001161750 (version NP_001161750.1), accession no. XP_016861611 (version XP_016861611.1), accession no. XP 047303886 (version XP_047303886.1), accession no. NP_001161752 (version NP_001161752.1), and accession no. NP_056287 (version NP_056287.1). In some cases, nucleic acid molecules designed to induce RNA interference of TAZ polypeptide expression can be designed based on the nucleotide sequence of nucleic acid that encodes a TAZ polypeptide (e.g., a WWTR1 gene sequence). Examples of nucleotide sequences that encode a TAZ polypeptide and can be used to design nucleic acid molecules designed to induce RNA interference of TAZ polypeptide expression include, without limitation, those set forth in the NCBI databases at, for example, accession no. NM_001348362 (version NM_001348362.2), accession no. XM_047447933 (version XM_047447933.1), accession no. XM 047447931 (version XM_047447931.1), accession no. XM_047447932 (version XM_047447932.1), accession no. NM_001168278 (version NM_001168278.3), accession no. XM_017006122 (version XM_017006122.2), accession no. XM_047447930 (version XM_047447930.1), accession no. NM_001168280 (version NM_001168280.3), and accession no. NM 015472 (version NM_015472.6). Examples of inhibitors of a TAZ polypeptide that can be included in a vesicle provided herein include, without limitation, those set forth in Table 5.

TABLE 5
Exemplary TAZ inhibitors.

| TAZ inhibitor | |
|---|---|
| siTAZ | horizon ™ cat# L-058248-01-0005 |
| siTAZ | Sigma-Aldrich cat # WD09662428 |

In some cases, a vesicle provided herein can be designed to include (a) a targeting moiety located on a surface of the vesicle and (b) one or more (e.g., one, two, three, or more) inhibitors of a TAZ polypeptide (e.g., one or more inhibitors of TAZ polypeptide expression, one or more inhibitors of TAZ polypeptide activity, or a combination thereof) where the only inhibitors of a polypeptide's expression or activity within the vesicle are those that inhibit a TAZ polypeptide. In some cases, a vesicle provided herein can be designed to include (a) a targeting moiety located on a surface of the vesicle and (b) one or more (e.g., one, two, three, or more) inhibitors of a TAZ polypeptide (e.g., one or more inhibitors of TAZ polypeptide expression, one or more inhibitors of TAZ polypeptide activity, or a combination thereof) in combination with one or more other inhibitors (e.g., inhibitors of a TRAIL polypeptide, inhibitors of a LCK polypeptide, inhibitors of a YAP polypeptide, or combinations thereof).

In some cases, a vesicle provided herein (e.g., a vesicle having (a) a targeting moiety located on a surface of the vesicle and (b) one or more (e.g., one, two, three, or more) inhibitors located within the vesicle) also can include one or more (e.g., one, two, three, or more) additional compounds. For example, a vesicle provided herein can include one or more imaging agents located within the vesicle and/or on the surface of the vesicle. For example, a vesicle provided herein can be designed to include one or more imaging agents located on a surface of the vesicle. Examples of imaging agents that be located within a vesicle and/or on the surface of the vesicle provided herein include, without limitation, fluorophores, fluorescent dyes, and radionucleotides.

In some cases, a vesicle provided herein can include a first inhibitor located within the vesicle and a second inhibitor located within the vesicle, and can include a targeting moiety located on a surface of the vesicle. For example, a vesicle provided herein can include a first inhibitor that can inhibit expression or activity of a LCK polypeptide and a second inhibitor that can inhibit expression or activity of a TRAIL polypeptide located within the vesicle, and can include an aptamer located on a surface of the vesicle that can bind to a molecule present on the surface of a cancer cell.

In some cases, a vesicle provided herein can include a first inhibitor located within the vesicle, a second inhibitor located within the vesicle, a third inhibitor located within the vesicle, and can include a targeting moiety located on a surface of the vesicle. For example, a vesicle provided herein can include a first inhibitor that can inhibit expression or activity of a YAP polypeptide, a second inhibitor that can inhibit expression or activity of a LCK polypeptide, a third inhibitor that can inhibit expression or activity of a TRAIL polypeptide located within the vesicle, and can include an aptamer located on a surface of the vesicle that can bind to a molecule present on the surface of a cancer cell.

In some cases, a vesicle provided herein can include a first inhibitor located within the vesicle, a second inhibitor located within the vesicle, a third inhibitor located within the vesicle, and can include a targeting moiety located on a surface of the vesicle. For example, a vesicle provided herein can include a first inhibitor that can inhibit expression or activity of a TAZ polypeptide, a second inhibitor that can inhibit expression or activity of a LCK polypeptide, a third inhibitor that can inhibit expression or activity of a TRAIL polypeptide located within the vesicle, and can include an aptamer located on a surface of the vesicle that can bind to a molecule present on the surface of a cancer cell.

A vesicle provided herein (e.g., a vesicle having (a) a targeting moiety located on a surface of the vesicle and (b) one or more (e.g., one, two, three, or more) inhibitors located within the vesicle) can be made using any appropriate method. For example, filtration, extrusion, differential centrifugation, high shear homogenization, hot homogenization, cold homogenization, ultrasonication/high speed homogenization, probe ultrasonication, bath ultrasonication, solvent emulsification/evaporation, micro emulsion based solid lipid nanoparticles (SLN) preparations, SLN preparation by using supercritical fluid, spray drying methods, and double emulsion methods can be used to make a vesicle provided herein. In some cases, a vesicle provided herein (e.g., a vesicle having (a) a targeting moiety located on a surface of the vesicle and (b) one or more (e.g., one, two, three, or more) inhibitors located within the vesicle) can be made as described in Example 1 or Example 2. In some cases, a vesicle provided herein (e.g., a vesicle having (a) a targeting moiety located on a surface of the vesicle and (b) one or more (e.g., one, two, three, or more) inhibitors located within the vesicle) can be made as described elsewhere (see, e.g., Mukherjee et al., Indian J. Pharm. Sci., 71 (4): 349-58 (2009) at, for example, Table 3).

This document also provides compositions containing one or more vesicles provided herein (e.g., one or more vesicles each including (a) a targeting moiety located on a surface of the vesicle and (b) one or more (e.g., one, two, three, or more) inhibitors located within the vesicle). In some cases, one or more (e.g., one, two, three, four, or more) vesicles provided herein can be formulated into a composition (e.g., a pharmaceutically acceptable composition) for administration to a mammal (e.g., a human) having cancer (e.g., cholangiocarcinoma). For example, one or more vesicles provided herein can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline such as phosphate buffered saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, a composition including one or more vesicles provided herein (e.g., one or more vesicles each including (a) a targeting moiety located on a surface of the vesicle and (b) one or more (e.g., one, two, three, or more) inhibitors located within the vesicle) can include a plurality of identical vesicles.

In some cases, a composition including one or more vesicles provided herein (e.g., one or more vesicles each including (a) a targeting moiety located on a surface of the vesicle and (b) one or more (e.g., one, two, three, or more) inhibitors located within the vesicle) can include a population of two or more (e.g., two, three, four, five, or more) different vesicles. For example, a composition can be designed to include two populations of vesicles, with the first population containing vesicles having one or more inhibitors of a LCK polypeptide and the second population containing vesicles having one or more inhibitors of a TRAIL polypeptide. In another example, a composition can be designed to include three populations of vesicles, with the first population containing vesicles having one or more inhibitors of a YAP polypeptide, the second population containing vesicles having one or more inhibitors of a LCK polypeptide, and the third population containing vesicles having one or more inhibitors of a TRAIL polypeptide. In another example, a composition can be designed to include three populations of vesicles, with the first population containing vesicles having one or more inhibitors of a TAZ polypeptide, the second population containing vesicles having one or more inhibitors of a LCK polypeptide, and the third population containing vesicles having one or more inhibitors of a TRAIL polypeptide.

In some cases, when a composition containing one or more vesicles provided herein (e.g., one or more vesicles each including (a) a targeting moiety located on a surface of the vesicle and (b) one or more (e.g., one, two, three, or more) inhibitors located within the vesicle) is administered to a mammal (e.g., a human) having cancer (e.g., cholangiocarcinoma), the composition can be designed for oral or parenteral (including, without limitation, subcutaneous, intramuscular, intravenous, intradermal, intra-cerebral, intrathecal, intraabdominal, and intraperitoneal injections) administration to the mammal. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

In some cases, a composition containing one or more vesicles provided herein (e.g., one or more vesicles each including (a) a targeting moiety located on a surface of the vesicle and (b) one or more (e.g., one, two, three, or more) inhibitors located within the vesicle) can be in the form of a sterile injectable suspension (e.g., a sterile injectable aqueous or oleaginous suspension). This suspension may be formulated using, for example, suitable dispersing or wetting agents (such as, for example, Tween 80) and/or suspending agents. The sterile injectable preparation can be a sterile injectable solution or suspension in a non-toxic parenterally-acceptable diluent or solvent, for example, as a solution in 1,3-butanediol. Examples of acceptable vehicles and solvents that can be used include, without limitation, saline, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides.

In some cases, a composition containing one or more vesicles provided herein (e.g., one or more vesicles each including (a) a targeting moiety located on a surface of the vesicle and (b) one or more (e.g., one, two, three, or more) inhibitors located within the vesicle) can be presented in unit-dose or multi-dose containers, for example, sealed ampules and vials, and may be stored in a freeze dried (lyophilized) condition requiring only the addition of the sterile liquid carrier, for example water for injections, immediately prior to use.

This document also provides methods and materials for using one or more vesicles provided herein (e.g., a composition including one or more vesicles that include (a) a targeting moiety located on a surface of the vesicle and (b) one or more (e.g., one, two, three, or more) inhibitors). For example, one or more vesicles provided herein (e.g., a composition including one or more vesicles that include (a) a targeting moiety located on a surface of the vesicle and (b) one or more (e.g., one, two, three, or more) inhibitors located within the vesicle) can be administered to a mammal having cancer (e.g., cholangiocarcinoma) to treat the mammal's cancer.

In some cases, using one or more vesicles provided herein (e.g., a composition including one or more vesicles that include (a) a targeting moiety located on a surface of the vesicle and (b) one or more (e.g., one, two, three, or more) inhibitors located within the vesicle) can be effective to reduce the size of the cancer in the mammal. For example, one or more vesicles provided herein can be administered to a mammal (e.g., a human) in need thereof (e.g., a human having cancer) as described herein to reduce the size of the cancer in the mammal. In some cases, the methods and materials provided herein can be used as described herein to reduce the number of cancer cells in the mammal by, for example, 10, 20, 30, 40, 50, 60, 70, 80, 90, 95, or more percent. In some cases, the methods and materials provided herein can be used as described herein to reduce the volume of one or more tumors in the mammal by, for example, 10, 20, 30, 40, 50, 60, 70, 80, 90, 95, or more percent.

In some cases, using one or more vesicles provided herein (e.g., a composition including one or more vesicles that include (a) a targeting moiety located on a surface of the vesicle and (b) one or more (e.g., one, two, three, or more) inhibitors located within the vesicle) can be effective to improve survival of the mammal. For example, one or more vesicles provided herein can be administered to a mammal (e.g., a human) in need thereof (e.g., a human having cancer) as described herein to improve survival of the mammal. In some cases, the methods and materials provided herein can be used as described herein to improve the survival of the mammal by, for example, 10, 20, 30, 40, 50, 60, 70, 80, 90, 95, or more percent. In some cases, the methods and materials provided herein can be used as described herein to improve the survival of the mammal by, for example, at least 6 months (e.g., about 6 months, about 8 months, about 10 months, about 1 year, about 1.5 years, about 2 years, about 2.5 years, about 3 years, about 4 years, about 5 years, or more).

Any appropriate mammal having cancer (e.g., cholangiocarcinoma) can be treated as described herein (e.g., by administering one or more vesicles that include (a) a targeting moiety located on a surface of the vesicle and (b) one or more (e.g., one, two, three, or more) inhibitors located within the vesicle). Examples of mammals that can have cancer and can be treated as described herein include, without limitation, humans, non-human primates (e.g., monkeys), horses, bovine species, porcine species, dogs, cats, mice, and rats. In some cases, a human having cancer can be treated by using one or more vesicles that include (a) a targeting moiety located on a surface of the vesicle and (b) one or more (e.g., one, two, three, or more) inhibitors located within the vesicle.

A mammal (e.g., a human) having any type of cancer can be treated as described herein (e.g., by using one or more vesicles each including (a) one or more (e.g., one, two, three, or more) inhibitors located within the vesicle and (b) a targeting moiety located on a surface of the vesicle). A cancer can be a primary cancer or a metastatic cancer. In some cases, a cancer that can be treated as described herein can include one or more solid tumors. In some cases, a cancer that can be treated as described herein can be a blood cancer. In some cases, a cancer treated as described herein can be resistant to one or more immunotherapies. Examples of cancers that can be treated as described herein include, without limitation, cholangiocarcinomas, liver cancers, gall bladder cancers, gastrointestinal cancers (e.g., small intestine cancers), colon cancers, rectal cancers, lung cancers, breast cancers, esophageal cancers, ovarian cancers, and renal cancers.

In some cases, the methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having cancer, a mammal can treated as described herein (e.g., by administering one or more vesicles that include (a) a targeting moiety located on a surface of the vesicle and (b) one or more (e.g., one, two, three, or more) inhibitors located within the vesicle).

One or more vesicles provided herein (e.g., a composition including one or more vesicles that include (a) a targeting moiety located on a surface of the vesicle and (b) one or more (e.g., one, two, three, or more) inhibitors located within the vesicle) can be administered to a mammal (e.g., a human) by any appropriate route (e.g., intratumoral injection and intravenous injection). In some cases, one or more vesicles provided herein can be administered to a mammal (e.g., a human) by injection into the mammal's blood stream. In some cases, one or more vesicles provided herein can be administered to a mammal (e.g., a human) by intravenous injection. In some cases, one or more vesicles provided herein can be administered to a mammal (e.g., a human) by intratumoral injection.

A composition containing one or more vesicles provided herein (e.g., a composition including one or more vesicles that include (a) a targeting moiety located on a surface of the vesicle and (b) one or more (e.g., one, two, three, or more) inhibitors located within the vesicle) can be administered to a mammal (e.g., a human) having cancer (e.g., cholangiocarcinoma) in any appropriate amount (e.g., any appropriate dose). An effective amount of a composition containing one or more vesicles provided herein can be any amount that can treat a mammal having cancer (e.g., cholangiocarcinoma) as described herein without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer in the mammal being treated may require an increase or decrease in the actual effective amount administered.

A composition containing one or more vesicles provided herein (e.g., a composition including one or more vesicles that include (a) a targeting moiety located on a surface of the vesicle and (b) one or more (e.g., one, two, three, or more) inhibitors located within the vesicle) can be administered to a mammal (e.g., a human) having cancer (e.g., cholangiocarcinoma) in any appropriate frequency. The frequency of administration can be any frequency that can treat a mammal having cancer (e.g., cholangiocarcinoma) without producing significant toxicity to the mammal. For example, the frequency of administration can be from about twice a day to about one every other day, once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more vesicles provided herein (e.g., a composition including one or more vesicles that include (a) a targeting moiety located on a surface of the vesicle and (b) one or more (e.g., one, two, three, or more) inhibitors located within the vesicle) can be administered to a mammal (e.g., a human) having cancer (e.g., cholangiocarcinoma) for any appropriate duration. An effective duration for administering or using one or more vesicles provided herein can be any duration that can treat a mammal having cancer (e.g., cholangiocarcinoma) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, the methods for treating a mammal (e.g., a human) having cancer (e.g., cholangiocarcinoma) described herein can include using one or more vesicles provided herein (e.g., a composition including one or more vesicles that include (a) a targeting moiety located on a surface of the vesicle and (b) one or more (e.g., one, two, three, or more) inhibitors located within the vesicle) as the sole active agent to treat the mammal. For example, a composition containing one or more vesicles that include (a) a targeting moiety located on a surface of the vesicle and (b) one or more (e.g., one, two, three, or more) inhibitors located within the vesicle can used as the sole active ingredient to treat a mammal having cancer (e.g., cholangiocarcinoma).

In some cases, the methods for treating a mammal (e.g., a human) having cancer (e.g., cholangiocarcinoma) described herein can include administering to the mammal one or more (e.g., one, two, three, four, five or more) additional agents used to treat cancer to the mammal and/or can include performing one or more additional therapies used to treat cancer on the mammal. For example, a combination therapy used to treat cancer (e.g., cholangiocarcinoma) can include administering one or more vesicles provided herein (e.g., a composition including one or more vesicles that include (a) a targeting moiety located on a surface of the vesicle and (b) one or more (e.g., one, two, three, or more) inhibitors located within the vesicle), and also administering one or more (e.g., one, two, three, four, five or more) additional agents used to treat cancer (e.g., cholangiocarcinoma). In some cases, an additional agent that can be administered to a mammal to treat cancer (e.g., cholangiocarcinoma) can be a chemotherapeutic agent. In some cases, an agent that can be administered to a mammal to treat cancer (e.g., cholangiocarcinoma) can be an immune checkpoint inhibitors (e.g., one or more PD1 inhibitors, one or more PD-L1 inhibitors, one or more CTLA4 inhibitors, or combinations thereof). In some cases, an additional agent that can be administered to a mammal to treat cancer (e.g., cholangiocarcinoma) can be a cytotoxic agent. Examples of additional agents that can be administered to a mammal to treat cancer (e.g., cholangiocarcinoma) include, without limitation, ipilimumab, nivolumab, pembrolizumab, atezolizumab, avelumab, durvalumab, cemiplimab, gemcitabine, 5-fluorouracil (5-FU), oxaliplatin, paclitaxel (e.g., albumin-bound paclitaxel), capecitabine, cisplatin, irinotecan, pemigatinib, infigratinib, futibatinib, ivosidenib, and any combination thereof. In cases where one or more vesicles provided herein (e.g., a composition including one or more vesicles that include (a) a targeting moiety located on a surface of the vesicle and (b) one or more (e.g., one, two, three, or more) inhibitors located within the vesicle) are used in combination with one or more additional agents used to treat cancer (e.g., cholangiocarcinoma), the one or more additional agents can be administered at the same time (e.g., in a single composition containing both one or more vesicles provided herein and the one or more additional agents) or independently. For example, the one or more vesicles provided herein can be administered first, and the one or more additional agents administered second, or vice versa.

In some cases, a combination therapy used to treat cancer (e.g., cholangiocarcinoma) can include using one or more vesicles provided herein (e.g., a composition including one or more vesicles that include (a) a targeting moiety located on a surface of the vesicle and (b) one or more (e.g., one, two, three, or more) inhibitors located within the vesicle), and also can include performing one or more (e.g., one, two, three, four, five or more) additional therapies used to treat cancer (e.g., cholangiocarcinoma) on the mammal. Examples of additional therapies used to treat cancer (e.g., cholangiocarcinoma) include, without limitation, surgery (e.g., transplantation), radiation therapies, adoptive cell transfer therapies, and/or embolization (e.g., radioembolization, bland embolization, and chemoembolization). In cases where one or more vesicles provided herein (e.g., a composition including one or more vesicles that include (a) a targeting moiety located on a surface of the vesicle and (b) one or more (e.g., one, two, three, or more) inhibitors located within the vesicle) are used in combination with one or more additional therapies used to treat cancer, the one or more additional therapies can be performed at the same time or independently of administering the one or more vesicles provided herein. For example, administering one or more vesicles provided herein can be performed before, during, or after the one or more additional therapies are performed.

In some cases, the size of the cancer (e.g., the number of cancer cells and/or the volume of one or more tumors) present within a mammal and/or the severity of one or more symptoms of the cancer being treated can be monitored. Any appropriate method can be used to determine whether or not the size of the cancer present within a mammal is reduced. For example, imaging techniques can be used to assess the size of the cancer present within a mammal (e.g., a human).

The invention will be further described in the following examples, which do not limit the scope of the invention described in the claims.

### EXAMPLES

### Example 1: Nanoparticles Targeting TRAIL⁺ Cancer Cells For Cancer Immunotherapeutics

This Example describes using nanovesicles for siRNA-directed deletion of TRAIL expression in CCA cells.

### TRAIL Signaling and the Tumor Immune Microenvironment (TIME)

Immunocompetent murine models of primary liver cancer were developed. Briefly, murine tumor cells (SB cells) were isolated from a genetic mouse model of CCA driven by the oncogenes Akt and yes-associated protein (YAP^{S127A}). Implantation of SB cells into livers of wild-type (WT) C57BL/6J mice resulted in a unique syngeneic, orthotopic model of CCA. A second syngeneic model was also established using murine cells isolated from KrasG12D p53L/L murine CCA tumors. These models were used to assess the influence of TRAIL-TRAIL-R on the TIME.

Single cell RNA sequencing (scRNA seq) analysis of freshly resected human CCA tumors (n=10) was done. It was demonstrated that CCA cells express TRAIL and MDSCs express TRAIL-R 1-4 in human CCA (FIG. 1). Accordingly, the TRAIL-TRAIL-R system may facilitate CCA progression.

To examine the role of the TRAIL-TRAIL-R system in the CCA TIME, murine CCA cells that express TRAIL were implanted into livers of C57BL/6J and Trail-r^{-/-} mice. Hence, in this model the host immune cells express TRAIL but not the receptor; and are capable of inducing TRAIL-mediated apoptosis in CCA cells but are resistant to potential TRAIL-mediated immunosuppression. Implantation of murine CCA cells (SB or KrasG12Dp53L/L) into Trail-r^{-/-} mice resulted in a significant reduction in tumor volumes compared to WT mice (FIG. 2A and FIG. 2B). These results suggest that TRAIL-R⁺ host immune cells facilitate tumor progression.

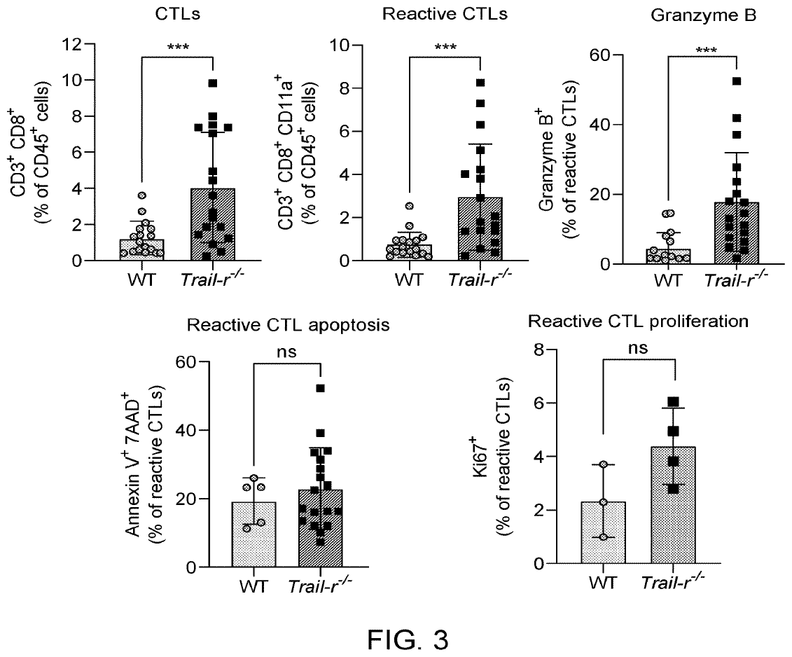

Tumors arising in Trail-r^{-/-} mice exhibited enhanced CD8⁺ T cell infiltration and tumor-reactive CD8⁺ T cells compared to WT mice tumors (FIG. 3). Reactive CTLs express CD11a, an integrin, which is upregulated in effector and memory CD8⁺ T cells. Trail-r^{-/-} tumors also had enhanced CTL effector function (granzyme B) (FIG. 3). However, no increase in CTL apoptosis or proliferation in Trail-r^{-/-} tumors (FIG. 3) was observed. There was no difference in the abundance of TAMs, dendritic cells, NK cells, or CD4⁺ T cells.

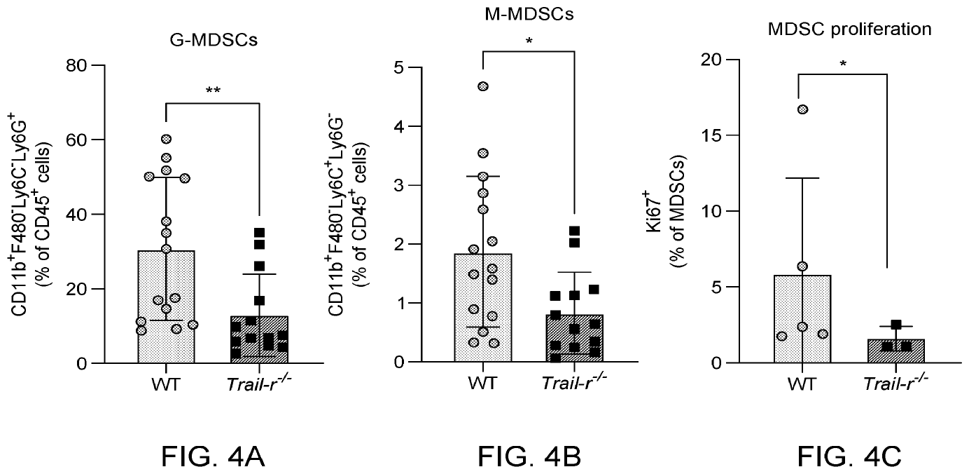

Trail-r^{-/-} tumors exhibited a significant reduction in MDSCs, both CD11b⁺Ly6G Ly6C^{low} G-MDSCs and CD11b⁺Ly6G^{low}Ly6C⁺ M-MDSCs (FIG. 4A and FIG. 4B). Moreover, Ki67 staining demonstrated that MDSCs in Trail-r^{-/-} tumors had enhanced proliferation compared to MDSCs in WT mouse tumors (FIG. 4C). No change in apoptosis was noted in the MDSCs in Trail-r^{-/-} tumors, suggesting that these cells are resistant to TRAIL-mediated apoptosis. Overall, these results suggest that TRAIL signaling augments MDSC abundance in tumors via enhanced proliferation.

To ascertain whether these findings were related to the influence of TRAIL-TRAIL-R on the immune microenvironment rather than an intrinsic change in the immune response causes by genetic deletion of Trail-r, SB cells with CRISPR-Cas9-mediated knockdown of Trail (SB-Trail^{-/-}) and mouse TRAIL-R (SB-Trail-r^{-/-}) were generated (FIG. 5A). Implantation of SB-Trail^{-/-} into WT mice resulted in a significant reduction in the tumor burden compared to control SB cell (non-targeting (NT)) implantation (FIG. 5B). SB-Trail-r^{-/-} cell implantation into WT mice resulted in larger tumors compared to control (FIG. 5B). There was a significant decrease in MDSCs in the SB-Trail^{-/-} tumors (FIG. 5C). These results suggest that cancer cell restricted deletion of Trail hinders tumor growth as Trail⁺ cancer cells cannot trigger a TRAIL-R mediated increase in MDSC abundance.

Together, these results demonstrate that TRAIL signaling has a direct effect on the immune microenvironment, independent of tumor cells.

### TRAIL-TRAIL-R and Tumor Immune Evasion

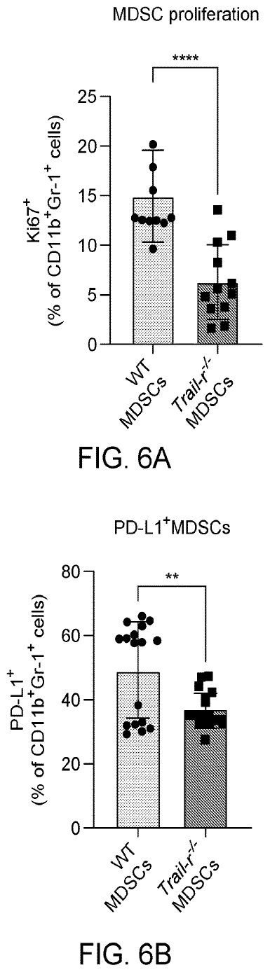

BM-derived MDSCs were cocultured with murine tumor cells (SB cells) and MDSCs were subsequently harvested for further analysis via flow cytometry. WT MDSCs had a significant increase in proliferation when co-cultured with SB cells. Furthermore, MDSCs from Trail-r^{-/-} (Trail-r^{-/-} MDSC) had a significant decrease in proliferation compared to MDSCs from WT mice (WT MDSC) when cocultured with tumor cells (FIG. 6A), suggesting that TRAIL⁺ cancer cells promote an increase in MDSC abundance by enhancing MDSC proliferation. This experimental design was repeated using T cells rather than MDSCs, and no direct effect of TRAIL signaling on T cells was observed. It was also assessed whether Trail-r deficiency affects the immunosuppressive function of MDSCs. When cocultured with Trail⁺SB cells, Trail-r^{-/-} MDSCs had decreased expression of the immune checkpoint molecule PD-L1 (FIG. 6B), suggesting that TRAIL signaling enhances the immunosuppressive function of MDSCs.

### Non-Canonical TRAIL Signaling Augments MDSC Abundance and Proliferation

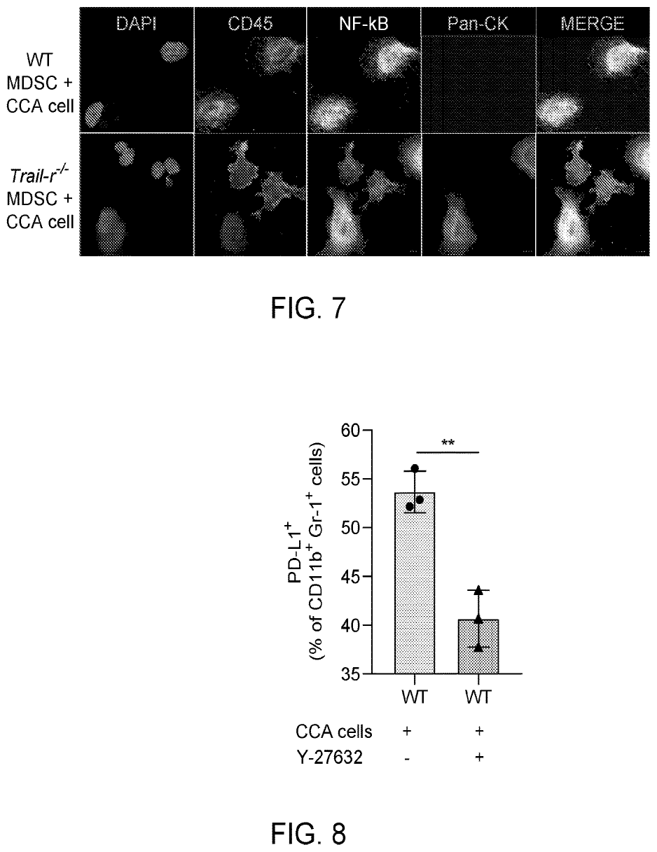

To delineate whether the observed effect of TRAIL signaling on MDSC proliferation was occurring via non-canonical TRAIL signaling, NF-kB activation was examined by assessing nuclear translocation of the NF-kB subunit p65 in WT MDSCs and Trail-r^{-/-} MDSC in the presence of TRAIL⁺ tumor cells. Trail-r^{-/-} MDSC had a reduction in the nuclear translocation of p65 compared to WT MDSCs when co-cultured with tumor cells, suggesting that TRAIL-TRAIL-R may augment MDSC proliferation via NFkB (FIG. 7). Furthermore, Trail-r^{-/-} MDSC had decreased expression of the immune checkpoint molecule PD-L1 compared with WT MDSCs (FIG. 6B). These results suggest that TRAIL signaling may augment the immunosuppressive capabilities of MDSCs. To determine whether the observed TRAIL-mediated increase in PD-L1 expression on MDSCs is occurring via ROCK1, ROCK pathway inhibition was employed. WT MDSCs were co-cultured with murine tumor cells and treated with vehicle or a ROCK inhibitor (Y-27632). A significant decrease in PD-L1 expression of WT MDSCs cocultured with murine CCA cells in the presence of the ROCK inhibitor was observed (FIG. 8). These results suggest that non-canonical TRAIL signaling increases PD-L1 expression on MDSCs in a ROCK1-dependent manner.

### Targeting TRAIL⁺ Cancer Cells

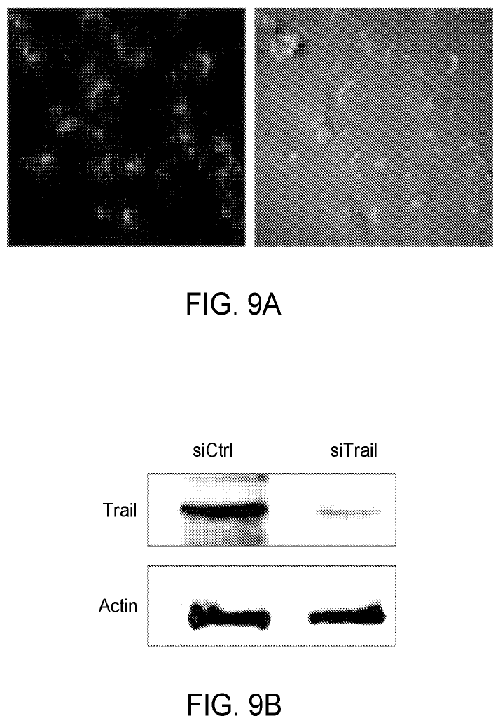

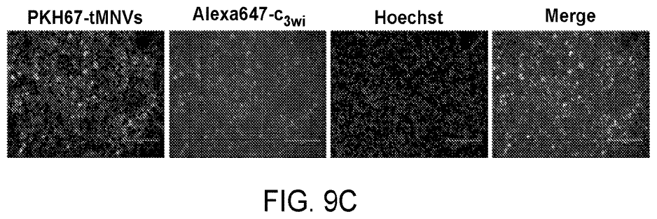

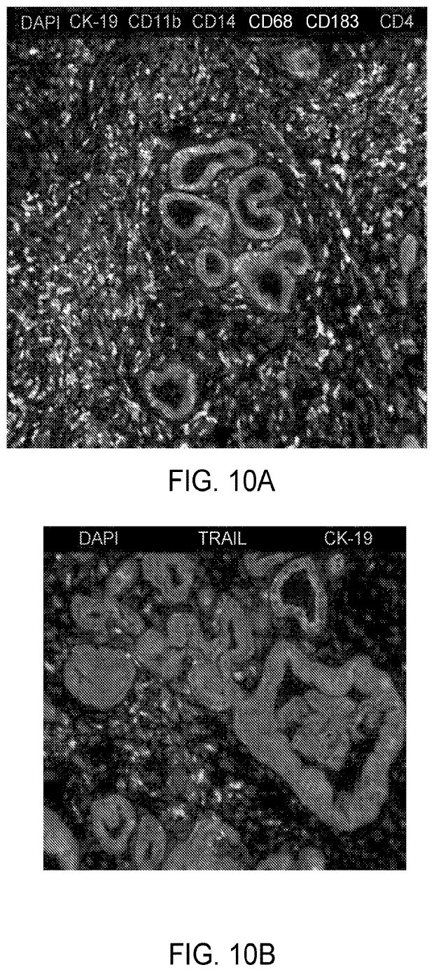

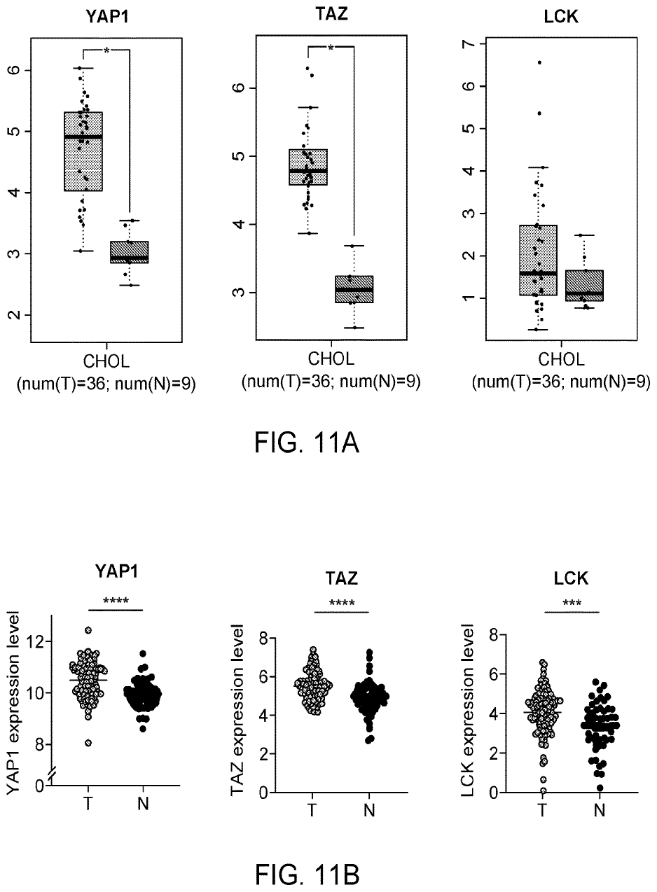

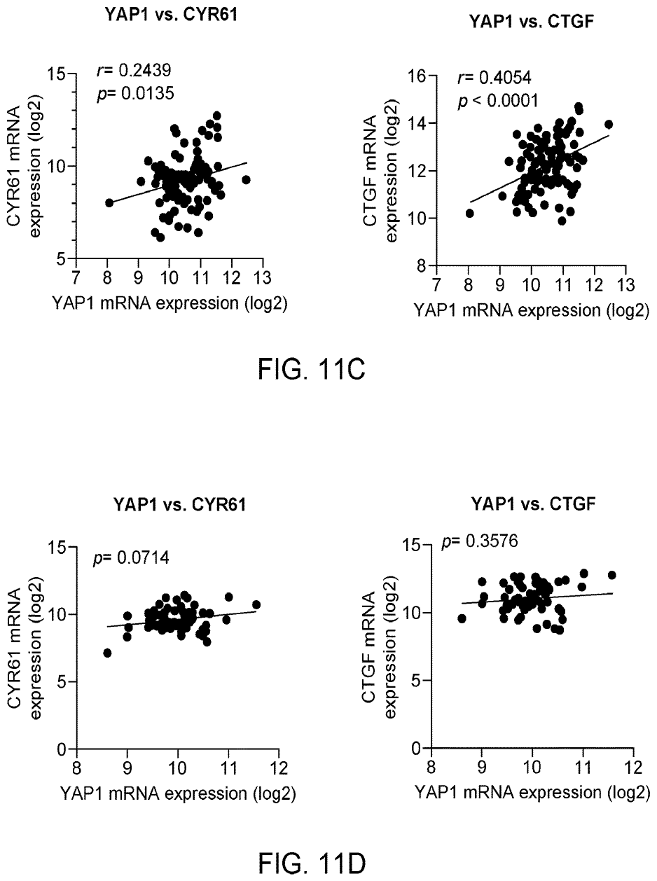

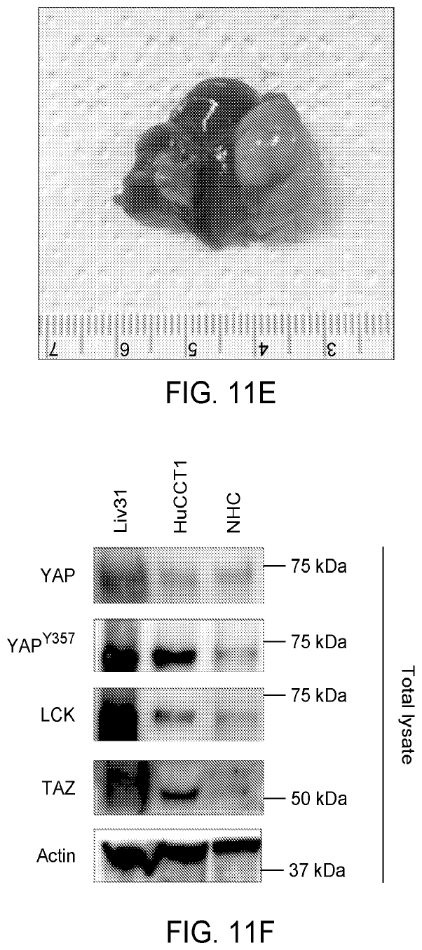

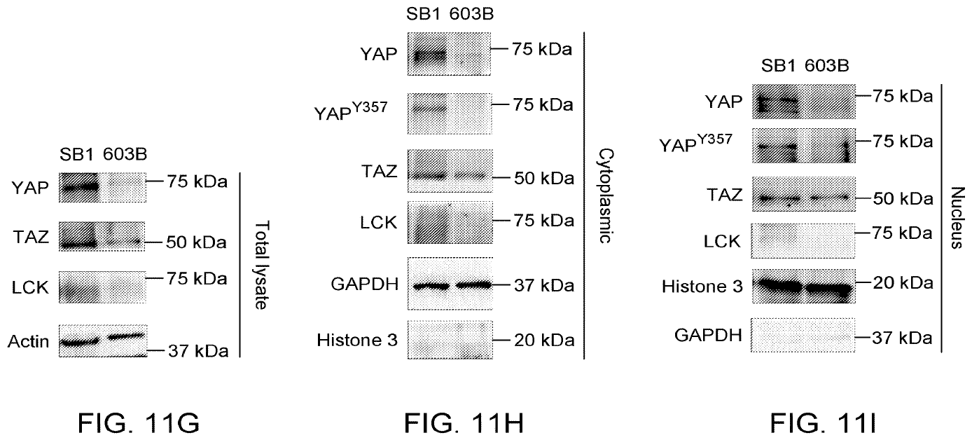

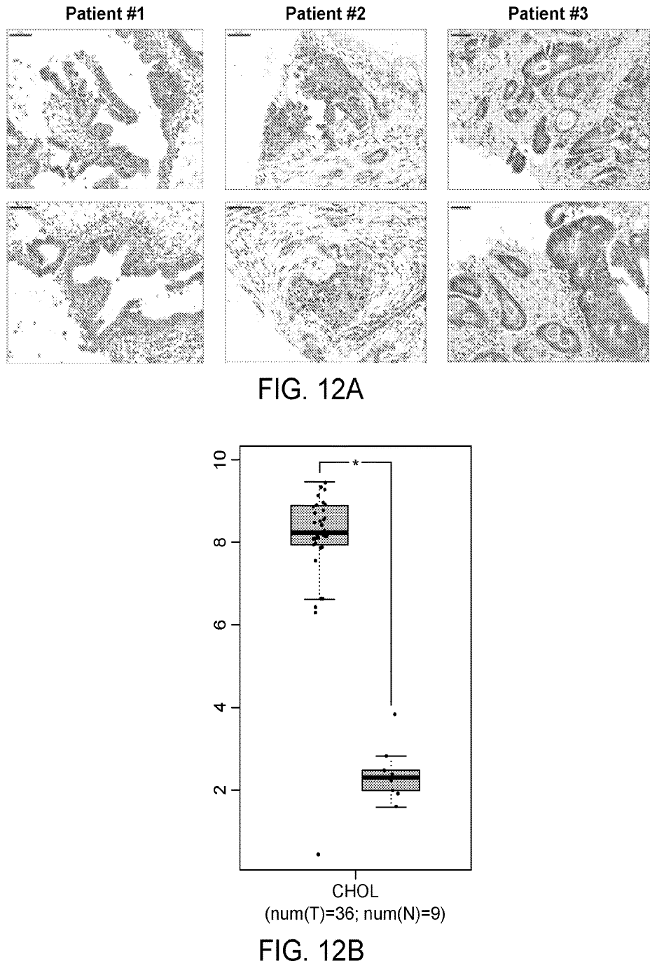

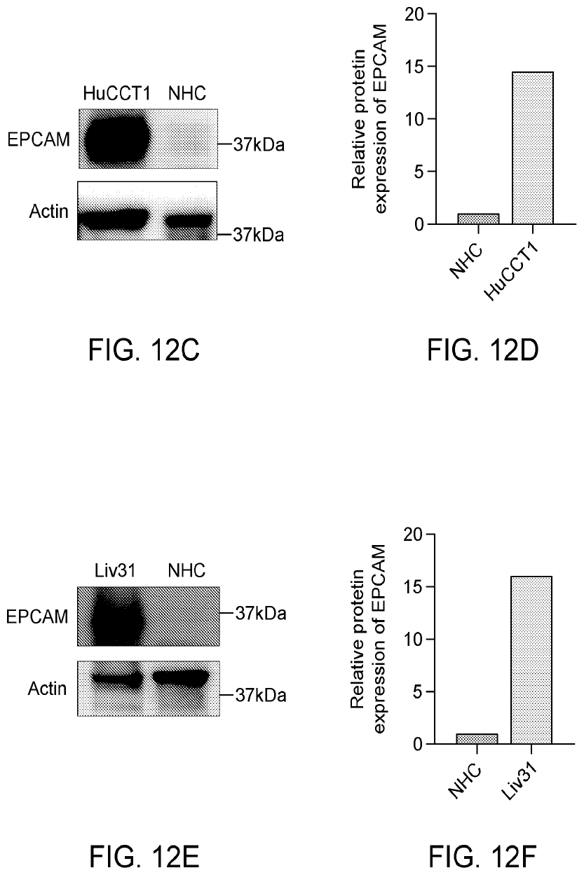

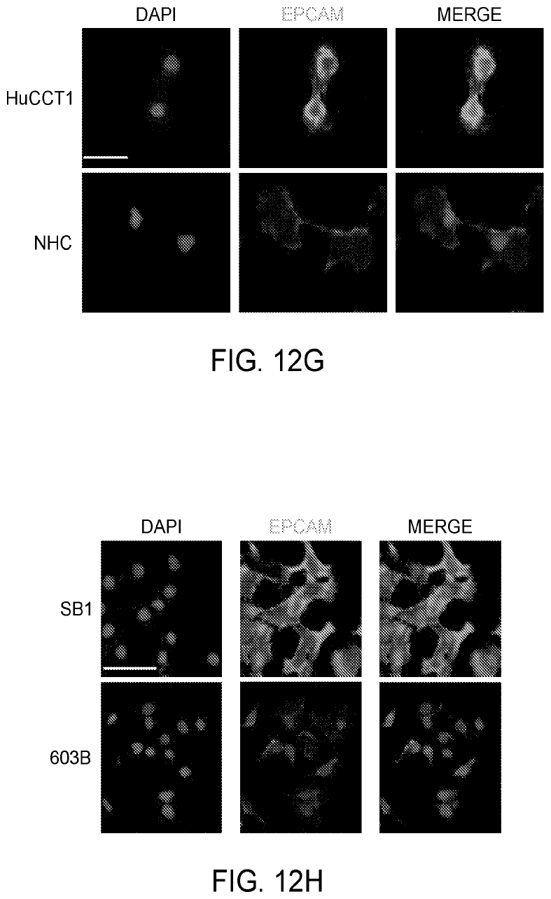

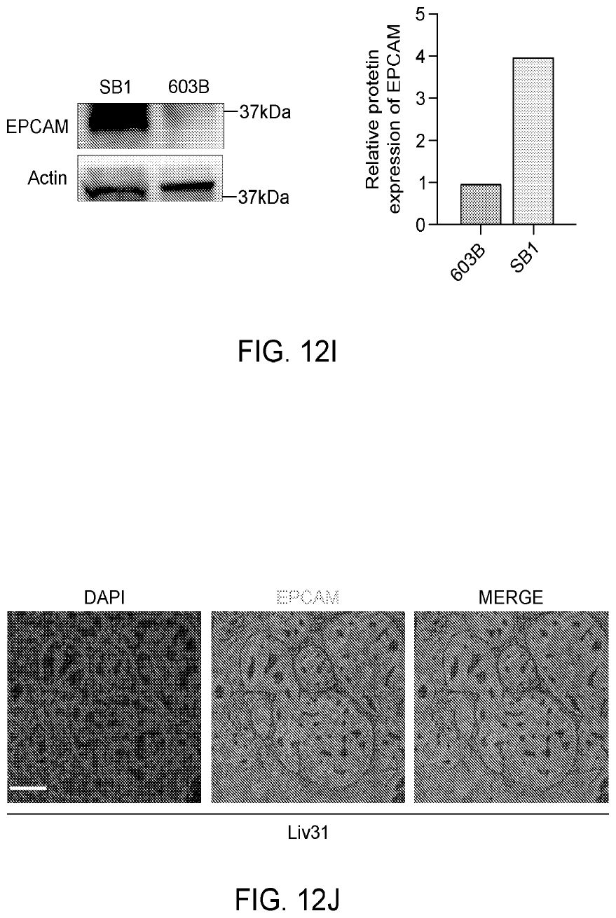

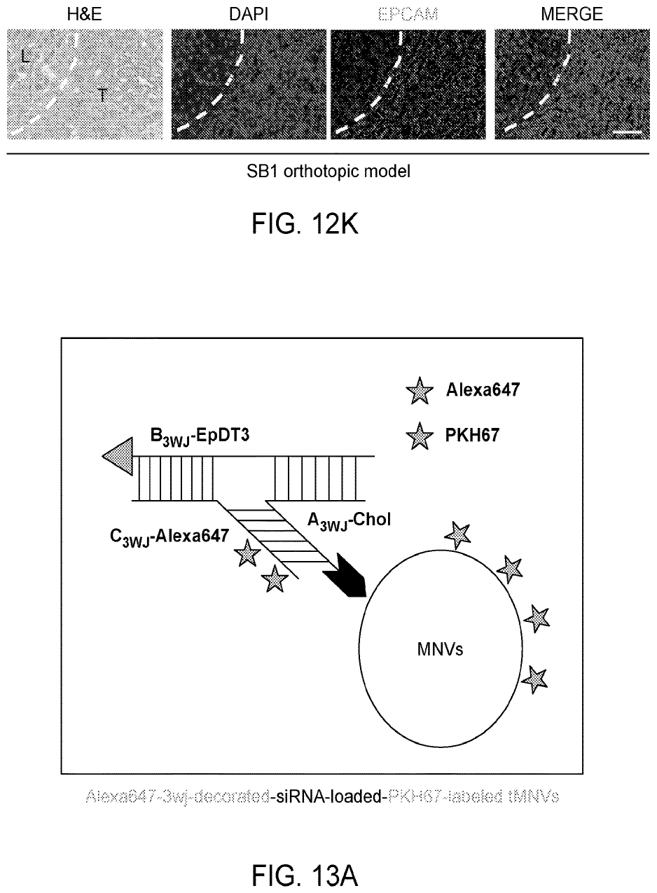

Milk-derived nanovesicles packaged (MDVN) with siTRAIL with an EPCAM (CCA cell marker) aptamer to target the siTRAIL directly to TRAIL⁺ CCA cells were generated. CCA cells took up the MDNV decorated with an aptamer targeting EPCAM (ET-MDNV) (FIG. 9A). Additionally, ET-MDNV loaded with siRNA for TRAIL efficiently reduced TRAIL protein abundance in murine CCA cells demonstrating on target downregulation (FIG. 9B). ET-MDNV were also administered to tumor bearing mice and demonstrated excellent biodistribution in the murine CCA tumors (FIG. 9C). A custom panel of 35 multiplexed immunofluorescence (MxIF). antibodies was created to dissect the spatial context of human TRAIL⁺ CCA cells, as it relates to myeloid cells, CTLs, and stromal components in the human CCA TIME. Using this panel, MxIF was conducted on surgical specimens of 20 treatment-naïve CCA patients who have undergone surgical resection (FIG. 10A), and an abundance of TRAIL⁺ CCA cells was identified (FIG. 10B).

### Example 2: Aptamer Directed, Nanovesicle-Mediated Transcriptome Targeting Improves Therapeutic Response and the Tumor Immune Microenvironment in Preclinical Cholangiocarcinoma Models

This Example describes using nanovesicles loaded with siRNAs that targeting YAP and/or TAZ and an upstream regulator, LCK, to treat cancer.

### Results

### EpCAM, YAP, TAZ, and LCK are Elevated in CCA Tumors and Preclinical Models

Tissue level evaluation of YAP1 and WWTR1 mRNA levels, encoding YAP and TAZ (YAP/TAZ), respectively, in both TCGA and GEO databases (GSE26566) demonstrated elevated levels in CCA tissues compared to normal tissue controls (FIG. 1A-B). Increased YAP levels were also positively correlated with expression of the YAP-target genes CTGF and CYR61 in tumor tissue, but not in surrounding normal liver, suggesting co-transcriptional activity of YAP and TAZ in CCA tumors (FIGS. 1C-D). It was next examined whether YAP, TAZ, and LCK protein levels in multiple preclinical CCA models compared to normal cholangiocytes. The human CCA cell line HuCCT1, the murine CCA cell line SB1, and tumor tissue from a patient-derived xenograft, Liv31 were evaluated. HuCCT1 was originally isolated from the peritoneal fluid of a patient with metastatic intrahepatic cholangiocarcinoma, SB1 was generated from an activated YAP and AKT murine CCA model, and Liv31 was generated from a patient with intrahepatic cholangiocarcinoma and carries both an FGFR2-CCDC6 fusion and PTPRB mutation (FIG. 1E). All CCA models demonstrated increases in YAP, TAZ, and LCK as compared to species-specific normal controls. Furthermore, evaluation of pYAP^{Y357} levels, a YAP activation mark associated with LCK activity, demonstrated increases all three models. Finally, given the cytoplasmic sequestration of YAP and TAZ associated with deactivation of these molecules, the cytoplasmic and nuclear fractions in the models were evaluated, and increases in YAP, pYAP^{Y357}, and LCK were noted in the nuclear fraction of the cancer models as compared to controls. Levels of TAZ were similar in the cytoplasm and nuclear compartment in the models. Given that the cell surface glycoprotein EpCAM had been utilized as the aptamer target in HCC tumor models EpCAM levels in publically available databases, resected CCA tumor specimens, and in preclinical models were evaluated. Intense expression of EpCAM was observed in CCA human tissues by immunohistochemistry (FIG. 2A). In the TCGA dataset, EPCAM mRNA was significantly elevated in CCA tumor tissue as compared to normal liver tissues (FIG. 2B). The cell line and PDX models were then assessed by RT-PCR, immunoblot, and immunofluorescence. All preclinical models demonstrated markedly increased EpCAM expression as compared to species specific normal controls. Finally, it was assessed whether EpCAM expression was altered in the SB1 model after implantation in the livers of syngeneic mice, and noted continued high levels of EpCAM expression. These data supported the use of EpCAM as the aptamer target in CCA preclinical models.

### MNVs can be Delivered to CCA In Vitro and In Vivo

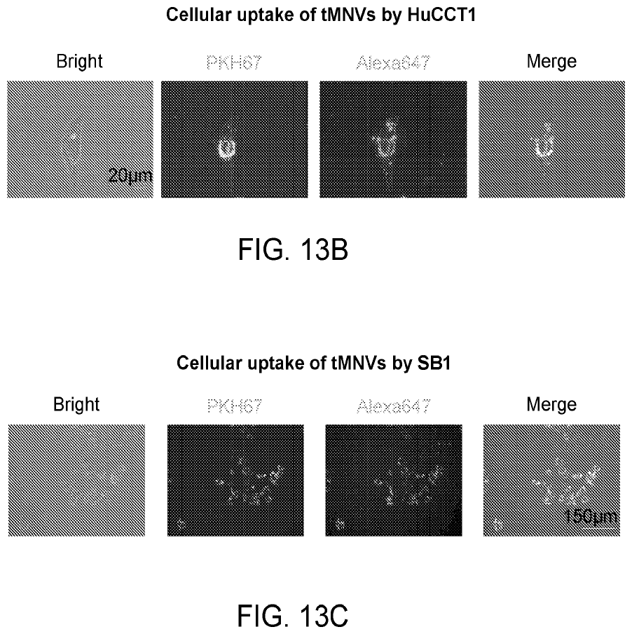

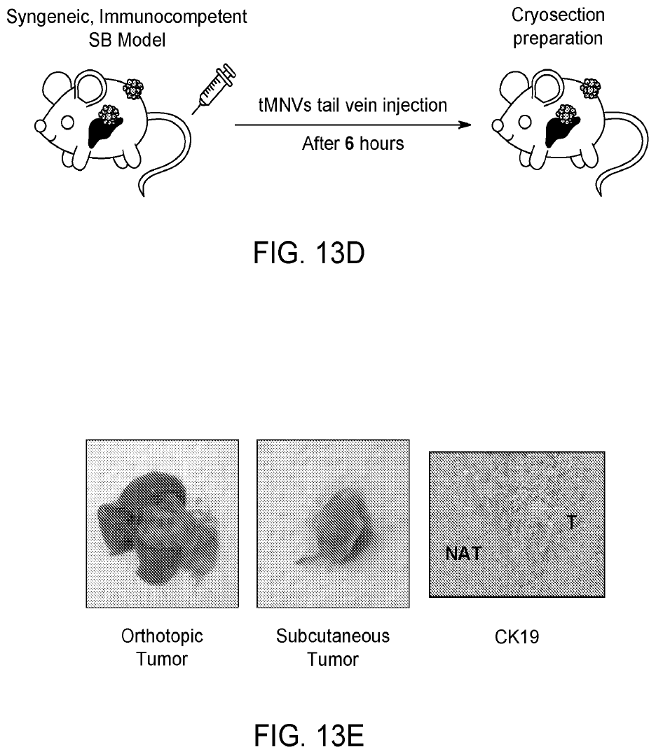

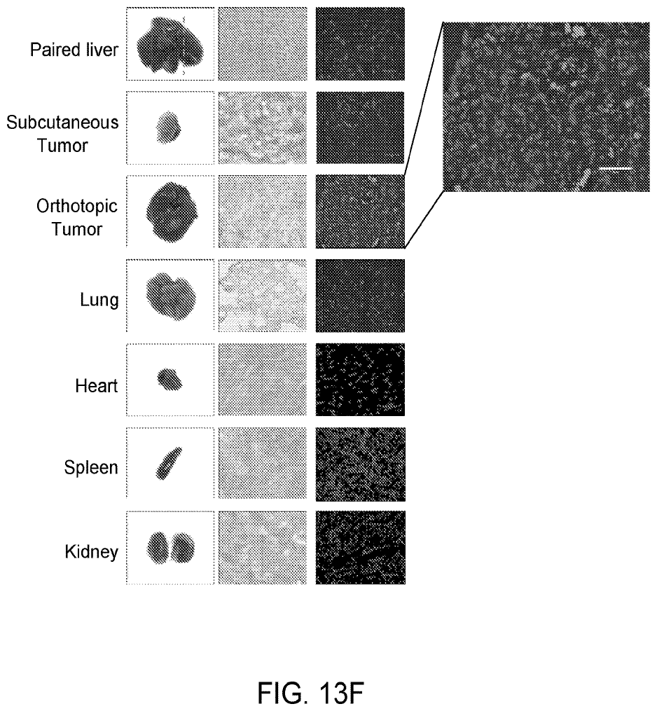

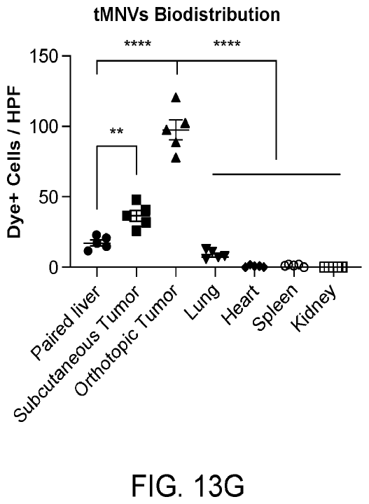

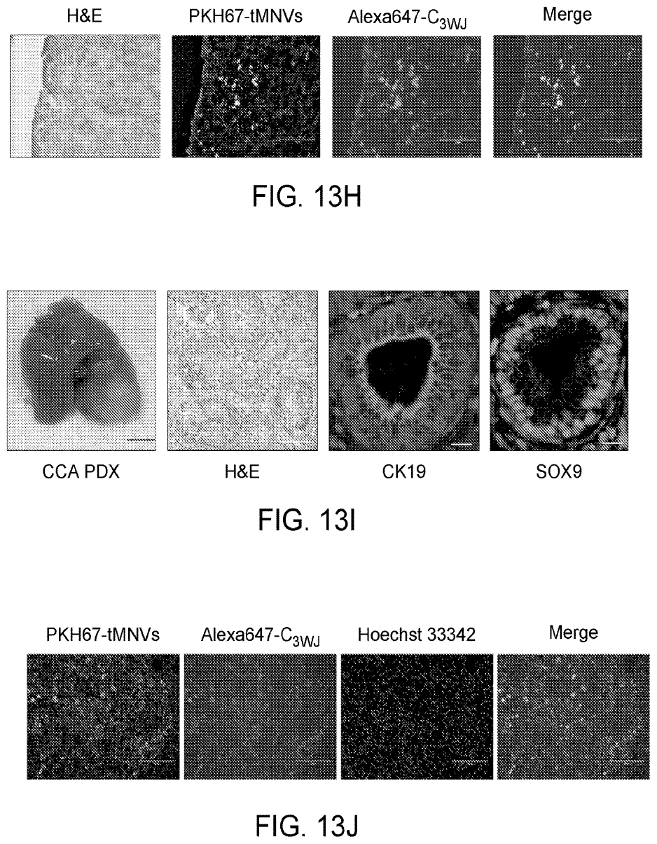

To assess milk-derived nanovesicle (MNV) delivery of siRNA as a targeting strategy in CCA RNA nanoparticles containing a validated aptamer against EpCAM were utilized. The engineered RNA nanoparticles can be inserted into the MNV lipid bilayer through a conjugated cholesterol-triethylene-glycol (TEG). The engineered RNA nanoparticles also contain an Alexa647 fluorophore for tracking purposes. The design of RNA nanoparticles/MNV complex (referred to as tMNVs) is illustrated in FIG. 13A. Incubation of tMNVs loaded with the fluorescent dye PKH67 with the SB1 and HuCCT1 cell lines demonstrated uptake by fluorescence microscopy (FIG. 13B-13C). It was then assessed whether inclusion of the aptamer improved delivery of MNVs and whether tMNVs can concentrate in SB1 tumors relative to surrounding non-cancerous liver tissue. Paired murine CCA tissue and normal liver tissue were cut into uniform 4 mm³ tissue plugs using biopsy punch and placed in 96-well plates. tMNVs or “bare” MNVs were loaded with the fluorescent dye PKH67 and incubated with the tissue sections. Flowcytometry after 48 hours incubation demonstrated uptake of MNVs which was higher in the tumor tissue as compared to normal surrounding liver, and that this was improved in tumor tissue by addition of the aptamer (tMNVs). To investigate the biodistribution of tMNVs in vivo, 1×10¹¹ tMNVs were systemically delivered by tail vein injection into C57BL/6j mice bearing both subcutaneous and orthotopic SB1 tumors (FIG. 13D). Six hours following administration, tumors and organs were harvested from mice and prepared snap-frozen sections. The highest fluorescent signals were detected in orthotopic SB1 tumors, followed by the subcutaneous tumors, and then the normal surrounding liver (FIG. 13F-13G). Minimal fluorescence was noted in the lung and none in the heart, spleen, and kidneys (FIG. 13G). Evaluation of the PKH67 signal as compared to the Alexa647 signal demonstrated excellent overlap, suggesting that in vivo administration did not lead to cleavage of the aptamer complex from the MNV (FIG. 13H). Given the concentration of tMNVs in orthotopic tumor as compared to subcutaneous we then established Liv31 PDX in an orthotopic location (FIG. 13I). After intravenous delivery of tMNVs, similar to syngeneic murine model, high fluorescent signals were detected in snap-frozen PDX tumor sections (FIG. 13J). Overall, these results indicate that tMNVs can be taken up CCA cells within liver, and have selectivity over normal surrounding tissue

tMNV Delivered siRNA Reduces YAP/TAZ/LCK Levels Sensitizes CCA to Gemcitabine/Cisplatin In Vitro.

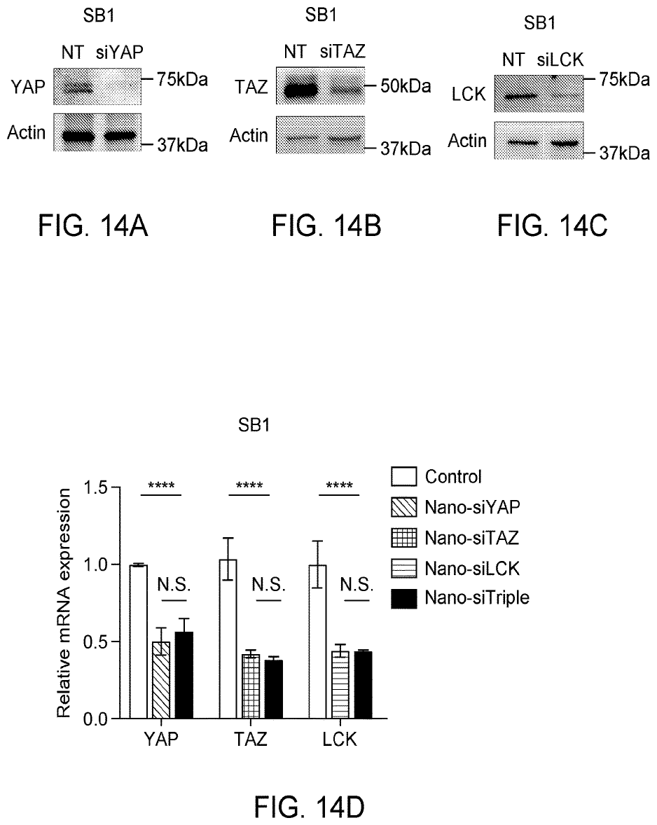

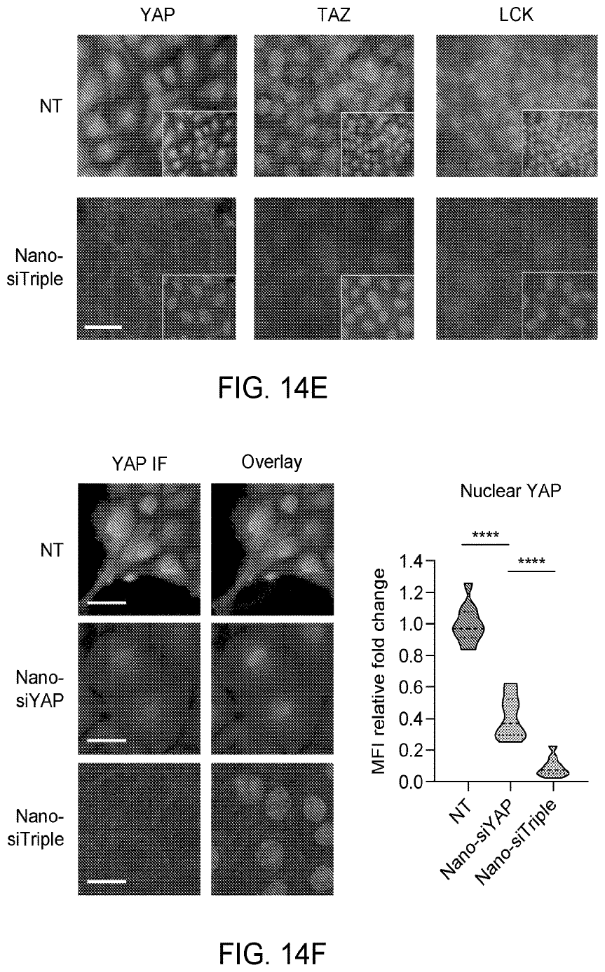

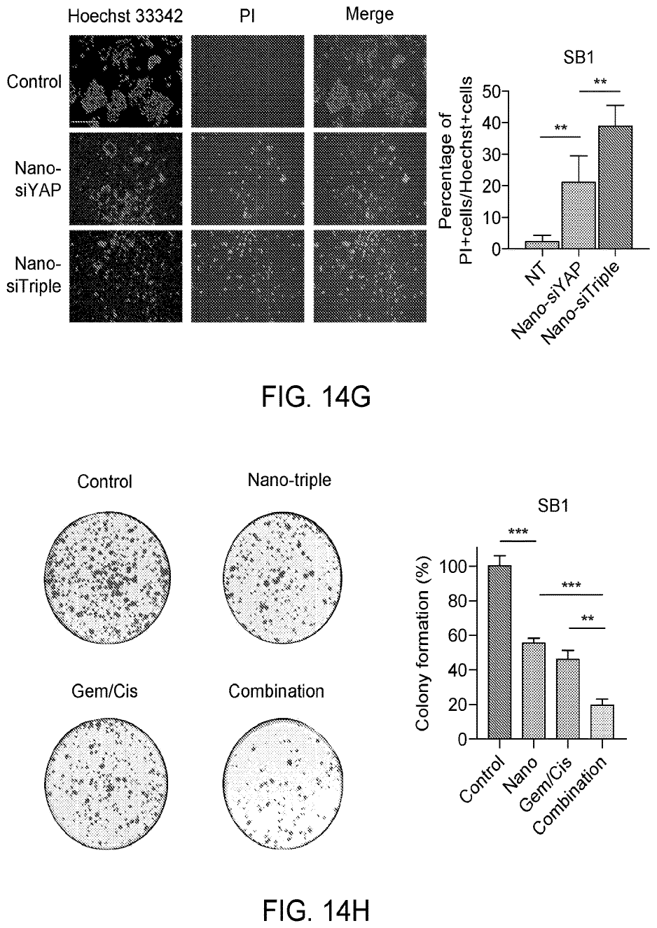

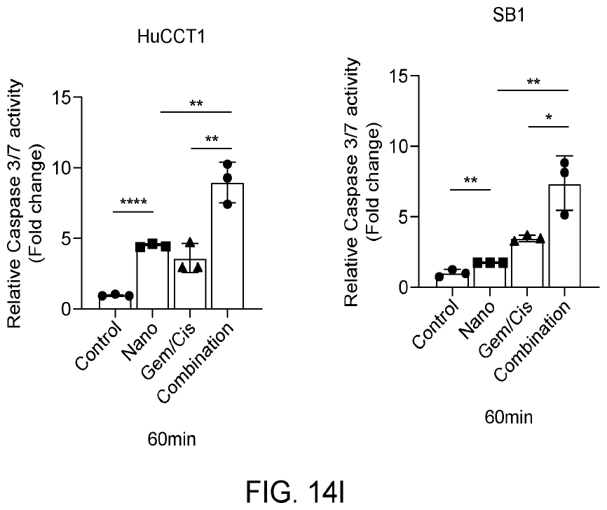

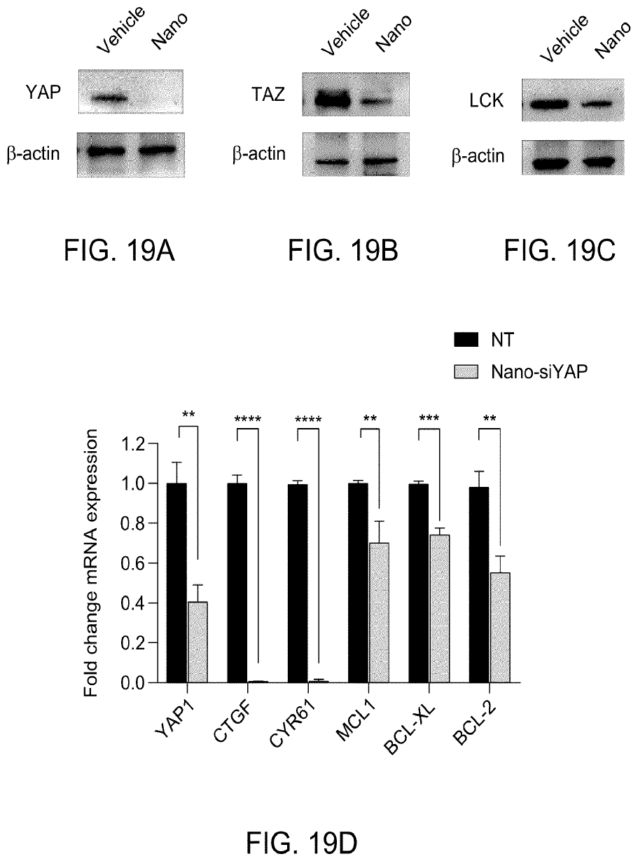

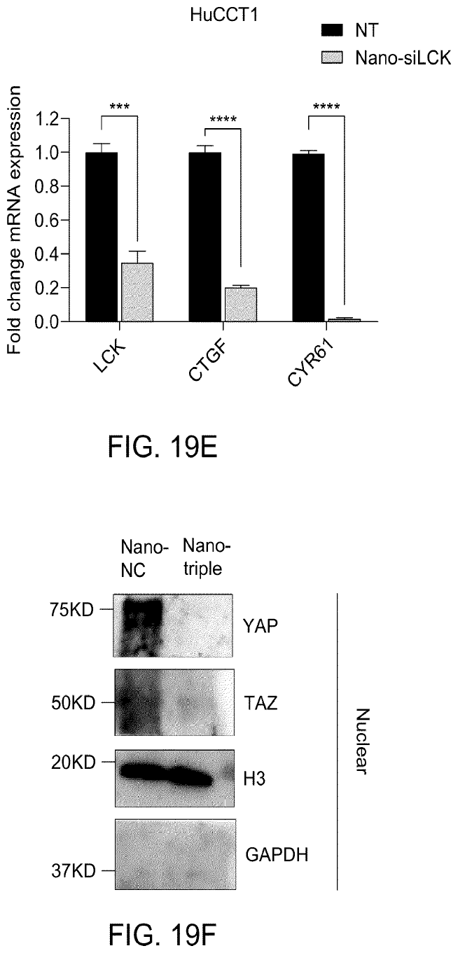

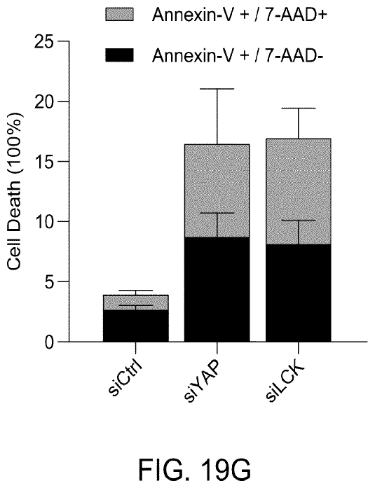

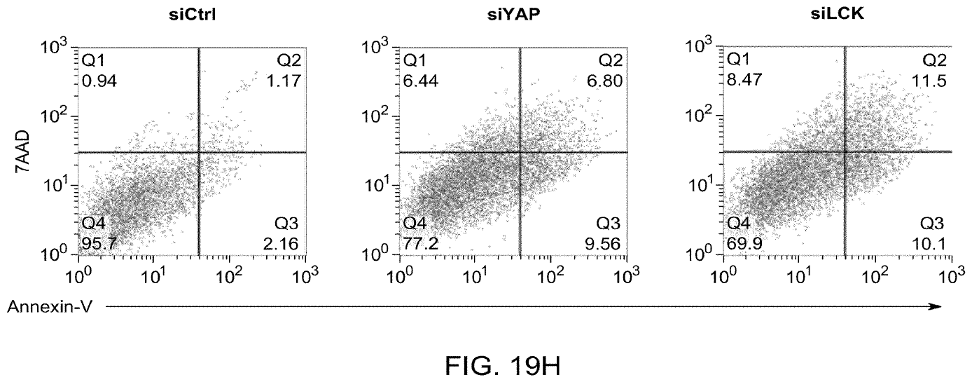

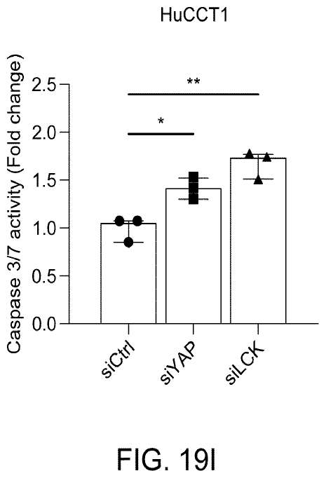

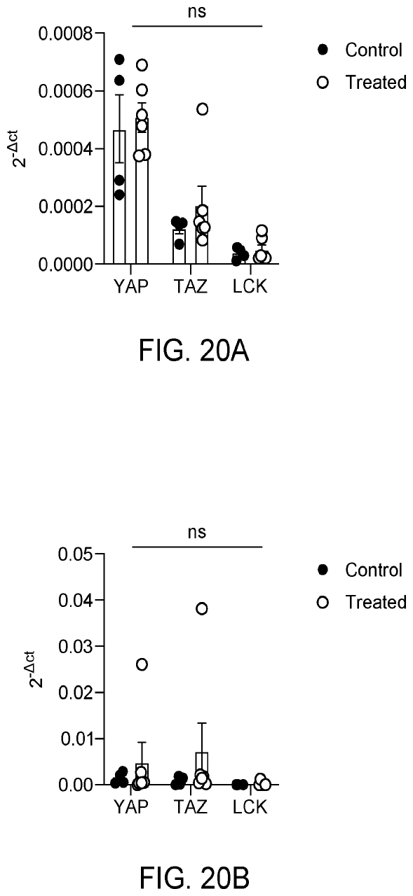

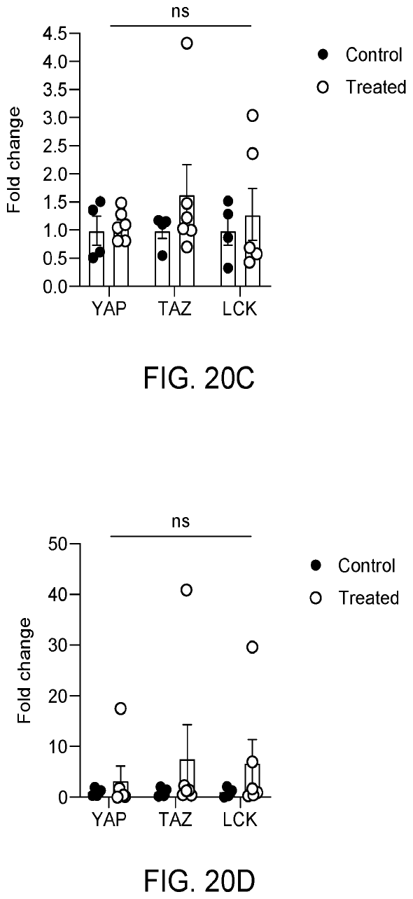

To evaluate the knockdown effects of siRNA-loaded tMNVs in CCA, tMNVs containing species-specific siRNAs targeting YAP, TAZ, or LCK were generated. It was found that incubation with gene-targeted tMNVs led to decreased total YAP, TAZ, or LCK protein level in both SB1 and HuCCT1 cells after 48 hours (FIG. 14A-14C, FIG. 19A-19C). To investigate whether tMNVs can be loaded with multiple gene-targeted siRNAs at the same time, tMNVs were loaded with siYAP, siTAZ and siLCK (referred to as siTriple) and it was found that tMNVs loaded with the same concentration of siTriple (5 M) had similar degrees of target knockdown compared to the tMNVs loaded with siRNAs targeting YAP, TAZ, or LCK separately (FIG. 14D). Immunofluorescence staining further confirmed the knockdown effects mediated by tMNVs loaded with siTriple (FIG. 14E). In line with the previous finding that LCK knockdown induced YAP translocation from the nucleus to the cytoplasm in CCA, it was observed that tMNVs loaded with siTriple decreased nuclear YAP compared to tMNVs with siYAP alone (FIG. 14F). And tMNVs loaded with siTriple treatment also led to decreased transcriptional activity as measured by mRNA expression of canonical YAP target genes. Incubation of CCA cells with siYAP led to increases in cell death as marked by PI staining, and incubation with siTriple significantly increased the cell death over siYAP alone (FIG. 14G). Since the efficacy of first-line chemotherapy regimens remains modest in CCA, SB1 cells were treated with siTriple-loaded tMNVs combined with gemcitabine and cisplatin (gem/cis), and it was found that attenuation of YAP, TAZ, and LCK potentiated the effects of gem/cis in SB1 cells (FIG. 14H-14I). Together these results suggest that siRNA delivered by tMNVs effectively mediates YAP, TAZ, and LCK knockdown in vitro and knockdown of these targets can improve response to gem/cis.

tMNVs Mediates YAP/TAZ/LCK Knockdown in Both Murine and PDX CCA Models

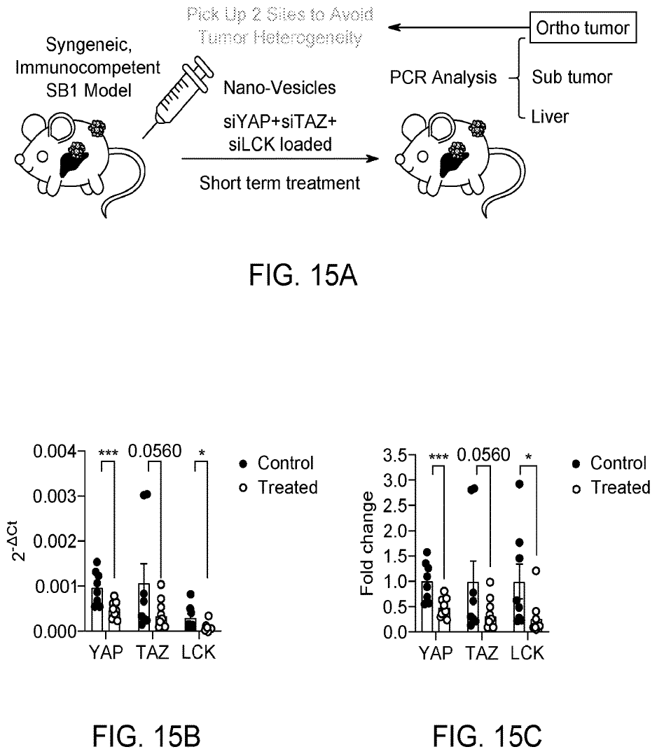

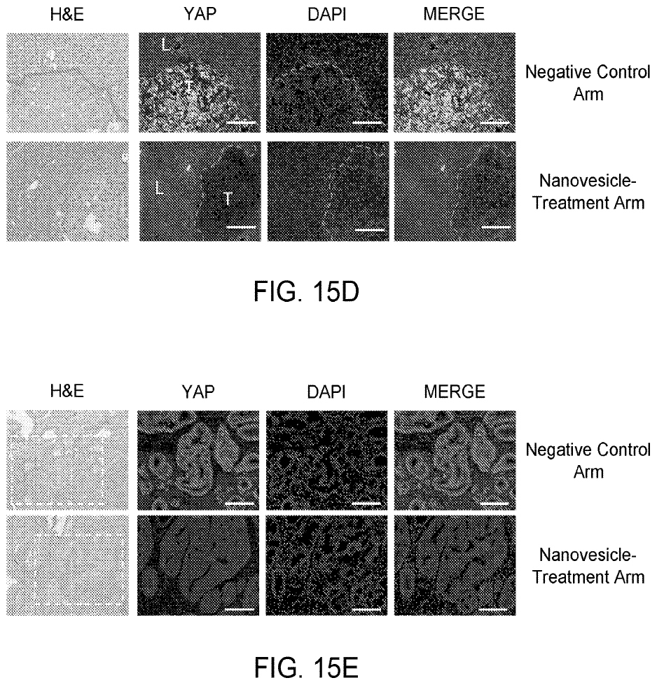

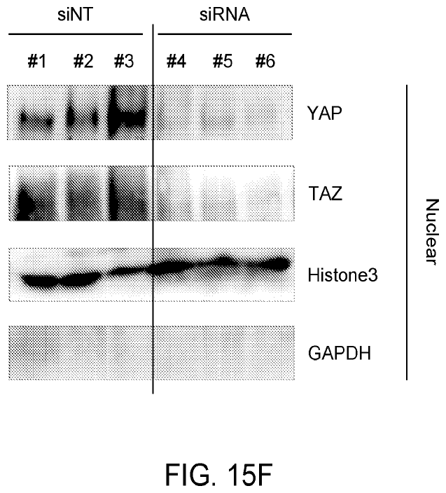

It was next sought to evaluate the knockdown effects mediated by tMNVs in multiple CCA models in vivo. To explore this, one week course of treatment was administered. C57BL/6 mice bearing both subcutaneous and orthotopic SB1 tumors were treated with tMNVs (2×10¹² particles/body) one to three times via tail vein injection (n=6). Paired liver, subcutaneous, and orthotopic SB1 tumors from the same mice were then harvested after the treatment. Specifically, two sites of orthotopic tumors were collected from each mouse to minimize the effects confounded by tumor heterogeneity (FIG. 15A). Quantitative real time PCR was performed to determine the YAP/TAZ and LCK mRNA levels in different sites of this model. In orthotopic SB1 tumor sites, tMNVs decreased YAP, TAZ and LCK mRNA levels as compared with control group (FIG. 15B-15C). Cohort analysis was performed based on the number of doses of tMNVs treatment as compared with control group, and it was found that three doses of tMNV reached the highest knockdown efficiency (62.6%-70.5% of YAP, 66.3%-92.3% of TAZ and 60.8%-90.4 of LCK) compared to the mice without treatment. Two separate cohorts of treatments were initiated in both SB1 model and PDX CCA model. Total seven doses of tMNVs were administered every two days for two weeks. And two days after the final dose of tMNVs, orthotopic tumors were harvested and immunofluorescence staining results showed that YAP protein level were strikingly decreased in both SB1 and Liv31 tumor sections (FIG. 15D-15E). Moreover, nuclear extract was isolated from bulk tumors, and immunoblot assays further confirmed that nuclear YAP/TAZ protein levels were dramatically decreased compared to the control groups (FIG. 15F), supporting the therapeutic application of tMNVs in multiple CCA models in vivo.

Combined YAP/TAZ/LCK Knockdown Through tMNVs with GEMICS Results in CCA Regression.

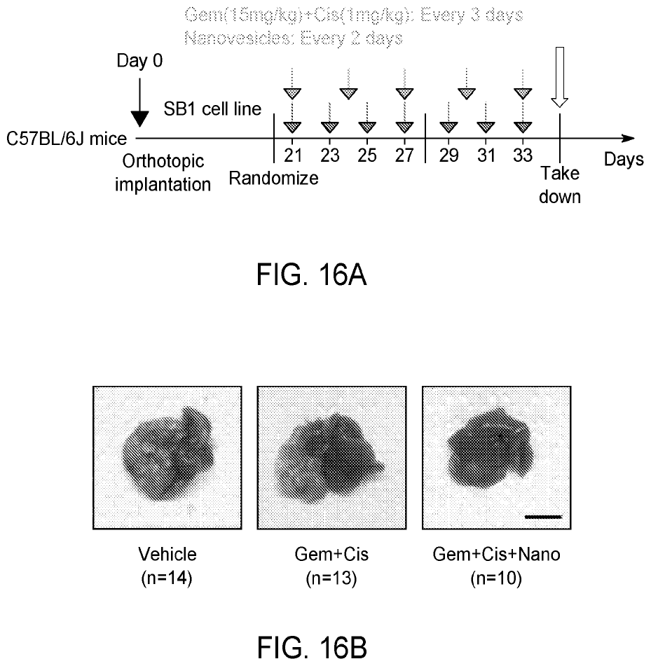

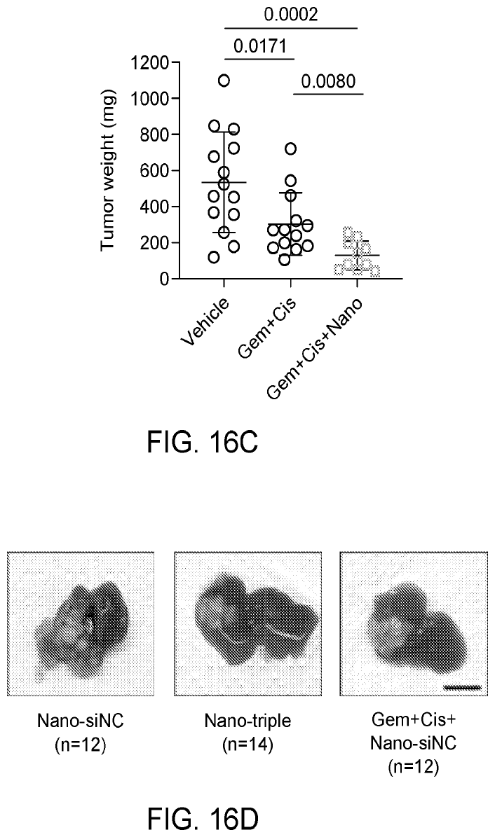

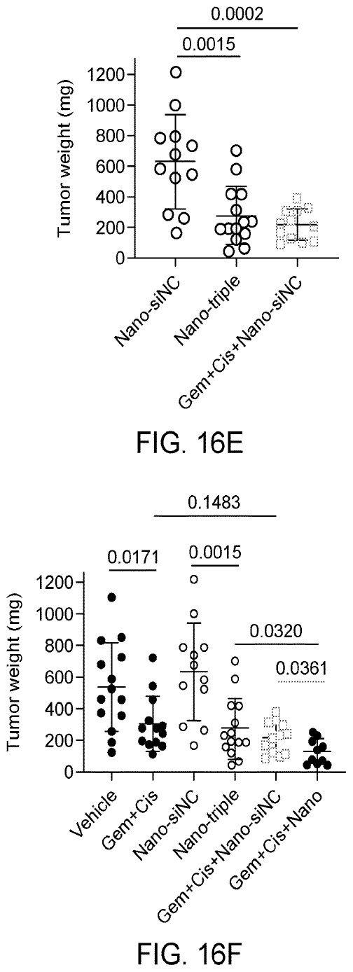

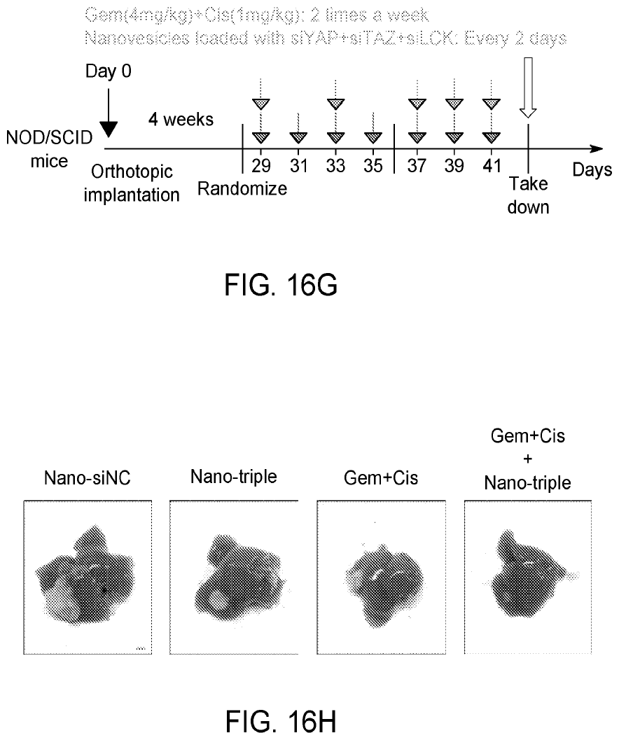

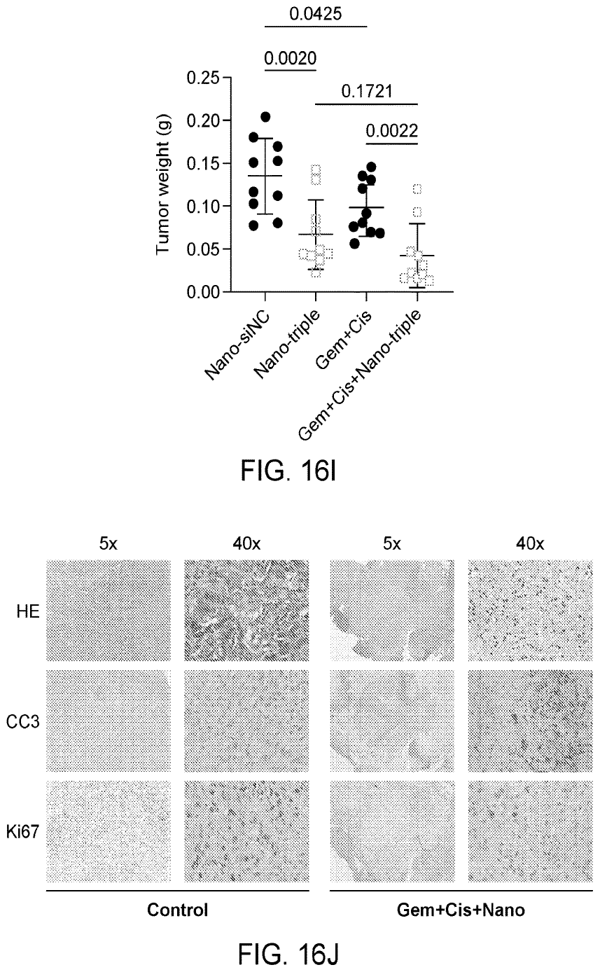

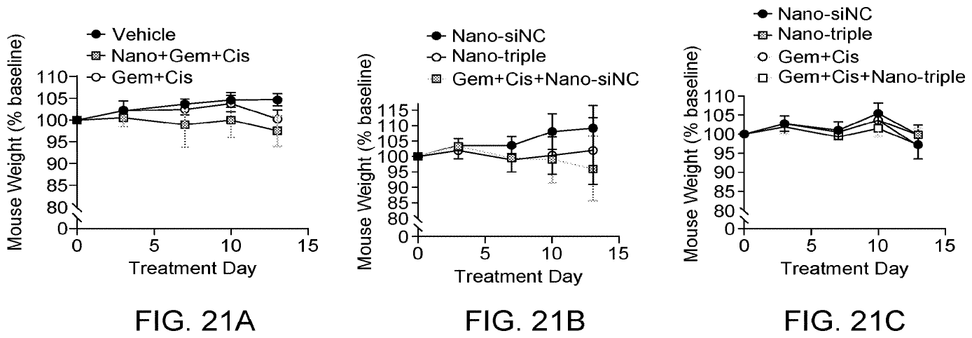

Given the synergized effects with chemotherapy in vitro and knockdown effects in vivo, it was next sought to evaluate the efficacy of combination treatment in SB1 model. Three weeks following SB1 cells implantation, C57BL/6 mice bearing SB1 orthotopic tumors were treated with gemcitabine (15 mg/kg) plus cisplatin (1 mg/kg) every three days, and siTriple-loaded tMNVs (2×10¹² particles) every two days (FIG. 16A). For the first combination studies, mice were treated with normal saline (refer to vehicle) every three days as a control group since normal saline was utilized to dilute gemcitabine and cisplatin. Compared with chemotherapy alone, the combination of siTriple-loaded tMNVs significantly decreased the growth of SB1 tumors (FIG. 16B-16C), consistent with the in vitro results (FIG. 14H). For the second combination studies, mice were treated with tMNVs loaded with scramble siRNAs (refer to siNC) as a control group and combined with chemotherapy. Either treatment with siTriple-loaded tMNVs or chemotherapy modestly suppressed SB1 tumor growth (FIG. 16D-16E). When combined the analysis of those combination studies, it was found that there was no difference in tumor weight between chemotherapy-treated and chemotherapy combined with siNC-loaded tMNVs-treated groups (FIG. 16F). tMNVs treatment was well tolerated overall based on the lack of weight change between vehicle-treated and tMNVs-treated groups (FIG. 21A-21C). To investigate whether siTriple-loaded tMNVs can modulate sensitivity to chemotherapy in the PDX CCA model, NOD/SCID mice bearing orthotopic Liv31 tumors were treated with gemcitabine (4 mg/kg) plus cisplatin (1 mg/kg) every three days, and siTriple-loaded tMNVs (2×10¹² particles) every two days or siNC-loaded tMNVs every two days (FIG. 16G). Similar to murine CCA model, the combination therapy strikingly suppressed the growth of Liv31 tumors with no statistical change of weight compared with other groups (FIG. 16H-16I, FIG. 21C).

### Knockdown of YAP/TAZ/LCK Increases Reactive CTLs and NK Cells in CCA.

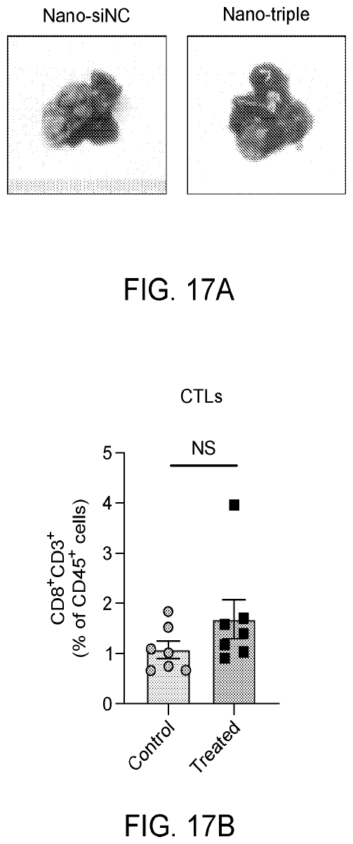

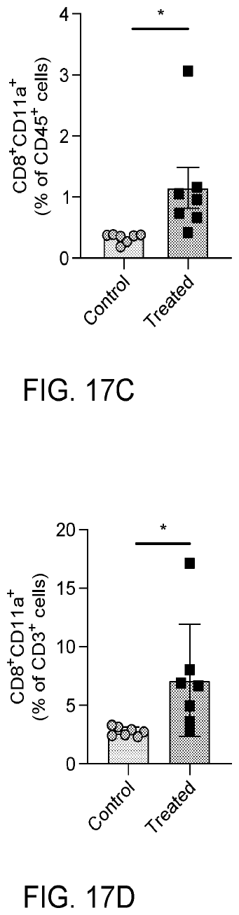

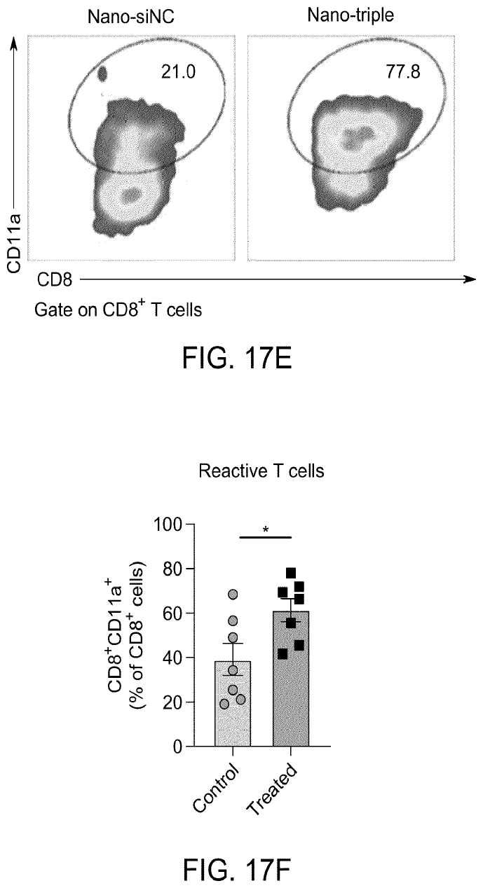

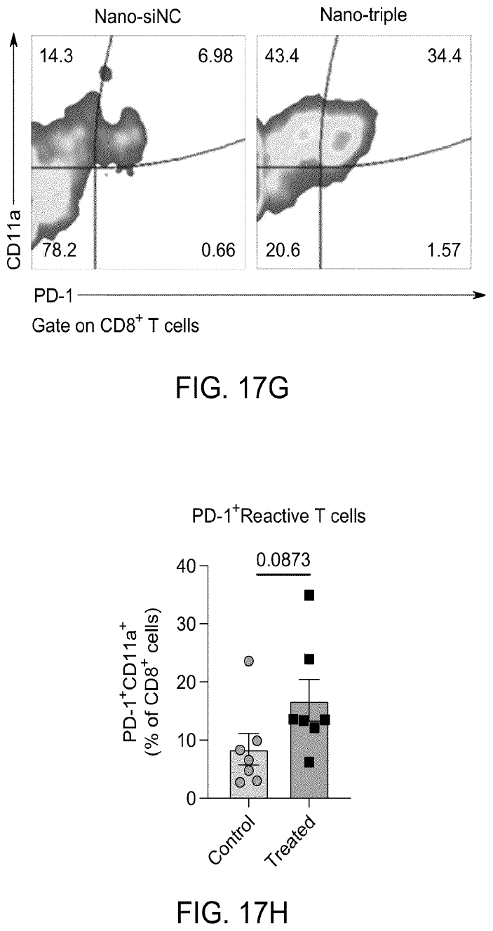

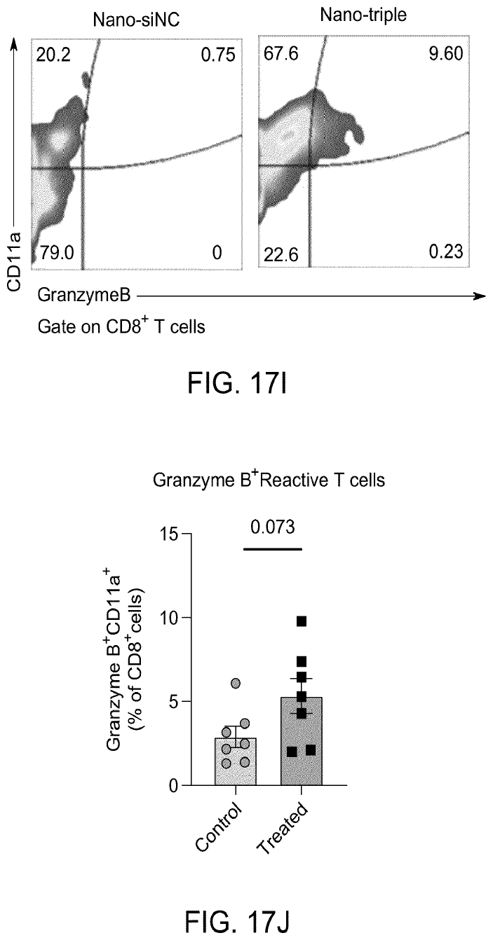

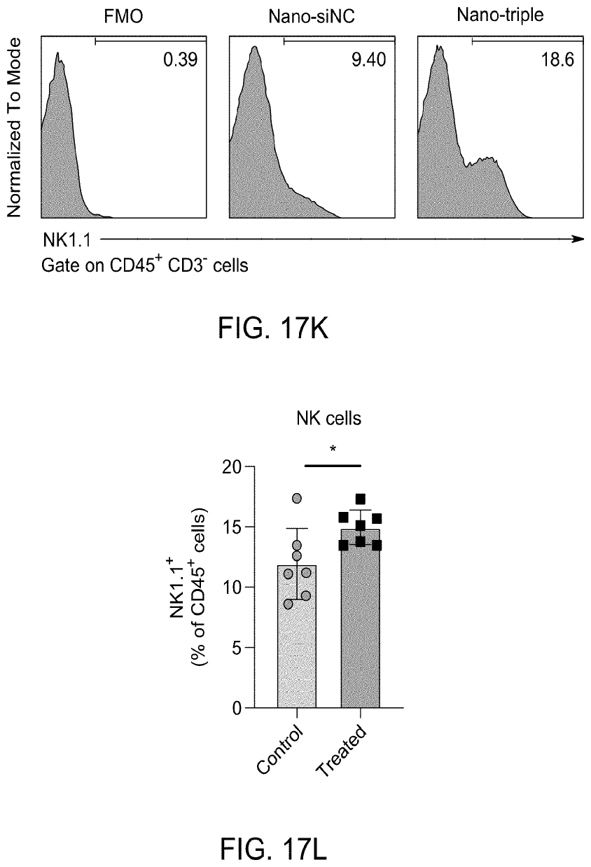

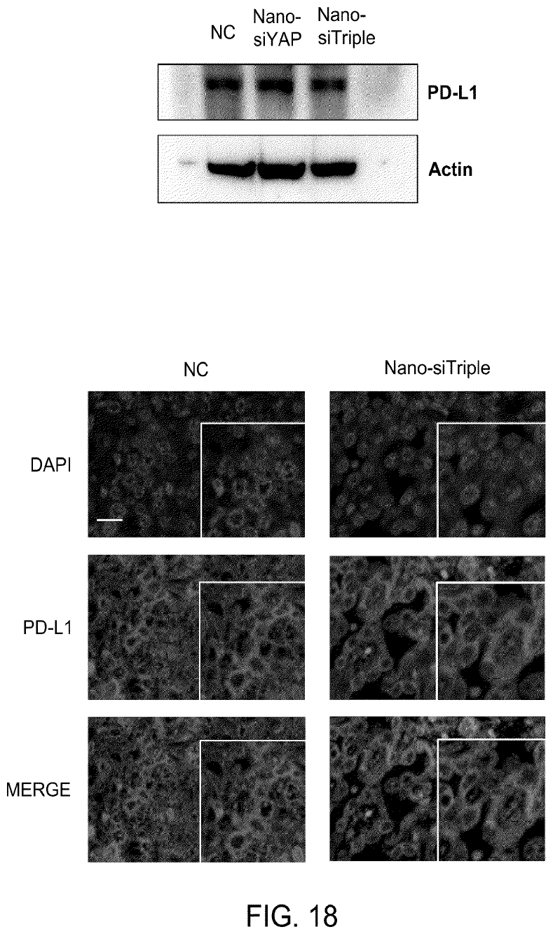

Utilizing an immunocompetent, syngeneic SB1 model in C57BL/6 mice, changes in tumor-infiltrating lymphocytes in CCA after directly knocking down YAP/TAZ were analyzed in vivo (FIG. 17A). Although it was observed that there were no change of tumor-infiltrating CD8⁺ T cells in quantity between siTriple-loaded tMNVs group and siNC-loaded tMNVs group (FIG. 17B), it was found that siTriple-loaded tMNVs significantly increased reactive CTLs (FIG. 17C-17F), as demonstrated by CD11a expression. High levels of CD11a expression on CD8⁺ T cells are essential for CTLs activation and trafficking, and flowcytometric analysis showed that reactive CTLs were increased and contributed to a large proportion of total CTLs (CD45⁺CD3⁺CD8⁺) after YAP/TAZ knockdown (approximately 60% of all CTLs, FIG. 17E-17F). Moreover, PD-1 and Granzyme B were trended towards increase on reactive CTLs when compared to control group (p=0.087 and p=0.073 respectively, FIG. 17G-17J). On the other hand, another key antitumor effector, natural killer (NK) cells were also increased after the treatment (FIG. 17K-17L). Overall, these results indicated that YAP/TAZ knockdown augment anti-tumor immunity through recruiting reactive CTLs and NK cells into the CCA TME.

### Materials & Methods

### Cell Lines and Culture

The human CCA cell line HuCCT-1, mouse CCA cell line SB1, normal human cholangiocyte cell line, and SV40-transformed, nonmalignant mouse cholangiocyte cell line (603B) were cultured in DMEM containing 10% FBS, 100 units/mL of penicillin and 100 μg/mL streptomycin at 37° C. and 5% CO₂. SB1 cell line was derived from a syngeneic YAP-driven CCA model and expresses flag-tagged YAP^{S127A} and myr-AKT. Cell lines were authenticated using short tandem repeat profiling and used at low passage numbers.

### Immunoblot Assays

Whole-cell lysates were collected using cell lysis buffer (Cell Signaling Technology) with phosphatase inhibitors (Roche), protease inhibitors (Roche), and 1 mM PMSF. Nuclear and cytoplasmic protein extracts were prepared using nuclear and cytoplasmic extraction kit (Thermo Fisher Scientific; #78833) based on manufacturer's instructions. Proteins were eluted and transferred onto nitrocellulose membranes. Blots were blocked in 5% BSA in TBST and incubated with primary antibodies overnight at 4° C. Primary antibodies used were YAP (Cell Signaling Technology, 4912S, 1:1000), EPCAM (Abcam, ab71916, 1:1000), Histone 3 (Cell Signaling Technology, 4499s, 1:2000), YAP357 (Abcam, Ab62571, 1:1000), TAZ (Cell Signaling Technology, 4883S, 1:1000), LCK (Abcam, ab189933, 1:1000), Actin (Santa Cruz, sc-47778, 1:1000) and GAPDH (Santa Cruz, sc-32233, 1:1000). Secondary HRP antibodies for mouse (Invitrogen, 62-6520), rabbit (Invitrogen, #31460) were used at a concentration of 1:2000. Proteins were visualized with ECL (GE Healthcare Life Sciences) chemiluminescence. Relative protein expression were evaluated and quantified using ImageJ (Fiji).

### Immunofluorescence Assays

Immunofluorescence staining was performed using standard protocol. Briefly, tissues for immunofluorescence were collected after overnight fixation in 4% PFA at 4° C. Cells were plated and fixed on coverslips in 4% PFA for 30 minutes. Slides were then blocked in 5% BSA in PBS and incubated with primary antibodies overnight. Primary antibodies used were YAP (Cell Signaling Technology, 14074, 1:100), TAZ (Cell Signaling Technology, 72804s, 1:100), LCK (Santa Cruz, sc-71498, 1:100) and EPCAM (Abcam, ab71916, 1:100). Secondary antibodies used were Alexa Fluor anti-rabbit 594 (Invitrogen, A21442, 1:300), anti-mouse 488 (Invitrogen, A21202, 1:300) and anti-rabbit 488 (Invitrogen, A-11008, 1:300). Slides were mounted using Prolong Gold Anti-fade reagent (Thermo Fisher Scientific). Immunofluorescence was detected using confocal microscopy (Zeiss LSM 780, Zeiss) and a digital inverted microscope (Invitrogen EVOS M5000).

### Immunohistochemistry Assays

Immunohistochemistry assay was performed in paraffin-embedded tissue sections. Briefly, 5-μm sections were prepared following deparaffinization, hydration, antigen retrieval and stained with H&E or primary antibodies. Primary antibodies used were Ki67 (Novus Biological, NB110-89717H, 1:100), cleaved-caspase 3 (Cell signaling, 9661L, 1:400), CK19 (Cell signaling, 12434, 1:1200). For quantification of Ki67 or cleaved-caspase 3 positive cells, ten high-power fields (HPF) (20×) were used to manually count.

Quantitative Reverse Transcriptase Polymerase Chain Reaction (qRT-PCR)

Whole RNA was isolated from tissues or cultured cells using TRIzol (Invitrogen) based on standard procedure. Reverse transcription was performed using Moloney murine leukemia virus reverse transcriptase (Promega). q-PCR reaction was performed using SYBR green (Roche Diagnostics) on LightCycler 480 II (Roche Diagnostics). The ΔΔCt method was used to calculate the relative target gene expression, normalizing to 18S rRNA. The primers used for both human targets (in CAPITAL letters) and murine targets (lower case letters) are as shown in Table 6.

TABLE 6
Primer sequences (5′ to 3′, forward; reverse).

| | | SEQ ID | | SEQ ID |
|---|---|---|---|---|
| target | forward primer | NO | reverse primer | NO |
| YAP1 | CCCTCGTTTTGCCATGAACC | 1 | TGCCGAAGCAGTTCTTGCTG | 2 |
| TAZ | AGCAGAATTTTTCCAAGCAG | 3 | CTCCTTTCTCTCTTCTTTTTCC | 4 |
| LCK | GGGATCCCAGGATCTCACAA | 5 | TGTGCAGAGCGATAACCAGG | 6 |
| CTGF | GCAGCGGAGAGTCCTTCCAG | 7 | GGGCCAAACGTGTCTTCCAG | 8 |
| CYR61 | GAGTGGGTCTGTGACGAGGAT | 9 | GGTTGTATAGGATGCGAGGCT | 10 |
| MCL1 | GAGGGCGACTTTTGGCTAC | 11 | GTACCCGTCCAGCTCCTCTT | 12 |
| BCL2L1 | CCTTGGATCCAGGAGAACGG | 13 | AAGAGTGAGCCCAGCAGAAC | 14 |
| BCL2 | GCCTTCTTTGAGTTCGGTGG | 15 | GAAATCAAACAGAGGCCGCA | 16 |
| Yap1 | CAATACGGAATATCAATCCCAG | 17 | GATCGGAACTATTGGTTGTC | 18 |
| Taz | CAGAAGGGAAAGTAAACATGAG | 19 | ATCATTCATTCCAACATGCC | 20 |
| Lck | CATTAACTATGGGACCTTCAC | 21 | TCCAGGTTCTGAATGACTTC | 22 |
| Mcl1 | TAAGGACGAAACGGGACTGG | 23 | AGTTTGGTGGCTGGAGCTTTA | 24 |
| Ctgf | GGGCCTCTTCTGCGATTTC | 25 | ATCCAGGCAAGTGCATTGGTA | 26 |
| Cyr61 | GAACCGCGAGTTCTTTTCAA | 27 | AGGACGCACTTCACAGATCC | 28 |
| 18S | CGCTTCCTTACCTGGTTGAT | 29 | GAGCGACCAAAGGAACCATA | 30 |

### Cell Survival

Cell survival studies were performed. Briefly, cell death was quantified after staining with Hoechst 33342 (Invitrogen) and propidium iodide (Sigma). Cell apoptosis were evaluated using Apo-ONE homogeneous caspase-3/7 Assay (Promega) based on manufacturer's instructions. For colony formation assay, cells were fixed with 4% PFA for 20 minutes and stained with 0.05% crystal violet for 15 minutes.

### PDX Generation

Human CCA tumors were implanted in male NOD/SCID mice, age 6-8 weeks. Specifically, NOD/SCID mice were implanted with human CCA tumors in the right flank under aseptic conditions. Two months after implantation, PDX tissues were dissected and cut into fragment cube approximately 4 mm in each dimension. Fragment cubes were then implanted into the right lobe of the liver parenchyma.

### In Vivo Studies

For the subcutaneous SB1 model, resuspended SB1 cells (1×10⁶ cells in 50% DMEM and 50% Matrigel, 100 μL total volume) were injected into the flank area of male C57BL/6 mice. For the orthotopic SB1 model, resuspended SB1 cells (0.5×10⁶ cells in DMEM containing 10% FBS, 20 μL total volume) were injected the lateral aspect of the medial lobe using 27-gauge needle. Treatment for SB1 model were initiated 21 days after the implantation. Mice bearing orthotopic SB1 tumors were treated with gemcitabine (15 mg/kg) and cisplatin (1 mg/kg) intraperitoneally every three days. Normal saline was used as vehicle control. tMNV loaded with 5 μM siYAP, siTAZ, and siLCK (or loaded with scrambled control non-targeting siRNA) were administered intravenously every two days (2×10¹² particles/mouse in 200 μL). Treatment for PDX model were initiated 28 days after the implantation. Mice bearing orthotopic PDX tumors were treated with gemcitabine (4 mg/kg) and cisplatin (1 mg/kg) intraperitoneally every three days. tMNV containing 5 μM siYAP, siTAZ, and siLCK (or loaded with scrambled control non-targeting siRNA) were administered intravenously every two days (2×10¹² particles/mouse in 200 μL). Body weight was measured for every 3 days. Animals were sacrificed until the endpoint were reached. Samples were weighed and collected for molecular studies.

### Bioinformatical Analysis

For analysis of YAP1, TAZ, and LCK mRNA expression in CCA and normal liver tissue, transcriptome data were extracted from publicly available TCGA and Gene Expression Omnibus (GEO) databases (GSE26566). The GEO database (GSE26566) was also utilized to analyze the Ctgf and Cyr61 mRNA expression in CCA and normal liver tissue. TCGA data were processed and analysed using GEPIA. GEO data were processed and analysed using R (4.0.3) software.

### Flowcytometry

Tumor were dissociated with gentleMACS™ Octo Dissociator (Miltenyi) based on the manufacturer's protocol. CD45⁺ cells were isolated using CD45 (TIL) mouse microbeads (Miltenyi) and stained with Fixable Viability Stain 510 (BD Horizon™) for 15 minutes. Anti-Fc blocking reagent (Miltenyi) were added for 10 minutes prior to surface staining. The following antibodies were used for flow cytometry staining: F4/80-PE (REA126, Miltenyi), CD11b-PE-Cy5 (M1/70, eBioscience), CD206-PE-Cy7 (C068C2, BioLegend®), CCR2-APC-Vio®770 (REA538, Miltenyi), PD-L1-BV421 (10F.9G2, BioLegend®), F4/80-PEVio®770 (REA126, Miltenyi), CD11c-APC (REA754, Miltenyi), Ly6G-PE (Rat 1A8, Miltenyi), Ly6C-APC-Vio®770 (REA796, Miltenyi) CD3-APC-Vio®770 (REA641, Miltenyi), CD8-BV421 (53-6.7, BD Horizon™), CD11a-PEVio®770 (REA880, Miltenyi), PD-1-PerCP-Vio®700 (REA802, Miltenyi), granzyme B-PE (REA226, Miltenyi), CD45.1-Vioblue (A20, Miltenyi), CD45.2-PE-Vio®770 (104-2, Miltenyi), NK1.1-APC (REA1162, Miltenyi), Clec4F (MAB2784, R&D System). Clec4F antibody was conjugated with AlexaFluor® 647 antibody labeling kit (Invitrogen®). Data were analyzed using FlowJo software (FlowJo, LLC).

Preparation of tMNVs

Milk-derived nanovesicles (MNVs) were prepared using fat-free milk as described elsewhere (Matsuda et al., Methods Mol. Biol., 1740:187-197 (2018)). Concentration and size of isolated MNVs were assessed by a NanoSight instrument (Malvern Panalytical). 3WJ RNA was synthesized from three component strands. Cholesterol-triethylene-glycol (TEG) was conjugated in the a3 WJ strand. EpCAM RNA apt sequence was incorporated in the b3WJ strand. Alexa647 was incorporated in the c3WJ strand. For combining 3WJ RNA with MNVs, an equal volume of 3WJ RNA and MNVs were gently mixed and incubated for 2 hours at room temperature for further studies.

### Biodistribution Analysis

NOD/SCID mice bearing orthotopic PDX tumor or C57BL/6 mice bearing orthotopic SB1 tumor were administered tMNVs (2×10¹² particles/mouse in 200 μL). 6 hours after the tail vein injection of tMNVs, fresh samples (subcutaneous tumor, orthotopic tumor, paired liver, lung, heart, spleen, kidney) were collected immediately and embedded in OCT, and then frozen sectioning were performed. Alexa647 positive cells were quantified in 10 high powered fields using a digital inverted microscope (Invitrogen EVOS M5000).

tMNVs Uptake by Murine CCA Tissues

Three weeks following SB1 cells orthotopic implantation, fresh SB1 tumor and normal adjacent liver were collected and cut into 3×3×3 mm cubes and placed in 96 well plates. Triplicate samples were pooled to make single-cell suspensions. Samples were incubated for 24 hours with 2×10¹² nanovesicles with Alexa647 labeled aptamer and stained for flow cytometry.

tMNVs Uptake by CCA Cells

HuCCT1 and SB1 cells were plated in 35 mm Glass Bottom Dishes (MatTek) and treated with tMNVs (1×10¹¹ particles). For labeling tMNVs, PKH67 (Thermo Fisher) was added to the pellet, which was re-suspended in PBS and ultracentrifuged for 90 minutes at 100,000×g at 4° C. twice. The obtained pellet was sterile filtered through 0.22 μm syringe filter and re-suspended in DMEM containing 10% FBS. Cells were incubated with tMNVs for 12 hours and fluorescence was detected using a digital inverted microscope (Invitrogen EVOS M5000).

### Statistical Analysis

Statistical analysis was performed using GraphPad Prism 8 software (GraphPad software). Data were presented as mean±standard deviation (SD). Comparisons were done between two experimental groups using unpaired two-tailed Student's t-test. Differences between multiple groups were compared using one-way ANOVA test. Differences were considered significant when p values were <0.05 (*), <0.01 (**), <0.001 (***), or <0.0001 (****).

### Example 3: Cholangiocarcinoma and EpCAM binding aptamers

This Example describes the design and characterization of CCA binding aptamers. Cholangiocarcinoma aptamers were identified from an aptamer pool by Sequential Evolution of Ligands by Exponential Enrichment (SELEX). A normal human hepatocyte cell line was utilized as a negative screen and HuCCT1 cholangiocarcinoma cells utilized for positive selection. Repeated rounds of incubation with the cell lines and then PCR amplification were performed. Deep sequencing was performed after amplification and enrichment was noted.

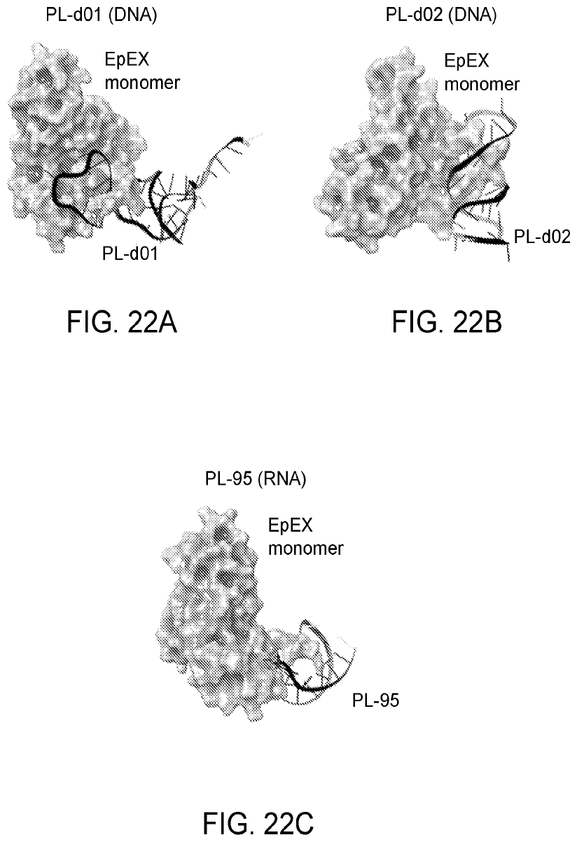

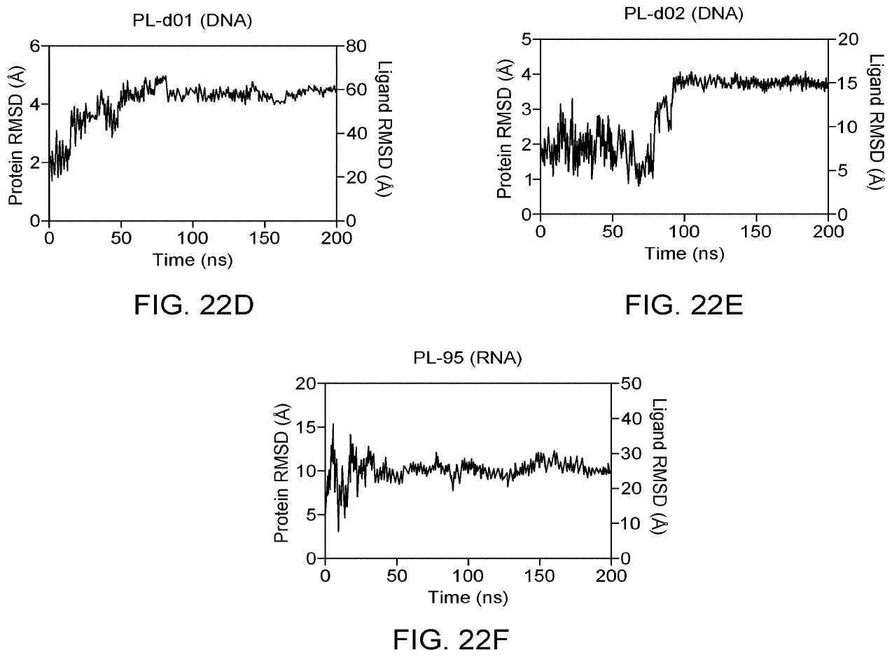

The 3D crystal structure of EpCAM extracellular domain (EpEX) was obtained from the RCSB protein data bank (PDB ID: 6107), and the complexed structure was subsequently cropped using the Schrödinger Maestro software to eliminate the ScFv structure (Schrödinger Release 2023-3, Maestro, Schrödinger, LLC, New York, NY). The 3D folded structures of the DNA and RNA aptamers (FIGS. 22A-22C) were generated using the 3dRNA/DNA server (Wang et al., Int. J. Mol. Sci., 20 (17): 4116 (2019)). The top predicted structure of each aptamer was selected for molecular dynamics studies.

Protein-protein docking was performed with the prepared structures as follows: standard mode, 70,000 rotations, 30 poses (FIGS. 22D-22F). The docked poses with the lowest PIPER scores were selected for molecular dynamics (MD). A system was built with TIP3P solvent, 0.15 M NaCl and Na⁻ ions were added to neutralize the system. The system had a buffer space of 10 Å (10×10×10). Two hundred nanosecond MD simulations of the EpEX-aptamer docked poses were performed under normal temperature and pressure conditions in Schrodinger Desmond (Schrödinger Release 2023-3). At the completion of MD, simulations interactions diagrams were generated, and the raw data was extracted. The root mean square deviation (RMSD) plots were constructed to evaluate the stability of the EpEX-aptamer complexes. The root mean square fluctuation (RMSF) data was used to identify the aptamer-contacted residues.

The aptamers that met the following criteria were selected for in vitro validation: (1) short sequences (x<50 nucleotides), (2) formed the classical loop stem 3D structure, (3) terminal nucleotides do not interact with EpEX, (4) EpEX monomer-aptamer complex RMSD within 4 Å throughout the MD simulation. Based on this criteria, two DNA aptamers, PL-d01 and PL-d02, as well as one RNA aptamer, PL-95 were selected for validation.

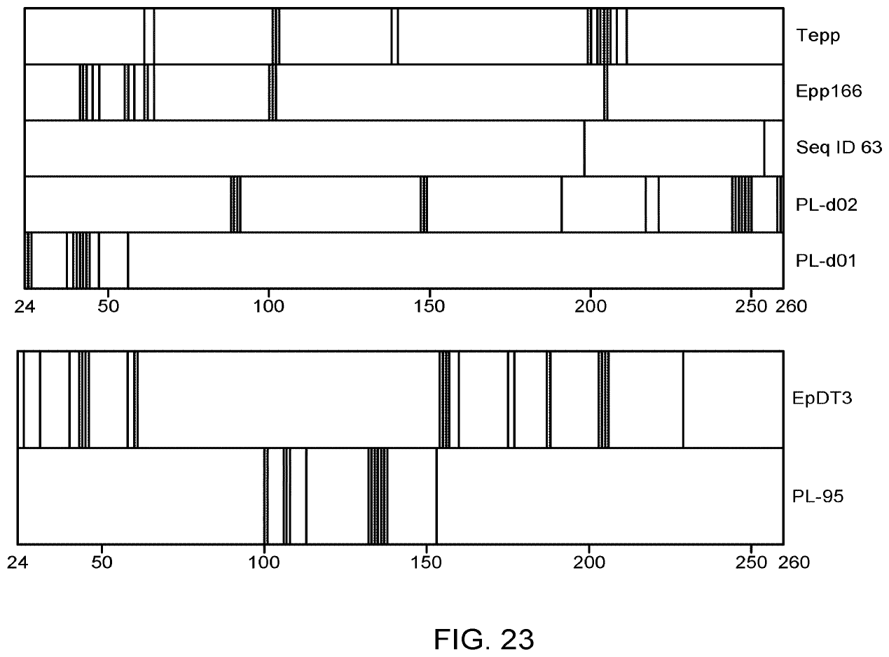

To identify binding residues, docking and molecular dynamics simulations were performed with EpEX and other DNA aptamers (JYK-01, Ep166, tepp, SYL3C, and SEQ ID NO: 63 (Asic) set forth in U.S. Patent Application Publication No. 2022/0340906, or RNA aptamers (EpDT3 and Ep23) reported to target EpCAM. The EpCAM residues that were in contact with the aptamers PL-d01, PL-d02 and PL-95 or with other DNA aptamers (Asic, Epp166, Tepp) and RNA aptamers (EpDT3) were identified from molecular dynamics simulations of aptamer binding with the EpCAM extracellular residues performed using Schrodinger Maestro software. The regions on EpCAM where the aptamers interacted during the molecular dynamics simulations are indicated for each aptamer (FIG. 23).

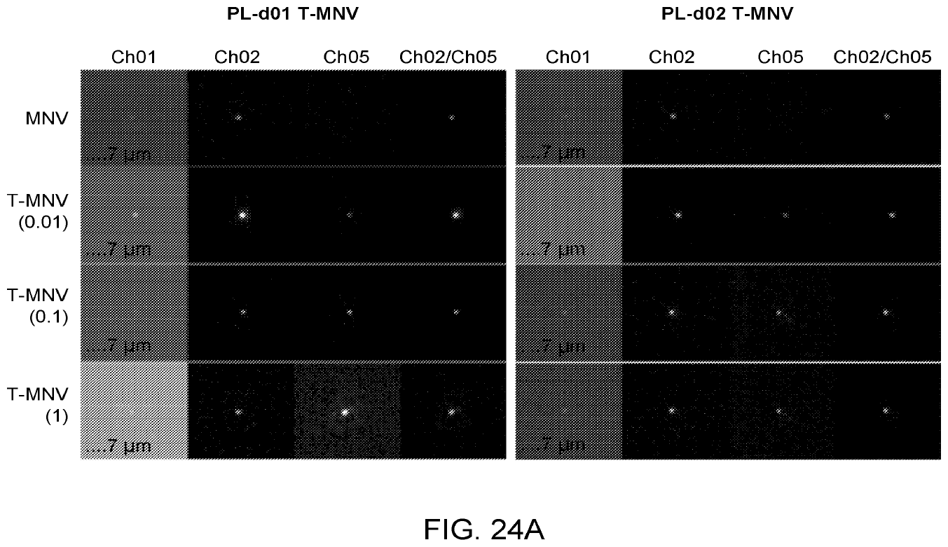

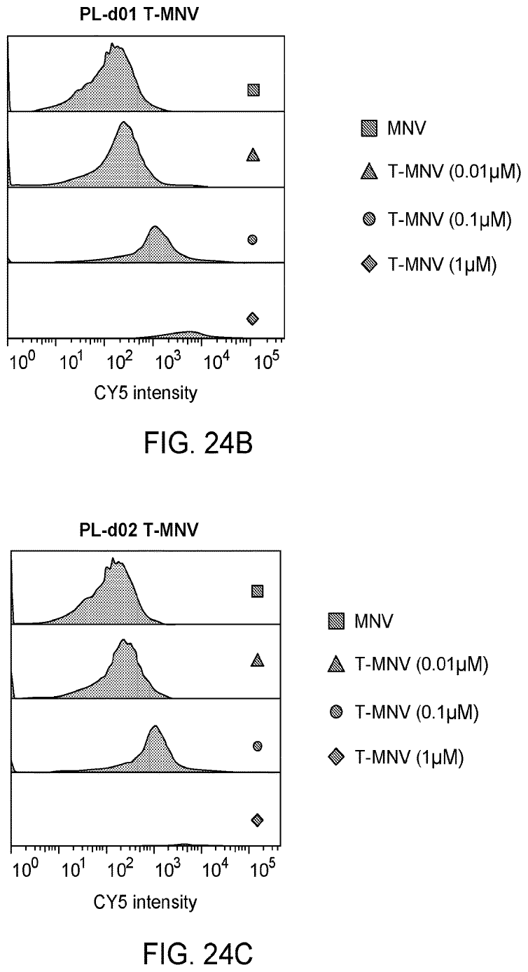

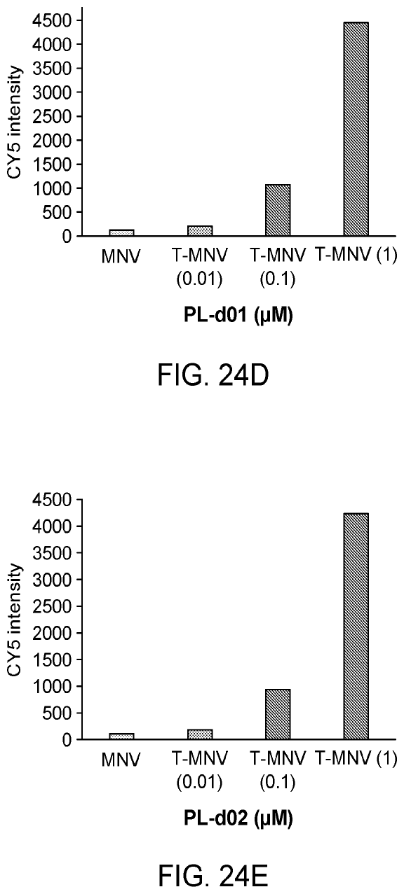

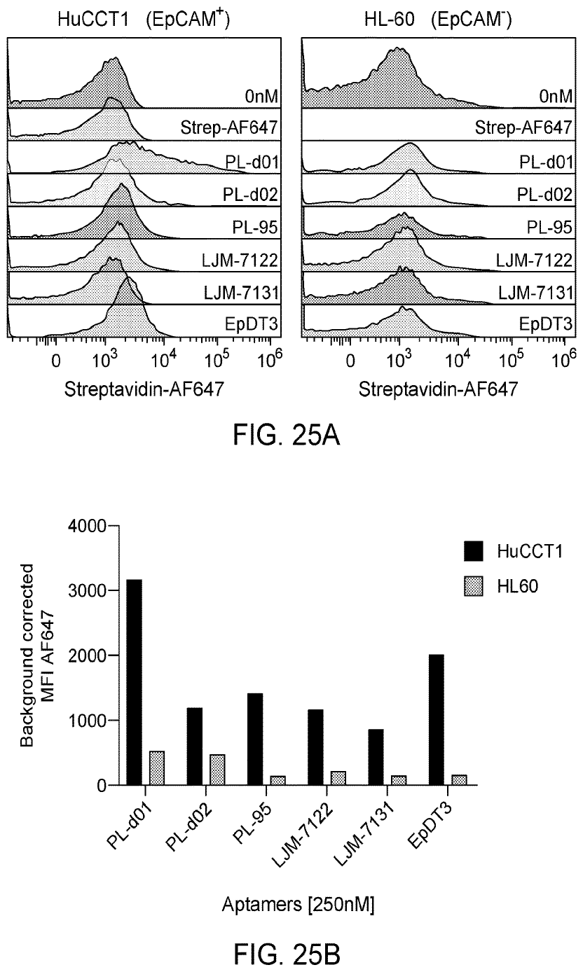

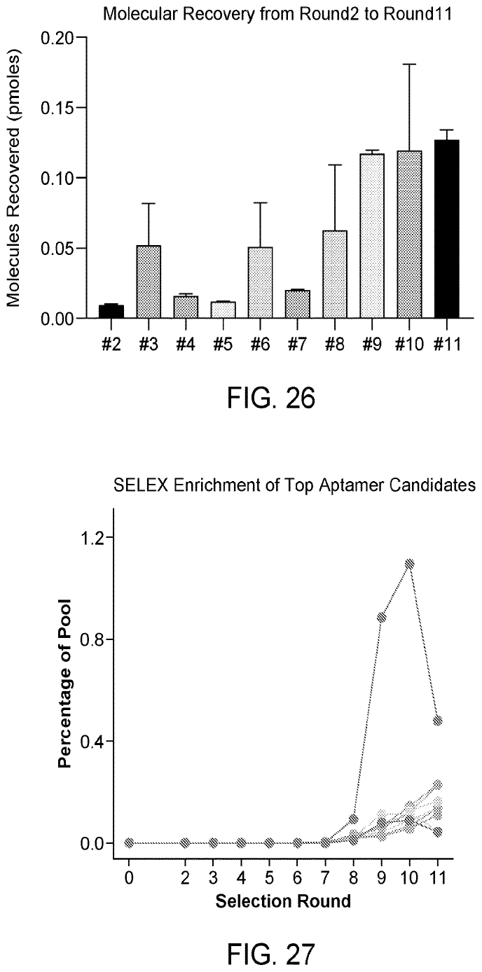

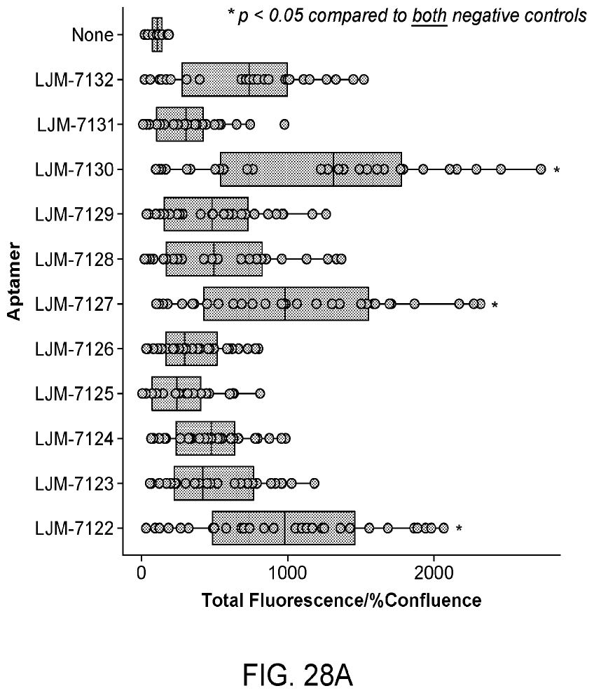

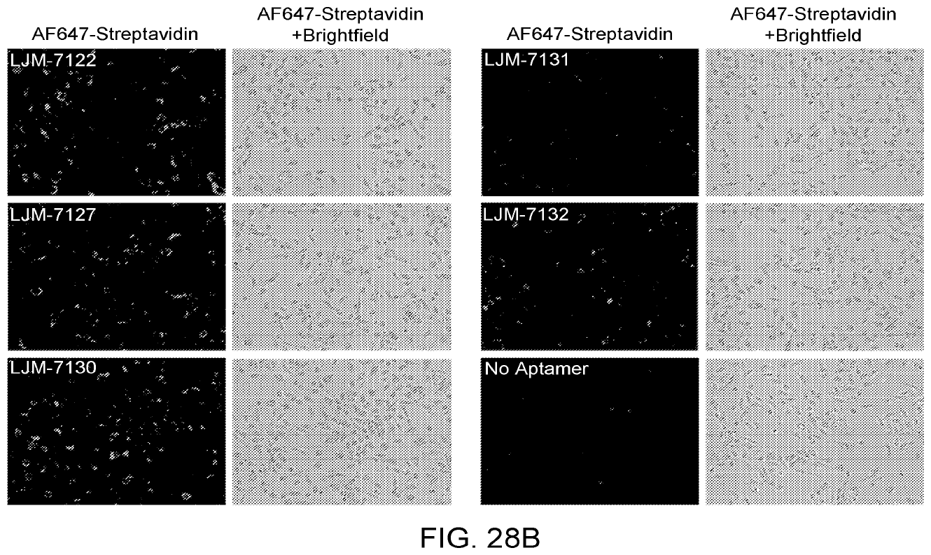

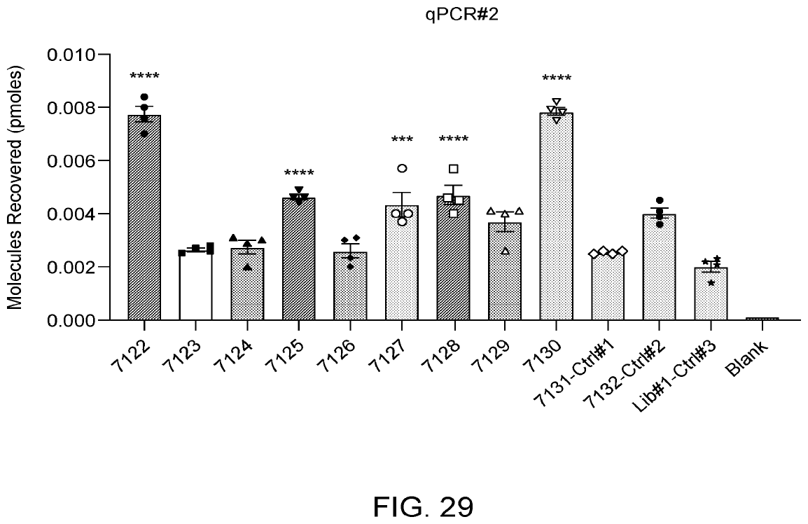

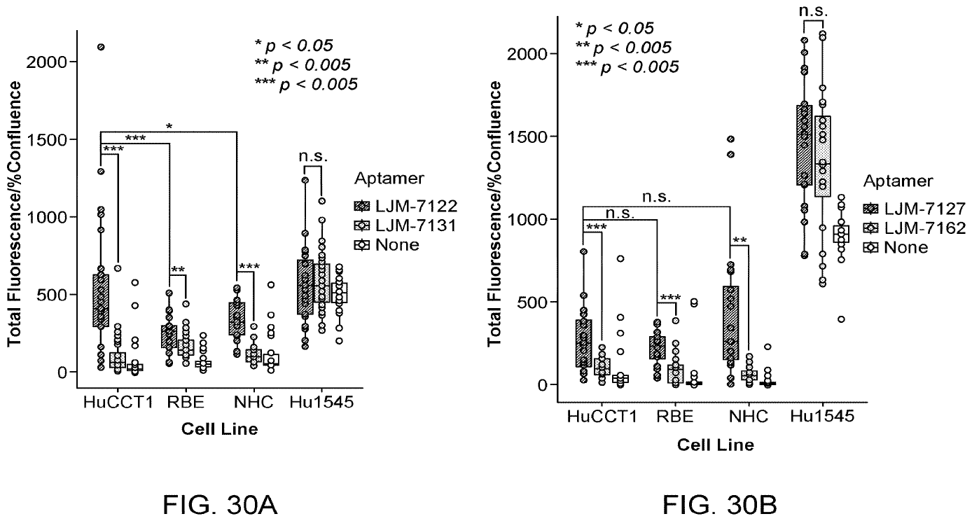

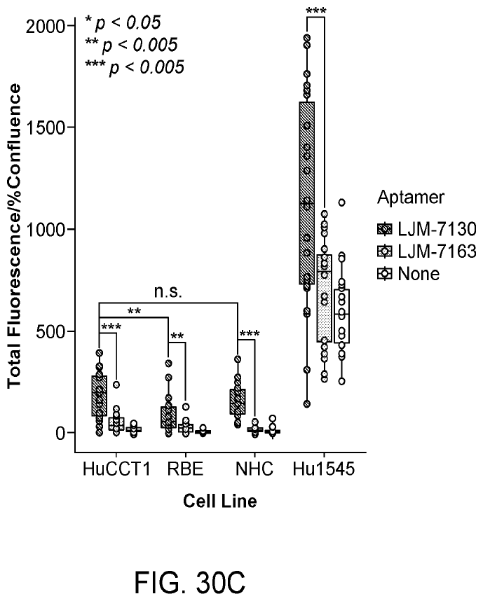

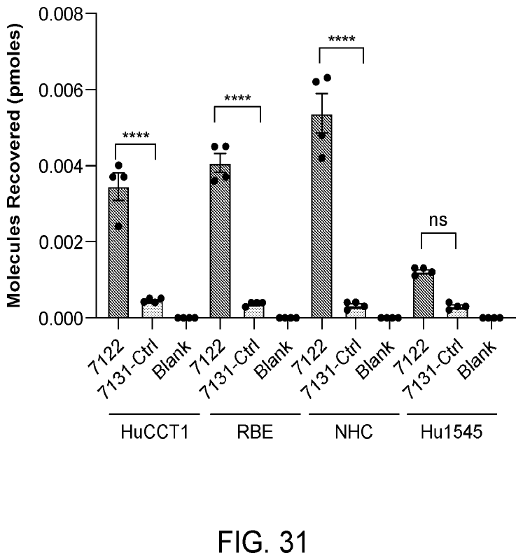

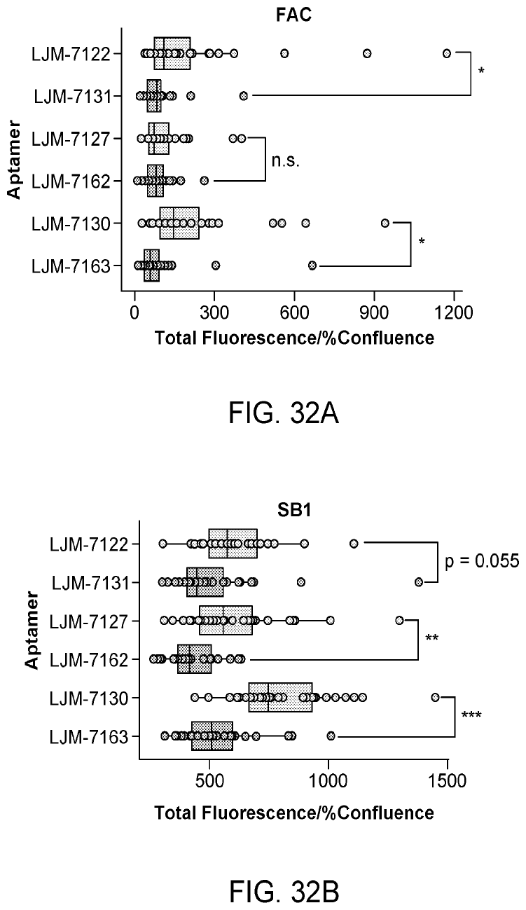

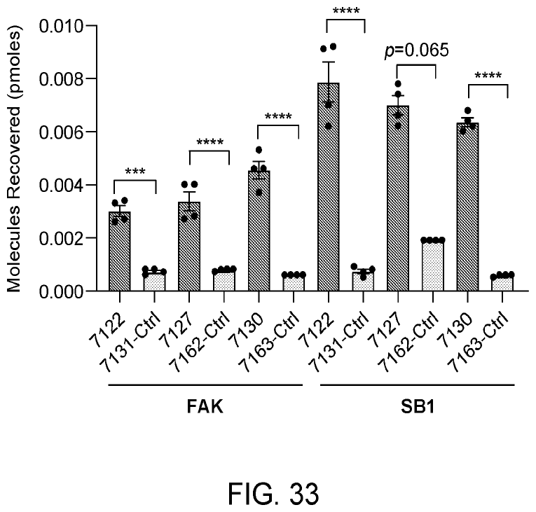

The ability of the DNA aptamers to be incorporated onto the surface of MNVs was determined using tMNVS generated from MNV labeled with 2.00×10¹¹ DiO (detected in green channel Ch02) and decorated with 0.01, 0.1, or 1 μM of CY5-PLd01-TEGCholesterol or CY5-PLd02-TEGCholesterol (detected in red channel Ch05). ImageStream flow cytometer was used to detect aptamer labels on MNVs (FIG. 24A). Cy5 fluorescence intensity plots are shown for different concentrations of aptamer PLd01 (FIG. 24B) or PLd02 (FIG. 24C), with quantitative data for PLd01 (FIG. 24D) and PLd02 (FIG. 24E) demonstrating the ability of these DNA aptamers to decorate and bind to MNV in a concentration-dependent manner.

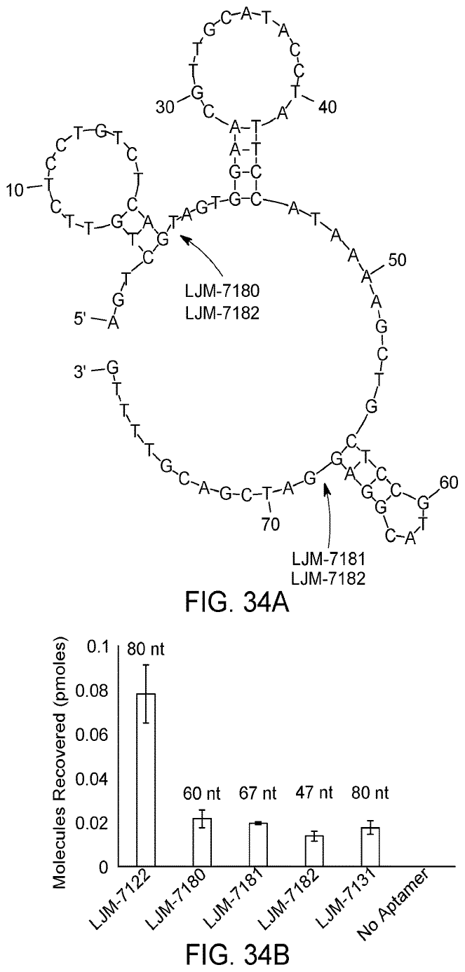

The secondary structure of the aptamer candidate LJM-7122 was predicted by mfold software (FIG. 34A). Truncations of the lead candidate producing additional aptamer candidates were produced and the cholangiocarcinoma cell binding activity was evaluated by quantitative PCR (FIG. 34B). This demonstrated that truncations of the lead candidate had reduced cell binding activity.

TABLE 7
Exemplary oligonucleotide sequences.

| Oligo- | | |
|---|---|---|
| nucleotide | | SEQ |
| ID | Sequence | ID NO: |
| LJM-7122 | /56-FAM/AGTCTGTTCTCCTGTCTCAGTAGTGGAACGTTGCATACCTATTCCATAAAAGCTGCTCCGTACGGAGG | 31 |
| | ATCGACGTTTTG/3BioTEG/ | |
| LJM-7123 | /56-FAM/AGTCTGTTCTCCTGTCTCAGGTACTGTGACGCGTTATTTCGGGCAGAATAAGGCTGTGCATACGGAGG | 32 |
| | ATCGACGTTTTG/3BioTEG/ | |
| LJM-7124 | /56-FAM/AGTCTGTTCTCCTGTCTCAGATCTGGCTAGAGCGCATAGAGACGTGCAAAGCACTGTGAATACGGAGG | 33 |
| | ATCGACGTTTTG/3BioTEG/ | |
| LJM-7125 | /56-FAM/AGTCTGTTCTCCTGTCTCAGCTGAGACGCGCACGAATGATTTACAGCAGTAAGCCGTGAATACGGAGG | 34 |
| | ATCGACGTTTTG/3BioTEG/ | |
| LJM-7126 | /56-FAM/AGTCTGTTCTCCTGTCTCAGGACGTGGGGCTACGCCTAGCAACACTGTAAACGGTAAGGTTACGGAGG | 35 |
| | ATCGACGTTTTG/3BioTEG/ | |
| LJM-7127 | /56-FAM/AGTCTGTTCTCCTGTCTCAGATTGGAACGATCCATCTCTAGATTCAGCCAGGTCCGCGCATACGGAGG | 36 |
| | AATCGCGTTTTG/3BioTEG/ | |
| LJM-7128 | /56-FAM/AGTCTGTTCTCCTGTCTCAGCCGAGACGCGCATTTGCGCTGTAAAATACCACATACCTTTTACGGAGG | 37 |
| | ATCGACGTTTTG/3BioTEG/ | |
| LJM-7129 | /56-FAM/AGTCTGTTCTCCTGTCTCAGATGAGACGTCCGTAGGACTGTAACTCAGAGCCCTCGAGTGTACGGAGG | 38 |
| | ATCGACGTTTTG/3BioTEG/ | |
| LJM-7130 | /56-FAM/AGTCTGTTCTCCTGTCTCAGAAACTGGAACGTTCTATTTCACAGCGGCTCGCCTGGTCCATACGGAGG | 39 |
| | ATCGACGTTTTG/3BioTEG/ | |
| LJM-7131 | /56-FAM/AGTCTGTTCTCCTGTCTCAGTCAGAGATAGAGTTCAAGTTCAGTACCCCGTTTTCGCAACTACGGAGG | 40 |
| | ATCGACGTTTTG/3BioTEG/ | |
| LJM-7132 | /56-FAM/AGTCTGTTCTCCTGTCTCAGCTGAAAGGGCCGACTGGATTATGCATCTGTCGAATGTGGTTACGGAGG | 41 |
| | ATCGACGTTTTG/3BioTEG/ | |
| LJM-7162 | /56-FAM/AGTCTGTTCTCCTGTCTCAGCGCCGTGTAACCCTGCGAACATATTTCCTGATGAGAAGCCTACGGAGG | 42 |
| | ATCGACGTTTTG/3BioTEG/ | |
| LJM-7162 | /56-FAM/AGTCTGTTCTCCTGTCTCAGATTACGAACGCACTCTGTCGTCCATCCCTTGGAGGAAGTCTACGGAGG | 43 |
| | ATCGACGTTTTG/3BioTEG/ | |
| LJM-7180 | TAGTGGAACGTTGCATACCTATTCCATAAAAGCTGCTCCGTACGGAGGATCGACGTTTTG | 44 |
| LJM-7181 | AGTCTGTTCTCCTGTCTCAGTAGTGGAACGTTGCATACCTATTCCATAAAAGCTGCTCCGTACGGAG | 45 |
| LJM-7182 | TAGTGGAACGTTGCATACCTATTCCATAAAAGCTGCTCCGTACGGAG | 46 |

/56-FAM/ indicates 5′ fluorescein modification
/3BioTEG/ indicates 3′ triethylene glycol biotin modification

### Example 4: EpCAM Binding Aptamers and Cancer

This Example describes using EpCAM binding aptamers to treat cancer.

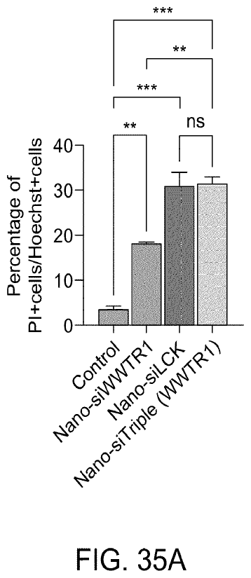

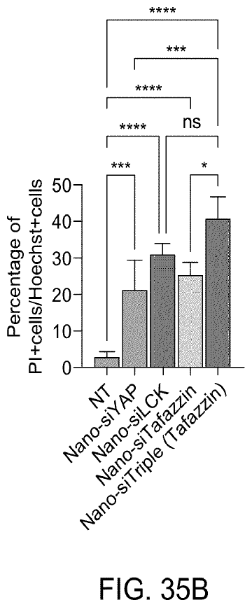

Murine cholangiocarcinoma cells (SB1) displayed increased cell death when exposed to EPCAM aptamer decorated milk-derived nanovesicles loaded with siRNA for WWTR1, LCK, a triple target (WWTR1, YAP, LCK) (FIG. 35A) for 48 hours. Similarly, SB1 cells displayed increased cell death at 48 hours when exposed to EPCAM aptamer decorated milk-derived nanovesicles loaded with siRNA for YAP, loaded with siRNA for LCK, loaded with siRNA for tafazzin, or triple loaded with siRNA for YAP, siRNA for LCK, and siRNA for tafazzin.

Together, these results demonstrate that inhibiting expression of one or more of a YAP polypeptide, a TAZ polypeptide, a tafazzin polypeptide, or a LCK polypeptide can increase cell death of cancer cells such as CCA cells.

TABLE 8
Sequences of exemplary oligonucleotides.

| Oligo- | | |
|---|---|---|
| nucleotide | | SEQ |
| ID | Sequence | ID NO: |
| PL-d01 | Biotin-C*A*C*ACTTCTAAGAGGG*AACAGACGTCGATCTTTGCGGAACT*CCTG*C*G* | 47 |
| PL-d02 | Biotin-G*A*C*CCACGGAGGTTG*CGTCCGTG*GGGG*A*C* | 48 |
| PL-95 | Biotin-AUAGUCGCGACUGGUCGACUA | 49 |
| EpDT3 | Biotin-GCGACUGGUUACCCGGUCG | 50 |
| Cy5-PLd01- | Cy5-C*A*C*ACTTCTAAGAGGG*AACAGACGTCGATCTTTGCGGAACT*CCTG*C*G*-TEGChol | 51 |
| TEGChol | | |
| Cy5-PLd02- | Cy5-G*A*C*CCACGGAGGTTG*CGTCCGTG*GGGG*A*C*-TEGChol | 52 |
| TEGChol | | |

The following modifications were incorporated into some of the sequences: 5′ biotin tag, (*) phosphorothioate modified bases, 5′ Cy5 tag, and 3′ triethylene glycol cholesteryl tag.

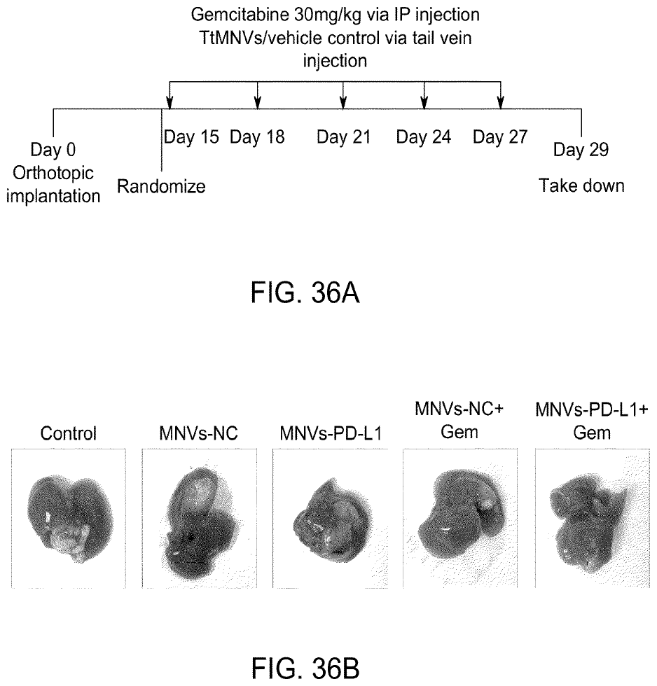

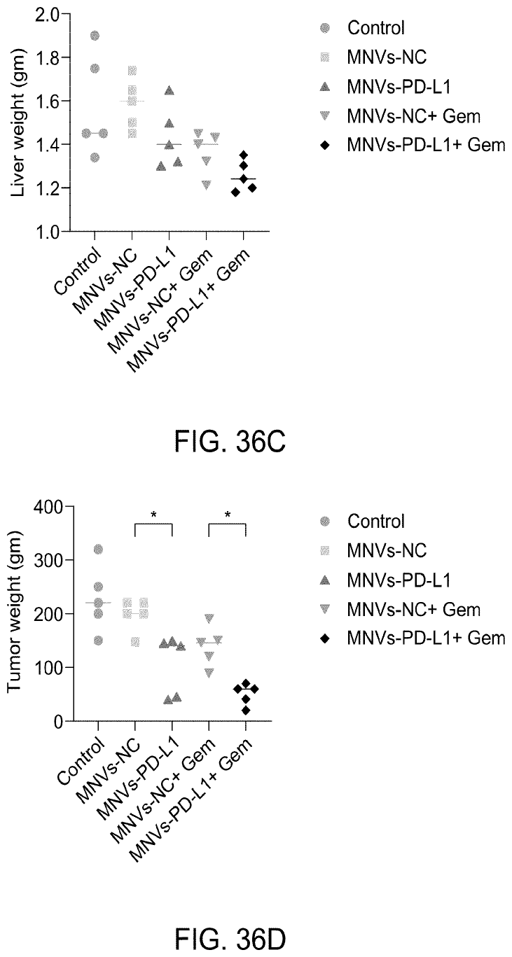

Animals (6 in each group) received vehicle control, EpCAM targeted tMNV loaded with non-targeting control siRNA (NC), or EpCAM targeted tMNV loaded with siRNA to PD-L1, along with gemcitabine. tMNV and gemcitabine were administered every three days for five doses, starting fifteen days after orthotopic implantation of SB1 cells. A schematic of the treatment protocol is shown in FIG. 36A. Liver weight (FIG. 36C) and tumor weight (FIG. 36D) were assessed on day 29 for all groups.

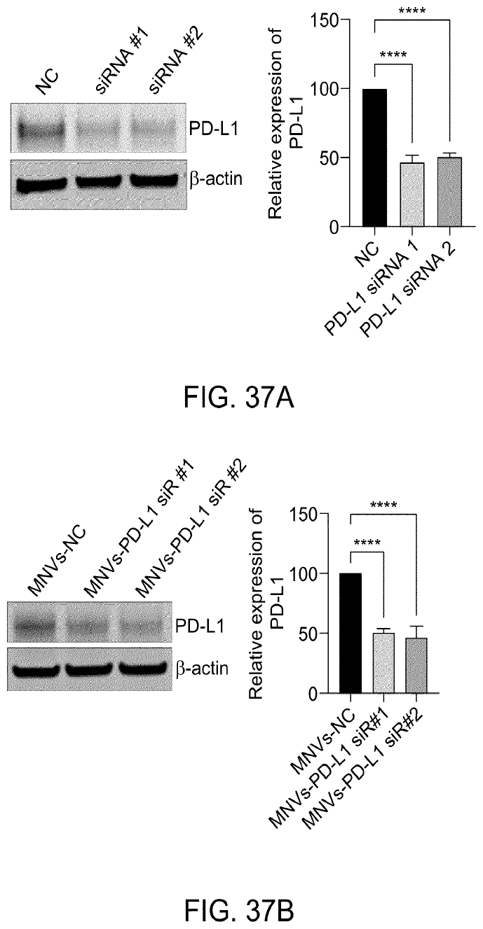

Silencing efficiency of siRNA to PD-L1 in HuCCT1 cells by transfection using lipofectamine 2000 (FIG. 37A) was compared with that of siRNA to PD-L1 delivered using tMNV loaded with siRNA to PD-L1 (FIG. 37B). Data are shown for two different siRNA with immunoblots showing expression of PD-L1 protein expression and quantitative data (mean and SD) from three separate determinations, * p<0.0001.

### Example 5: Treating Cancer

A human identified as having cancer (e.g., cholangiocarcinoma) is administered a composition including one or more vesicles (e.g., nanovesicles) where vesicles in the composition have: (a) a first inhibitor located within the vesicle, a second inhibitor located within the vesicle, and a third inhibitor located within the vesicle, where each inhibitor is selected from the group consisting of an inhibitor of a YAP polypeptide, an inhibitor of a TAZ polypeptide, an inhibitor of a LCK polypeptide, and an inhibitor of a TRAIL polypeptide, and (b) a targeting moiety (e.g., an EpCAM aptamer) located on a surface of the vesicle, where the targeting moiety binds to a molecule present on the surface of a cancer cell within the cancer. The administered vesicle(s) can reduce the number of cancer cells present within the human.

### Example 6: Treating Cancer

A human identified as having cancer (e.g., cholangiocarcinoma) is administered a composition including one or more vesicles (e.g., nanovesicles) where vesicles in the composition have: (a) an inhibitor of a LCK polypeptide and/or an inhibitor of a TRAIL polypeptide located within the vesicle, and (b) a targeting moiety (e.g., an EpCAM aptamer) located on a surface of the vesicle, where the targeting moiety binds to a molecule present on the surface of a cancer cell within the cancer. The administered vesicle(s) can reduce the number of cancer cells present within the human.

### Example 7: Exemplary Embodiments

Embodiment 1. A vesicle comprising a first inhibitor located within said vesicle, a second inhibitor located within said vesicle, a third inhibitor located within said vesicle, and a targeting moiety located on a surface of said vesicle, wherein said first inhibitor inhibits expression or activity of a first polypeptide, wherein said second inhibitor inhibits expression or activity of a second polypeptide, wherein said third inhibitor inhibits expression or activity of a third polypeptide, wherein said first polypeptide, said second polypeptide, and said third polypeptide are selected from the group consisting of a YAP polypeptide, a TAZ polypeptide, a LCK polypeptide, and a TRAIL polypeptide, wherein said targeting moiety binds to a molecule present on the surface of a cancer cell, and wherein said vesicle has a longest diameter of 5 nm to 500 nm.
Embodiment 2. The vesicle of embodiment 1, wherein said first polypeptide is said YAP polypeptide, wherein said second polypeptide is said TAZ polypeptide, and wherein said third polypeptide is said LCK polypeptide.
Embodiment 3. The vesicle of embodiment 1, wherein said first polypeptide is said YAP polypeptide, wherein said second polypeptide is said TAZ polypeptide, and wherein said third polypeptide is said TRAIL polypeptide.
Embodiment 4. The vesicle of embodiment 1, wherein said first polypeptide is said YAP polypeptide, wherein said second polypeptide is said LCK polypeptide, and wherein said third polypeptide is said TRAIL polypeptide.
Embodiment 5. The vesicle of embodiment 1, wherein said first polypeptide is said TAZ polypeptide, wherein said second polypeptide is said LCK polypeptide, and wherein said third polypeptide is said TRAIL polypeptide.
Embodiment 6. The vesicle of embodiment 1, wherein one of said first polypeptide, said second polypeptide, and said third polypeptide is said YAP polypeptide, and wherein one of said first inhibitor, said second inhibitor, and said third inhibitor inhibits expression or activity of said YAP polypeptide.
Embodiment 7. The vesicle of embodiment 6, wherein said one of said first inhibitor, said second inhibitor, and said third inhibitor is an siRNA molecule that inhibits expression of said YAP polypeptide.
Embodiment 8. The vesicle of embodiment 6, wherein said one of said first inhibitor, said second inhibitor, and said third inhibitor inhibits activity of said YAP polypeptide.
Embodiment 9. The vesicle of embodiment 8, wherein said one of said first inhibitor, said second inhibitor, and said third inhibitor is selected from the group consisting of CA3 and verteporfin.
Embodiment 10. The vesicle of embodiment 1, wherein one of said first polypeptide, said second polypeptide, and said third polypeptide is said TAZ polypeptide, and wherein one of said first inhibitor, said second inhibitor, and said third inhibitor inhibits expression or activity of said TAZ polypeptide.
Embodiment 11. The vesicle of embodiment 10, wherein said one of said first inhibitor, said second inhibitor, and said third inhibitor is an siRNA molecule that inhibits expression of said TAZ polypeptide.
Embodiment 12. The vesicle of embodiment 10, wherein said one of said first inhibitor, said second inhibitor, and said third inhibitor inhibits activity of said TAZ polypeptide.
Embodiment 13. The vesicle of embodiment 1, wherein one of said first polypeptide, said second polypeptide, and said third polypeptide is said LCK polypeptide, and wherein one of said first inhibitor, said second inhibitor, and said third inhibitor inhibits expression or activity of said LCK polypeptide.
Embodiment 14. The vesicle of embodiment 13, wherein said one of said first inhibitor, said second inhibitor, and said third inhibitor is an siRNA molecule that inhibits expression of said LCK polypeptide.
Embodiment 15. The vesicle of embodiment 13, wherein said one of said first inhibitor, said second inhibitor, and said third inhibitor inhibits activity of said LCK polypeptide.
Embodiment 16. The vesicle of embodiment 15, wherein said one of said first inhibitor, said second inhibitor, and said third inhibitor is selected from the group consisting of dasatinib and saracatinib.
Embodiment 17. The vesicle of embodiment 1, wherein one of said first polypeptide, said second polypeptide, and said third polypeptide is said TRAIL polypeptide, and wherein one of said first inhibitor, said second inhibitor, and said third inhibitor inhibits expression or activity of said TRAIL polypeptide.
Embodiment 18. The vesicle of embodiment 17, wherein said one of said first inhibitor, said second inhibitor, and said third inhibitor is an siRNA molecule that inhibits expression of said TRAIL polypeptide.
Embodiment 19. The vesicle of embodiment 17, wherein said one of said first inhibitor, said second inhibitor, and said third inhibitor inhibits activity of said TRAIL polypeptide.
Embodiment 20. The vesicle of any one of embodiments 1-20, wherein said vesicle comprises a fourth inhibitor located within said vesicle, wherein said fourth inhibitor inhibits expression or activity of a fourth polypeptide.
Embodiment 21. The vesicle of embodiment 20, wherein said first inhibitor inhibits expression or activity of said YAP polypeptide, wherein said second inhibitor inhibits expression or activity of said TAZ polypeptide, wherein said third inhibitor inhibits expression or activity of said LCK polypeptide, and wherein said fourth inhibits expression or activity of said TRAIL polypeptide.
Embodiment 22. The vesicle of any one of embodiments 1-21, wherein said vesicle comprises an imaging agent.
Embodiment 23. The vesicle of embodiment 22, wherein said imaging agent is selected from the group consisting of a fluorophore, a fluorescent dye, and a radionucleotide.
Embodiment 24. The vesicle of any one of embodiments 1-23, wherein said cancer cell is located within a liver, gall bladder, small intestine, or bile duct of a mammal.
Embodiment 25. The vesicle of embodiment 24, wherein said mammal is a human.
Embodiment 26. The vesicle of any one of embodiments 1-25, wherein said cancer cell is a liver cancer cell, a gall bladder cancer cell, a small intestine cancer cell, or a bile duct cancer cell.
Embodiment 27. The vesicle of any one of embodiments 1-25, wherein said cancer cell is a cholangiocarcinoma cancer cell.
Embodiment 28. The vesicle of any one of embodiments 1-27, wherein said molecule is an epithelial cell adhesion molecule (EpCAM) polypeptide, an EPH receptor A2 (EPHA2) polypeptide, an AXL polypeptide, a G Protein-coupled receptor class C group 5 member A (GPRC5a) m polypeptide, a GPRC5c polypeptide, an epidermal growth factor receptor (EGFR) polypeptide, a cytokeratin-19 (CK19) polypeptide, an osteopontin (SPP1) polypeptide, or a Sox9 polypeptide.
Embodiment 29. The vesicle of any one of embodiments 1-28, wherein said targeting moiety is an antibody.
Embodiment 30. The vesicle of any one of embodiments 1-28, wherein said targeting moiety is a nucleic acid aptamer.
Embodiment 31. The vesicle of embodiment 32, wherein said nucleic acid aptamer is an EpCAM aptamer, an AXL aptamer, an EGFR aptamer, a CK19 aptamer, a SPP1 aptamer, or a Sox9 aptamer.
Embodiment 32. The vesicle of any one of embodiments 30-31, wherein said nucleic acid aptamer is an RNA aptamer.
Embodiment 33. The vesicle of any one of embodiments 30-31, wherein said nucleic acid aptamer is a DNA aptamer.
Embodiment 34. The vesicle of any one of embodiments 1-33, wherein said longest diameter is 100 nm to 500 nm.
Embodiment 35. The vesicle of any one of embodiments 1-33, wherein said longest diameter is 200 nm to 400 nm.
Embodiment 36. A vesicle comprising an inhibitor located within said vesicle and a targeting moiety located on a surface of said vesicle, wherein said inhibitor inhibits expression or activity of a LCK polypeptide or a TRAIL polypeptide, wherein said targeting moiety binds to a molecule present on the surface of a cancer cell, and wherein said vesicle has a longest diameter of 5 nm to 500 nm.
Embodiment 37. The vesicle of embodiment 36, wherein said inhibitor inhibits expression or activity of said LCK polypeptide.
Embodiment 38. The vesicle of embodiment 37, wherein said inhibitor is an siRNA molecule that inhibits expression of said LCK polypeptide.
Embodiment 39. The vesicle of embodiment 37, wherein said inhibitor inhibits activity of said LCK polypeptide.
Embodiment 40. The vesicle of embodiment 39, wherein said inhibitor is selected from the group consisting of dasatinib and saracatinib.
Embodiment 41. The vesicle of any one of embodiments 36-40, wherein said vesicle comprises a second inhibitor located within said vesicle.
Embodiment 42. The vesicle of embodiment 41, wherein said second inhibitor inhibits expression or activity of a YAP polypeptide, a TAZ polypeptide, or said TRAIL polypeptide.
Embodiment 43. The vesicle of embodiment 42, wherein said second inhibitor inhibits expression or activity of said YAP polypeptide.
Embodiment 44. The vesicle of embodiment 42, wherein said second inhibitor inhibits expression or activity of said TAZ polypeptide.
Embodiment 45. The vesicle of embodiment 42, wherein said second inhibitor inhibits expression or activity of said TRAIL polypeptide.
Embodiment 46. The vesicle of any one of embodiments 6-40, wherein said vesicle comprises a second inhibitor located within said vesicle and a third inhibitor located within said vesicle.
Embodiment 47. The vesicle of embodiment 46, wherein said second inhibitor and said third inhibitor inhibits expression or activity of a YAP polypeptide, a TAZ polypeptide, or said TRAIL polypeptide.
Embodiment 48. The vesicle of any one of embodiments 36-40, wherein said vesicle comprises a second inhibitor located within said vesicle, a third inhibitor located within said vesicle, and a fourth inhibitor located within said vesicle.
Embodiment 49. The vesicle of embodiment 48, wherein said second inhibitor inhibits expression or activity of a YAP polypeptide, wherein said third inhibitor inhibits expression or activity of a TAZ polypeptide, and wherein said fourth inhibitor inhibits expression or activity of said TRAIL polypeptide.
Embodiment 50. The vesicle of embodiment 36, wherein said inhibitor inhibits expression or activity of said TRAIL polypeptide.
Embodiment 51. The vesicle of embodiment 50, wherein said inhibitor is an siRNA molecule that inhibits expression of said TRAIL polypeptide.
Embodiment 52. The vesicle of embodiment 50, wherein said inhibitor inhibits activity of said TRAIL polypeptide.
Embodiment 53. The vesicle of any one of embodiments 50-52, wherein said vesicle comprises a second inhibitor located within said vesicle.
Embodiment 54. The vesicle of embodiment 53, wherein said second inhibitor inhibits expression or activity of a YAP polypeptide, a TAZ polypeptide, or said LCK polypeptide.
Embodiment 55. The vesicle of embodiment 54, wherein said second inhibitor inhibits expression or activity of said YAP polypeptide.
Embodiment 56. The vesicle of embodiment 54, wherein said second inhibitor inhibits expression or activity of said TAZ polypeptide.
Embodiment 57. The vesicle of embodiment 54, wherein said second inhibitor inhibits expression or activity of said LCK polypeptide.
Embodiment 58. The vesicle of any one of embodiments 50-52, wherein said vesicle comprises a second inhibitor located within said vesicle and a third inhibitor located within said vesicle.
Embodiment 59. The vesicle of embodiment 58, wherein said second inhibitor and said third inhibitor inhibits expression or activity of a YAP polypeptide, a TAZ polypeptide, or said LCK polypeptide.
Embodiment 60. The vesicle of any one of embodiments 50-52, wherein said vesicle comprises a second inhibitor located within said vesicle, a third inhibitor located within said vesicle, and a fourth inhibitor located within said vesicle.
Embodiment 61. The vesicle of embodiment 60, wherein said second inhibitor inhibits expression or activity of a YAP polypeptide, wherein said third inhibitor inhibits expression or activity of a TAZ polypeptide, and wherein said fourth inhibitor inhibits expression or activity of said LCK polypeptide.
Embodiment 62. The vesicle of any one of embodiments 36-61, wherein said vesicle comprises an imaging agent.
Embodiment 63. The vesicle of embodiment 62, wherein said imaging agent is selected from the group consisting of a fluorophore, a fluorescent dye, and a radionucleotide.
Embodiment 64. The vesicle of any one of embodiments 36-63, wherein said cancer cell is located within a liver, gall bladder, small intestine, or bile duct of a mammal.
Embodiment 65. The vesicle of embodiment 64, wherein said mammal is a human.
Embodiment 66. The vesicle of any one of embodiments 36-65, wherein said cancer cell is a liver cancer cell, a gall bladder cancer cell, a small intestine cancer cell, or a bile duct cancer cell.
Embodiment 67. The vesicle of any one of embodiments 36-65, wherein said cancer cell is a cholangiocarcinoma cancer cell.
Embodiment 68. The vesicle of any one of embodiments 36-67, wherein said molecule is an EpCAM polypeptide, an EPHA2 polypeptide, an AXL polypeptide, a GPRC5a polypeptide, a GPRC5c polypeptide, an EGFR polypeptide, a CK19 polypeptide, an SPP1 polypeptide, or a Sox9 polypeptide.
Embodiment 69. The vesicle of any one of embodiments 36-68, wherein said targeting moiety is an antibody.
Embodiment 70. The vesicle of any one of embodiments 36-68, wherein said targeting moiety is a nucleic acid aptamer.
Embodiment 71. The vesicle of embodiment 70, wherein said nucleic acid aptamer is an EpCAM aptamer, an AXL aptamer, an EGFR aptamer, a CK19 aptamer, a SPP1 aptamer, or a Sox9 aptamer.
Embodiment 72. The vesicle of any one of embodiments 70-71, wherein said nucleic acid aptamer is an RNA aptamer.
Embodiment 73. The vesicle of any one of embodiments 70-71, wherein said nucleic acid aptamer is a DNA aptamer.
Embodiment 74. The vesicle of any one of embodiments 36-73, wherein said longest diameter is 100 nm to 500 nm.
Embodiment 75. The vesicle of any one of embodiments 36-74, wherein said longest diameter is 200 nm to 400 nm.
Embodiment 76. A method for treating cancer, wherein said method comprises administering a composition comprising a vesicle of any one of claims 1-75 to a mammal having cancer, wherein the number of cancer cells within said mammal is reduced following said administering of said composition.
Embodiment 77. The method of embodiment 76, wherein said mammal is a human.
Embodiment 78. The method of any one of embodiments 76-77, wherein said cancer cells are liver cancer cells, gall bladder cancer cells, small intestine cancer cells, or bile duct cancer cells.
Embodiment 79. The method of any one of embodiments 76-78, wherein said cancer is cholangiocarcinoma.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A vesicle comprising a first inhibitor located within said vesicle, a second inhibitor located within said vesicle, a third inhibitor located within said vesicle, and a targeting moiety located on a surface of said vesicle, wherein said first inhibitor inhibits expression or activity of a first polypeptide, wherein said second inhibitor inhibits expression or activity of a second polypeptide, wherein said third inhibitor inhibits expression or activity of a third polypeptide, wherein said first polypeptide, said second polypeptide, and said third polypeptide are selected from the group consisting of a YAP polypeptide, a TAZ polypeptide, a LCK polypeptide, and a TRAIL polypeptide, wherein said targeting moiety binds to a molecule present on the surface of a cancer cell, and wherein said vesicle has a longest diameter of 5 nm to 500 nm.

2-11. (canceled)

12. The vesicle of claim 1, wherein said vesicle comprises a fourth inhibitor located within said vesicle, wherein said fourth inhibitor inhibits expression or activity of a fourth polypeptide.

13. (canceled)

14. The vesicle of claim 1, wherein said vesicle comprises an imaging agent.

15. The vesicle of claim 14, wherein said imaging agent is selected from the group consisting of a fluorophore, a fluorescent dye, and a radionucleotide.

16. The vesicle of claim 1, wherein said cancer cell is located within a liver, gall bladder, small intestine, or bile duct of a mammal.

17. The vesicle of claim 16, wherein said mammal is a human.

18. The vesicle of claim 1, wherein said cancer cell is a liver cancer cell, a gall bladder cancer cell, a small intestine cancer cell, or a bile duct cancer cell.

19. The vesicle of claim 1, wherein said cancer cell is a cholangiocarcinoma cancer cell.

20-25. (canceled)

26. A vesicle comprising an inhibitor located within said vesicle and a targeting moiety located on a surface of said vesicle, wherein said inhibitor inhibits expression or activity of a LCK polypeptide or a TRAIL polypeptide, wherein said targeting moiety binds to a molecule present on the surface of a cancer cell, and wherein said vesicle has a longest diameter of 5 nm to 500 nm.

27. The vesicle of claim 26, wherein said vesicle comprises an imaging agent.

28. The vesicle of claim 27, wherein said imaging agent is selected from the group consisting of a fluorophore, a fluorescent dye, and a radionucleotide.

29. The vesicle of claim 26, wherein said cancer cell is located within a liver, gall bladder, small intestine, or bile duct of a mammal.

30. The vesicle of claim 29, wherein said mammal is a human.

31. The vesicle of claim 26, wherein said cancer cell is a liver cancer cell, a gall bladder cancer cell, a small intestine cancer cell, or a bile duct cancer cell.

32. The vesicle of claim 26, wherein said cancer cell is a cholangiocarcinoma cancer cell.

33-38. (canceled)

39. A method for treating cancer, wherein said method comprises administering a composition comprising a vesicle to a mammal having cancer, wherein the number of cancer cells within said mammal is reduced following said administering of said composition, wherein said vesicle comprises a first inhibitor located within said vesicle, a second inhibitor located within said vesicle, a third inhibitor located within said vesicle, and a targeting moiety located on a surface of said vesicle, wherein said first inhibitor inhibits expression or activity of a first polypeptide, wherein said second inhibitor inhibits expression or activity of a second polypeptide, wherein said third inhibitor inhibits expression or activity of a third polypeptide, wherein said first polypeptide, said second polypeptide, and said third polypeptide are selected from the group consisting of a YAP polypeptide, a TAZ polypeptide, a LCK polypeptide, and a TRAIL polypeptide, wherein said targeting moiety binds to a molecule present on the surface of a cancer cell, and wherein said vesicle has a longest diameter of 5 nm to 500 nm.

40. The method of claim 39, wherein said mammal is a human.

41. The method of claim 39, wherein said cancer cells are liver cancer cells, gall bladder cancer cells, small intestine cancer cells, or bile duct cancer cells.

42. The method of claim 39, wherein said cancer is cholangiocarcinoma.

43-46. (canceled)
